(19) United States
(12) Patent Application Publication
(10) Pub. No.: US 2024/0425937 A1
(43) Pub. Date: Dec. 26, 2024

(54) METHODS OF DIAGNOSIS AND TREATMENT OF EPILEPSY BASED ON GASTROINTESTINAL MICROBIOTA ALTERATIONS

(71) Applicant: NEW YORK UNIVERSITY, New York, NY (US)

(72) Inventors: Deepak SAXENA, New York, NY (US); Claude STERIADE, Garden City, NY (US)

(73) Assignee: NEW YORK UNIVERSITY, New York, NY (US)

(21) Appl. No.: 18/749,550

(22) Filed: Jun. 20, 2024

Related U.S. Application Data
(60) Provisional application No. 63/522,055, filed on Jun. 20, 2023.

(51) Int. Cl. C12Q 1/6888; C12Q 1/6874; G01N 33/68

(52) U.S. Cl. CPC C12Q 1/6888; C12Q 1/6874; G01N 33/6863; C12Q 2600/112

(57) ABSTRACT

The application relates to methods of diagnosis of status epilepticus (SE) or new onset refractory status epilepticus (NORSE), as well as determining the likelihood of mortality associated with SE and/or NORSE, based on mammalian gastrointestinal (GI) microbiota. The application further relates to the treatment or prevention of SE and/or NORSE, or mortality associated with these conditions via use of probiotics, prebiotics, compounds and/or compositions for modulating the GI microbiota.

### CROSS-REFERENCE TO RELATED APPLICATIONS

This patent application claims priority to U.S. Provisional Patent Application No. 63/522,055, filed on Jun. 20, 2023, which is incorporated herein by reference in its entirety.

### FIELD

The application relates to methods of diagnosis of status epilepticus (SE) or new onset refractory status epilepticus (NORSE), as well as determining the likelihood of mortality associated with SE and/or NORSE, based on mammalian gastrointestinal (GI) microbiota. The application further relates to the treatment or prevention of SE and/or NORSE, or mortality associated with these conditions via use of probiotics, prebiotics, compounds and/or compositions for modulating the GI microbiota.

### BACKGROUND

Status epilepticus (SE), defined as a prolonged seizure or multiple seizures without return to baseline, is a neurologic emergency with high morbidity and mortality. (Betjemann & Lowenstein, 2015) Within SE, a subset of patients may continue to seize despite appropriate treatment, increasing the risk of neurologic sequelae. (Rossetti & Lowenstein, 2011) New onset refractory status epilepticus (NORSE) encompasses refractory SE without pre-existing epilepsy or an active structural, toxic, or metabolic cause of seizures. (Hirsch et al., 2018) NORSE is rare, with an estimated incidence rate of 2 cases per 100,000 per year, (Ritter & Nashef, 2021; Sculier & Gaspard, 2019) with limited evidence for clinical management recommendations. (Hanin, Cespedes, Huttner, et al., 2023; Sheikh & Hirsch, 2023) Despite its low incidence, the morbidity and mortality of NORSE is high. One fifth die during acute illness. (Tharmaraja et al., 2023) Among those who survive, one half suffer disabling symptoms, over half continue to experience seizures despite medication, (Gaspard et al., 2015; Taraschenko et al., 2023) and many experience lasting significant neuropsychological sequelae. (Shrestha et al., 2023; Taraschenko et al., 2023) The high morbidity and mortality translates into high cost. (Strzelczyk et al., 2017)

Inflammation has been posited as a potential mechanism of neurologic injury in SE (PMID: 31171434). Pharmacologic blockade of various inflammatory pathways (e.g. L-1β/IL-1R1 axis, HMGB1, P2X7 receptors) can reduce the duration and severity of SE in experimental models of SE (PMID: 36397618). Upregulation of many proinflammatory cytokines (including IL-6, IL-1β, TNF-α, CXCL8/IL-8, CCL2, MIP-1α, and IL-12p70) has been specifically documented in NORSE, with some cytokines correlating with both short- and long-term outcomes. (Hanin, Cespedes, Dorgham, et al., 2023; Kwack & Kim, 2022; Sakuma et al., 2015) The mechanism for this cytokine upregulation is unknown.

Gut dysbiosis is a promising mechanism underpinning inflammation in SE and NORSE. Gut microbiota are known to have a broad impact on the immune system through activation of pattern-recognition receptors and regulation of lymphocyte subsets in the gut. (Kamada et al., 2013) Gut dysbiosis modulates cytokine responses in healthy subjects (Schirmer et al., 2016) and those with brain injury. (Singh et al., 2016) Specifically, gut microbiota induces IL-1β production, which mediates Thl7 cell differentiation in a species-specific manner. (Kamada et al., 2013; Shaw et al., 2012) Preliminary investigations of gut microbiota in a number of neurologic autoimmune conditions, neurodegenerative conditions (Tremlett et al., 2017), and epilepsy (PMID 33140419) have supported a potential role of gut dysbiosis in mediating neuroinflammation and neurologic dysfunction.

Individuals affected by SE and NORSE are most often treated for weeks in an intensive care unit because they require prolonged anesthesia with coma-inducing drugs to control their seizures. SE and NORSE also carry a high rate of complications and mortality, epilepsy and/or cognitive issues. There are financial costs that are associated with this increased morbidity, disability, and premature death. These affect individuals, families, communities, and our society in general. Thus, there remains a need for methods for earlier and more accurate diagnosing, treating, and preventing conditions such as SE and NORSE or determining the predisposition to worse outcomes such as mortality, disability, epilepsy, cognitive impairment, neuroinflammation, and/or neurodegeneration, such as methods and treatments based on or related to the gut microbiome, including associated antigens.

### SUMMARY OF THE INVENTION

As specified in the Background section above, there is a great need for methods for earlier and more accurate diagnosing, treating, and preventing conditions such as SE and NORSE or determining the predisposition to worse outcomes.

In one aspect, the invention provides a method for the diagnosis of a subject suffering from Status Epilepticus (SE) or at risk of suffering from SE, said method comprising: (a) determining in a fecal microbiota sample isolated from the subject the level of at least one strain of fungi from one or more class Saccharomycetales or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Enterococcus faecalis, Enterocloster bolteae, Escherichia coli, Sellimonas intestinalis, Akkermansia muciniphila, Fusicatenibacter saccharivorans, Lacticaseibacillus rhamnosus, Streptococcus anginosus, and Roseburia hominis or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject has SE or at risk of suffering from SE, wherein the level of at least one of the strains determined in step (a) is higher than in the control. In certain aspects, the strain is derived from the genera of the species listed herein.

In one aspect, the invention provides a method for the diagnosis of a subject suffering from Status Epilepticus (SE) or at risk of suffering from SE, said method comprising: (a) determining in a fecal microbiota sample isolated from the subject the level of at least one strain of fungi from one or more species selected from Trichoderma breve and Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Clostridium leptum, Bacteroides stercoris, Fusicatenibacter saccharivorans, and Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject has SE or at risk of suffering from SE, wherein the level of at least one of the strains determined in step (a) is lower than in the control. In certain aspects, the strain is derived from the genera of the species listed herein.

In one aspect, the invention provides a method for the diagnosis of a subject suffering from New Onset Refractory Status Epilepticus (NORSE) or at risk of suffering from NORSE, said method comprising: (a) determining in a fecal microbiota sample isolated from the subject the level of at least one strain of fungi from genus Nakaseomyces glabratus and Marasmius oreades or closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Enterococcus faecalis, Enterococcus faecium, Escherichia coli, Enterocloster bolteae, Lacticaseibacillus paracasei, and an Clostridiaceae_bacterium_OM02_2AC species closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject is predisposed to NORSE or at risk of suffering from NORSE, wherein the level of at least one of the strains determined in step (a) is higher than in the control. In certain aspects, the strain is derived from the genera of the species listed herein.

In one aspect, the invention provides a method for the diagnosis of a subject suffering from New Onset Refractory Status Epilepticus (NORSE) or at risk of suffering from NORSE, said method comprising: (a) determining in a fecal microbiota sample isolated from the subject the level of at least one strain of fungi from one or more species selected from Trichoderma breve and Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species from Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject has NORSE or at risk of suffering from NORSE, wherein the level of at least one of the strains determined in step (a) is lower than in the control. In certain aspects, the strain is derived from the genera of the species listed herein.

In certain embodiments, the level of at least one of the strains determined in step (a) is at least 2-log-fold to 10-log-fold higher than in the control. In certain embodiments, the level of at least one of the strains determined in step (a) is at least 2-log-fold to 10-log-fold lower than in the control. In certain embodiments, the control fecal microbiota is fecal microbiota of chronic epilepsy subjects.

In one aspect, the invention provides a method for predicting risk of mortality associated with SE or NORSE a subject presenting with SE or NORSE, said method comprising: (a) determining in a fecal microbiota sample isolated from the subject the level of at least one strain of fungi from one or more species selected from Family Saccharomycetaceae or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Enterococcaceae, Genus Enterococcus, and Species Enterococcus faecalis, or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in the control fecal microbiota, and (c) identifying that the subject is at risk of mortality associated with SE or NORSE, wherein the level of at least one of the strains determined in step (a) is higher than in the control. In certain aspects, the strain is derived from the genera of the species listed herein. In certain embodiments, the level of at least one of the strains determined in step (a) is at least 2-log-fold to 10-log-fold higher than in the control.

In one aspect, the invention provides a method for predicting risk of mortality associated with SE or NORSE in a subject presenting with SE or NORSE, said method comprising: (a) determining in a fecal microbiota sample isolated from the subject the level of at least one strain of fungi from one or more species selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castellii, Species Ogataea parapolymorpha, Species Pyricularia pennisetigena, Species Saccharomyces kudriavzevii, Species Saccharomyces mikatae, and Species Yarrowia lipolytica or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Atopobiaceae, Family Bifidobacteriaceae, Family Clostridiaceae, Family FGB2982 c CFGB2982 p Firmicutes, Family Lachnospiraceae, Family Oscillospiraceae, Family Staphylococcaceae, Genus Bifidobacterium, Genus Blautia, Genus Coprococcus, Genus Dorea, Genus GGB9342 f FGB2982 c CFGB2982 p Firmicutes, Genus GGB9699 f Oscillospiraceae, Genus Staphylococcus, Genus Streptococcus, Species Bifidobacterium dentium, Species Blautia obeum, Species Dorea longicatena, Species SGB14306 g GGB9342 f FGB2982 c CFGB2982 p Firmicutes, and Species SGB15216 g GGB9699f Oscillospiraceae or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject is at risk of mortality associated with SE or NORSE, wherein the level of at least one of the strains determined in step (a) is lower than in the control. In certain aspects, the strain is derived from the genera of the species listed herein. In certain embodiments, the level of at least one of the strains determined in step (a) is at least 2-log-fold to 10-log-fold lower than in the control.

In certain embodiments, the control fecal microbiota is fecal microbiota of subjects presenting with SE or NORSE that did not experience mortality. In certain embodiments, testing occurs 1, 2, 3, 4, 5, 6, 7, 8, 9, or 10 years after experiencing SE or NORSE.

In certain embodiments, the level of fungi and/or bacteria is determined by a method selected from shotgun metagenomics, quantitative PCR (qPCR), high-throughput sequencing, transcriptomic analysis, bacterial or fungal genotype pattern based fingerprinting (DNA fingerprinting), inflammatory cytokine, metabolomics, bacterial or fungal gene profiling, and proteomic analysis.

In certain embodiments, the fecal sample had been isolated from the subject during status epilepticus or within 3 days of status epilepticus resolution.

In certain embodiments, the method further comprises isolating the fecal sample from the subject prior to step (a).

In certain embodiments, determining the level of at least one strain of fungi and/or bacteria comprises extracting DNA from bacterial and fungal species at the same time. In certain embodiments, the method further comprises determining the level of one or more cytokines in a blood sample from the subject. In certain embodiments, the subject has SE or is likely to develop SE when the level of at least one of GCSF, IL10, IL12p70, IL1b, IL4, TNFa, and IL17A in the sample is higher than in a control sample. In certain embodiments, the subject has NORSE or is likely to develop NORSE when the level of at least one of IL6, CCL2, GCSF, and IL1b in the sample is higher than in the control sample.

In certain embodiments, the method further comprises recruiting the subject in a clinical trial.

In certain embodiments, the method further comprises administering one or more treatments to the subject. In certain embodiments the treatment comprises administering an effective amount of a compound inhibiting upregulation of IL-10, IL-6, and IL-10. In certain embodiments the treatment comprises administering an effective amount of anakinra, tocilizumab, or biologics targeting cytokine upregulation in SE and NORSE to the subject. In certain embodiments the treatment comprises administering an effective amount of a compound, composition, probiotic, and/or a prebiotic that stimulates growth and/or activity of one or more strains of fungi and/or bacteria which level determined in step (a) is lower than in the control. In certain embodiments the treatment comprises administering an effective amount of a probiotic comprising one or more strains of fungi and/or bacteria, diet modification, or fecal microbiota transplantation (FMT), which level determined in step (a) is lower than in the control. In certain embodiments the route of administration for said probiotic or FMT comprises at least one of upper gastrointestinal routes (UGI) (such as nasogastric/nasojejunal tube, endoscopy, or oral capsules) or lower gastrointestinal routes (LGI) (such as retention enema, sigmoidoscopy or colonoscopy).

In one aspect, the invention provides a method for treating or preventing Status Epilepticus (SE) in a subject in need thereof, said method comprising: (a) detecting in the fecal microbiota sample isolated from the subject the level of at least one strain of SGB14043_p_Firmicutes, and (b) administering an effective amount of a compound inhibiting upregulation of IL-6, IL-8, and/or CCL2 in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Akanthomyces and Species Akanthomyces muscarius, and (b) administering an effective amount of a compound inhibiting upregulation of IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Bifidobacterium and Species Bifidobacterium longum, and (b) administering an effective amount of a compound inhibiting upregulation of IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Blautia and Species Blautia wexlerae, and (b) administering an effective amount of a compound inhibiting upregulation of CCL2 in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Colletotrichum and Species Colletotrichum higginsianum, and (b) administering an effective amount of a compound inhibiting upregulation of IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Collinsella and Species Collinsella aerofaciens, and (b) administering an effective amount of a compound inhibiting upregulation of IL-1b and/or IL12p70 in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Enterobacter and Species Enterobacter hormaechei, and (b) administering an effective amount of a compound inhibiting upregulation of MIP1a, GCSF, IL12p70, IL-4, TNFa and/or IL-17a in the subject.

In one aspect, the invention provides a A method for treating or preventing Status Epilepticus (SE) in a subject in need thereof, said method comprising: (a) detecting in the fecal microbiota sample isolated from the subject the level of at least one strain selected from Genus Enterococcus and Species Enterococcus faecalis, and (b) administering an effective amount of a compound inhibiting upregulation of VEGF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Lacticaseibacillus and Species Lacticaseibacillus rhamnosus, and (b) administering an effective amount of a compound inhibiting upregulation of VEGF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Megamonas and Species Megamonas funiformis, and (b) administering an effective amount of a compound inhibiting upregulation of GCSF and/or IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Naumovozyma and Species Naumovozyma dairenensis, and (b) administering an effective amount of a compound inhibiting upregulation of GCSF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Penicillium and Species Penicillium oxalicum, and (b) administering an effective amount of a compound inhibiting upregulation of CCL2 in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Pyricularia and Species Pyricularia pennisetigena, and (b) administering an effective amount of a compound inhibiting upregulation of IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Ruminococcus and Species Ruminococcus torques, and (b) administering an effective amount of a compound inhibiting upregulation of GCSF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Talaromyces and Species Talaromyces rugulosus, and (b) administering an effective amount of a compound inhibiting upregulation of GCSF and/or IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Tetrapisispora and Species Tetrapisispora blattae, and (b) administering an effective amount of a compound inhibiting upregulation of GCSF, IL12p70, and/or IL-1b in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Candida and Species Candida dubliniensis, and (b) administering an effective amount of a compound inhibiting upregulation of IL12p70, IL-4, TNFa and/or IL-17a in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Bacteroides and Species Bacteroides stercoris, and (b) administering an effective amount of a compound stimulating upregulation of CCL2 in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Candida and Species Candida dubliniensis, and (b) administering an effective amount of a compound stimulating upregulation of MIP1a in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Enterococcus and Species Enterococcus faecium, and (b) administering an effective amount of a compound stimulating upregulation of GCSF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Genus Nakaseomyces and Species Nakaseomyces glabratus, and (b) administering an effective amount of a compound stimulating upregulation of GCSF in the subject.

In one aspect, the invention provides a method for treating or preventing Status Epilepticus (SE) in a subject in need thereof, said method comprising: (a) detecting in the fecal microbiota sample isolated from the subject the level of at least one strain selected from Phylum Firmicutes and SGB14043_p_Firmicutes, and (b) administering an effective amount of a compound stimulating upregulation of MIP1a and/or IL-10 in the subject.

In one aspect, the invention provides a A method for treating or preventing Status Epilepticus (SE) in a subject in need thereof, said method comprising: (a) detecting in the fecal microbiota sample isolated from the subject the level of at least one strain selected from Genus Torulaspora and Species Torulaspora delbrueckii, and (b) administering an effective amount of a compound stimulating upregulation of CCL2 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Akkermansia and Species Akkermansia muciniphila, and (b) administering an effective amount of a compound inhibiting upregulation of CCL2 and/or GCSF in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Alistipes and Species Alistipes onderdonkii, and (b) administering an effective amount of a compound inhibiting upregulation of IL-4 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Candida and Species Candida dubliniensis, and (b) administering an effective amount of a compound inhibiting upregulation of CCL2, IL12p70, and/or TNFa in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Enterocloster and Species Enterocloster bolteae, and (b) administering an effective amount of a compound inhibiting upregulation of IL-6, CCL2, and/or IL-1b in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Enterococcus and Species Enterococcus faecalis, and (b) administering an effective amount of a compound inhibiting upregulation of VEGF, IL12p70, and/or TNFa in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Eremothecium and Species Eremothecium gossypii, and (b) administering an effective amount of a compound inhibiting upregulation of IL-4 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Escherichia and Species Escherichia coli, and (b) administering an effective amount of a compound inhibiting upregulation of MIP1a, IL-10, and/or IL-17a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Fusarium and Species Fusarium venenatum, and (b) administering an effective amount of a compound inhibiting upregulation of IL-10 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Malassezia and Species Malassezia restricta, and (b) administering an effective amount of a compound inhibiting upregulation of IL-10 and/or IL-17a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Nakaseomyces and Species Nakaseomyces glabratus, and (b) administering an effective amount of a compound inhibiting upregulation of IL-10 and/or IL-17a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Psilocybe and Species Psilocybe cubensis, and (b) administering an effective amount of a compound inhibiting upregulation of IL-10 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Purpureocillium and Species Purpureocillium takamizusanense, and (b) administering an effective amount of a compound inhibiting upregulation of IL-10 in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Akanthomyces and Species Akanthomyces muscarius, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Akkermansia and Species Akkermansia muciniphila, and (b) administering an effective amount of a compound stimulating upregulation of IL-6 and/or IL-1b in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Bifidobacterium and Species Bifidobacterium longum, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Phylum Candidatus, Genus Candidatus Cibionibacter, and Species Candidatus Cibionibacter quicibialis, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Drechmeria and Species Drechmeria coniospora, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Enterocloster and Species Enterocloster bolteae, and (b) administering an effective amount of a compound stimulating upregulation of GCSF in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Malassezia and Species Malassezia restricta, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Nakaseomyces and Species Nakaseomyces glabratus, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Psilocybe and Species Psilocybe cubensis, and (b) administering an effective amount of a compound stimulating upregulation of MIP1a and/or IL-17a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Purpureocillium and Species Purpureocillium takamizusanense, and (b) administering an effective amount of a compound stimulating upregulation of IL-17a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Ruminococcus and Species Ruminococcus bromii, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Ruminococcus and Species Ruminococcus torques, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Schizosaccharomyces and Species Schizosaccharomyces osmophilus, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Thermothielavioides and Species Thermothielavioides terrestris, and (b) administering an effective amount of a compound stimulating upregulation of MIP1a in the subject.

In one aspect, the invention provides a method for treating or preventing New Onset Refractory Status Epilepticus (NORSE) in a subject in need thereof, said method comprising: (a) detecting in the fecal microbiota sample isolated from the subject the level of at least one strain selected from Genus Trichoderma and Species Trichoderma breve, and (b) administering an effective amount of a compound stimulating upregulation of MIP1a in the subject. In certain embodiments, the subject is human.

These and other aspects of the present invention will be apparent to those of ordinary skill in the art in the following description, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J. The microbiomes of NORSE and SE cohorts are different than the chronic epilepsy control cohort (NORSE and SE T1 used). Species-level taxonomic alpha diversity for Prokaryotes (1A) and Eukaryotes (1B), and functional alpha diversity at the gene family level (UniRef90) (1C) and pathway level (1D) (Wilcoxon rank-sum test; *, p<0.05; **, p<0.01). Microbiome beta diversity between all cohorts (1E-1H) and pair-wise comparisons between the epilepsy control cohort and NORSE or SE (1I-1J) (PCoA of Bray-Curtis dissimilarity, PERMANOVA). Shannon Diversity Index (sometimes called the Shannon-Wiener Index) is a way to measure the diversity of species in a community by assuming all species are represented in a sample and that they are randomly sampled.

FIG. 2A-2C. Microbiome features are significantly enriched or depleted with respect to chronic epilepsy control cohort. Prokaryotic (2A) or Eukaryotic (2B) species, and pathways (MetaCyc nomenclature) (2C) that are significantly enriched or depleted in SE or NORSE at T1 with respect to chronic epilepsy control cohort. Significance for species was determined with LEfSe.

FIG. 3A-3L. Diversity differences between and within SE and NORSE cohorts. Prokaryotic (3A), Eukaryotic (3B), gene family (3C), and pathway (3D) alpha diversity between NORSE and SE (3A-3D). Beta diversity between (3E-3H, 3K, and 3L), and within cohorts (3I and 3J).

FIG. 4A-4C. Microbiome features that are significantly enriched or depleted across or within SE and NORSE cohorts for Prokaryotes (4A), Eukaryotes (4B), or Pathways (4C) are noted. Top 40 species mean relative abundance heat maps (row z-score scaled) with hierarchical clustering based on species abundance patterns across cohorts and time points (4A and 4B).

FIG. 5A-5D. Plasma cytokine abundance plots for SE and NORSE showing across and within cohort comparisons (Wilcoxon rank sum test). The abundance plots show plasma cytokine levels at T1 (5A), plasma cytokine levels at T2 (5B), plasma cytokine levels for SE at T1 and T2 (5C), and plasma cytokine levels for NORSE at T1 and T2 (5D).

FIG. 6A-6B. Microbial species and plasma cytokine correlation plots for SE (6A) and NORSE (6B). Spearman's rank correlation coefficients between the relative abundance of the top 20 prokaryotic and eukaryotic species, and plasma inflammatory cytokine levels (p<0.05, *; p<0.01, **; p<0.001, ***). The metagenomic sample closest in time to the plasma sample was used, in the case of a tie before and after plasma sampling, the later time point metagenomic sample was chosen.

FIG. 7A-7F. Prokaryotic alpha and beta diversity for mortality (7A, 7C) and tube feeding (7B, 7D) (latest T2 sample used). Between mortality outcomes, surviving patients harbored significantly higher prokaryotic species alpha diversity than patients who passed away (7A). Between feeding outcomes, orally fed patients harbored significantly higher prokaryotic Shannon diversity than patients who passed away (7B). Prokaryotic beta diversity analysis (species-level) indicates different microbial communities were associated with patients who passed away during the observation period and those who survived, and a greater degree of variance in community composition was noted for patients who passed away during the observation period (7C). Prokaryotic beta diversity analysis (species-level) indicated different microbial communities were associated with patients who were tube fed than orally fed, and a greater degree of variance in community composition was noted for patients who were tube fed (7D). Fisher's exact test (7E-7F) shows the outcomes of feeding or mortality is not significantly associated with presenting with NORSE or SE.

FIG. 8A-8B. Antibiotic action beta diversity. If all samples associated with an antibiotic action, significant differences are found (8A). If a single sample per patient (earliest, 8B PCoA) is used no significant differences in taxonomic community composition is found. Groupings are (determined based on sample size available and there are many patients with multiple antibiotic use) 1=Cell wall, 2=cell wall and nucleic acid synthesis; 3=cell wall and protein synthesis; 4=cell wall and protein synthesis and nucleic acid synthesis; 5=others (cell wall and DNA/oxidative damage; cell wall and cell membrane and protein synthesis and nucleic acid synthesis; cell wall and cell membrane and protein synthesis).

FIG. 9A-9B. Microbiome features that are significantly enriched or depleted across or within SE and NORSE, oral and tube feeding, and mortality and non-mortality cohorts for Eukaryotes (Fungi). Top 20 genus (9A) and top 40 species (9B) are noted.

FIG. 10A-10B. Microbiome features that are significantly enriched or depleted across or within SE and NORSE, oral and tube feeding, and mortality and non-mortality cohorts for Prokaryotes. Top 20 genus (10A) and top 40 species (10B) are noted.

### DETAILED DESCRIPTION

The present disclosure provides methods and compositions for diagnosing and treating (including preventing) Status Epilepticus (SE) or New Onset Refractory Status Epilepticus (NORSE) and determining the likelihood of mortality associated with SE or NORSE in subjects presenting with SE or NORSE. In some embodiments, the methods comprise determining the likelihood of a worse outcome associated with SE or NORSE in subjects presenting with SE or NORSE. In certain embodiments, the worse outcome can be mortality, disability, epilepsy, cognitive impairment, neuroinflammation, and/or neurodegeneration. In certain embodiments, the worse outcome can be mortality.

The present disclosure is based on an unexpected discovery that subjects diagnosed with SE and/or NORSE demonstrated significant differences in microbiome structure compared to subjects with chronic epilepsy. Over the course of SE resolution and hospitalization, microbiome diversity changed differently in those with SE of known cause and those with NORSE. Specific microbiota taxa correlated with cytokines known to be upregulated in NORSE and SE, providing insight into inflammation as one possible mechanism through which microbiome changes may impact neurologic injury in SE. Oral feeding and specific microbiome profiles predict survival in SE, further supporting a role for the gut microbiome in impacting disease course and neurologic injury in SE. Data in support of each of these findings is presented in the Examples section, below.

The invention disclosed herein can enable methods for treating a subject diagnosed with SE or NORSE. In some embodiments, the methods comprise treating SE or NORSE, in a subject diagnosed with SE or NORSE.

The invention can also enable methods of monitoring the effect of a treatment on development of a disorder in a subject, wherein the disorder is SE or NORSE. In some embodiments, the subject is diagnosed with SE or NORSE.

### Definitions

As used herein, the terms “microbe”, “microorganism”, “microbial”, or “microbiota” encompass both prokaryotic organisms including bacteria and archaea, and eukaryotic organisms, including fungi, present in mammalian microbiota, and viruses.

The terms “gastrointestinal microbiota” and “GI microbiota” are used interchangeably and refer to the microorganisms that colonize the gastrointestinal system. The gastrointestinal system includes the mouth, pharynx (throat), esophagus, stomach, small intestine, large intestine, rectum, and anus. In certain embodiments, the gastrointestinal system also includes the salivary glands, liver, gallbladder, and pancreas. In certain embodiments, “GI microbiota” refers specifically to “intestinal microbiota”, “intestinal flora”, and the “intestinal microbiome”.

As used herein, the term “dysbiosis” refers to a microbial imbalance on or inside the body. Dysbiosis can result from, e.g., antibiotic or anti-fungal exposure as well as other causes, e.g., infections with pathogens including viruses, bacteria and eukaryotic parasites.

The term “alpha diversity” as used herein refers to the diversity of species in a site within a localized area (e.g., such as within the gastrointestinal tract).

The term “beta diversity” as used herein refers to a measure of comparing samples from different groups to determine overall community composition and structural differences.

Specific taxa and changes in GI microbiota discussed herein can be detected using various methods, including without limitation quantitative PCR or high-throughput sequencing methods which detect over- and under-represented genes in the total microbial population (e.g., 454-sequencing for community analysis; screening of microbial 16S ribosomal RNAs (16S rRNA), or 28S rRNA, 18S internal transcribed space (ITS) sequencing, etc.), or transcriptomic or proteomic studies that identify lost or gained microbial transcripts or proteins within total bacterial populations. See, e.g., U.S. Patent Publication No. 2010/0074872; Eckburg et al., Science, 2005, 308:1635-8; Costello et al., Science, 2009, 326:1694-7; Grice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703, each of which are herein incorporated by reference in their entirety for all intended purposes.

As used herein, the term “16S rRNA sequencing” refers to the sequencing of 16S ribosomal RNA (rRNA) gene sequences by using primers such as universal primers and/or species-specific primers to identify the bacteria present in a sample. 16S rRNA genes contain both highly conserved sites and hypervariable regions that can provide species-specific signature sequences useful for identification of bacteria. Such universal primers are well known in the art.

As used herein, the term “18S rRNA sequencing” refers to the sequencing of 18S ribosomal RNA (rRNA) gene sequences by using primers such as universal primers and/or species-specific primers to identify the fungi present in a sample. 18S rRNA genes contain both highly conserved sites and hypervariable regions that can provide species-specific signature sequences useful for identification of fungi. Such universal primers are well known in the art.

As used herein, the term “operational taxonomic unit” or “OTU” refers to group of bacterial sequences that differ among each other in <97% identity. A “type” or a plurality of “types” of bacteria includes an OTU or a plurality of different OTUs, and also encompasses differences in species, genus, family or order of fungus or bacteria. The specific genetic sequence may be the 16S rRNA sequence or a portion of the 16S rRNA sequence or it may be a functionally conserved housekeeping gene found broadly across the eubacterial kingdom. The specific genetic sequence may be the 18S rRNA sequence or a portion of the 18S rRNA sequence or it may be a functionally conserved housekeeping gene found broadly across the fungal kingdom.

As used herein, the term “abundance” refers to how common or rare a particular organism (e.g., fungal or bacterial species) is relative to other organisms of the same type (e.g., other fungal or bacterial species) in a defined community. In certain embodiments, abundance is the percent composition of a particular organism (e.g., fungal or bacterial species) to the total amount of organisms in the sample. In certain embodiments, abundance refers to the total level of organism in a sample. In certain embodiments, abundances are described for a single trophic level.

As used herein, the term “probiotic” refers to a substantially pure bacteria (i.e., a single isolate, of, e.g., live bacterial cells, conditionally lethal bacterial cells, inactivated bacterial cells, killed bacterial cells, spores, recombinant carrier strains), pure fungi (i.e., a single isolate, of, e.g., live fungal cells, conditionally lethal fungal cells, inactivated fungal cells, killed fungal cells, spores, recombinant carrier strains), or a mixture of desired bacteria and/or fungi, bacteria and/or fungi components or bacterial and/or fungi extract, or bacterially- or fungally-derived products (natural or synthetic products such as, e.g., bacterial and/or fungal antigens or metabolic products) and may also include any additional components that can be administered to a mammal. Such compositions are also referred to herein as “bacterial inoculants”, “fungal inoculants”, or “microbiota inoculants”. Probiotics, fungal inoculant compositions, or bacterial inoculant compositions of the invention may be administered with a buffering agent to allow the bacteria and/or fungi to survive in the acidic environment of the stomach, i.e., to resist low pH and to grow in the intestinal environment. Such buffering agents include sodium bicarbonate, juice, milk, yogurt, infant formula, and other dairy products.

As used herein, the term “prebiotic” refers to an agent that simulates the growth (e.g., increases the number) and/or activity of one or more desired bacteria and/or fungi. Non-limiting examples of prebiotics useful in the methods of the present invention include fructooligosaccharides (e.g., oligofructose, inulin, inulin-type fructans), galactooligosaccharides, human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, other five- and six-carbon sugars (such as arabinose, maltose, lactose, sucrose, cellobiose, etc.), amino acids, alcohols, resistant starch (RS), water-soluble cellulose derivatives (most preferably, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose), water-insoluble cellulose derivatives (most preferably, ethyl cellulose), and mixtures thereof. See, e.g., Ramirez-Farias et al., Br J Nutr (2008) 4:1-10; Pool-Zobel and Sauer, J Nutr (2007), 137:25805-25845, each of which are herein incorporated by reference in their entirety for all intended purposes.

The term “status epilepticus” or “SE” refers to seizures continuing for longer than 5 minutes if convulsive, and greater than 10 minutes if associated with loss of awareness but no convulsions. In certain embodiments, “status epilepticus” does not include those subjects with epilepsia partialis continua.

The term “encephalopathy” refers to an alteration in mental status.

The term “epilepsia partialis continua” refers a form of continuous seizures which manifests with continuous twitching of a body part without alteration in awareness.

The term “NORSE” refers to new onset refractory status epilepticus, an entity in which status epilepticus is refractory to first (i.e. appropriately dosed benzodiazepine) and second line (i.e. antiseizure medication such as phenytoin, valproic acid, lacosamide, levetiracetam or phenobarbital) therapy, of which no known cause is initially identified despite standard neuroimaging, infectious, and metabolic testing. NORSE is generally thought to be a disorder primarily caused by neuroinflammation. In certain embodiments, “NORSE” does not include those subjects with epilepsia partialis continua.

The term “chronic epilepsy patients” refers to patients suffering from chronic epilepsy who have not experienced status epilepticus and are undergoing elective admission to the hospital for the purposes of planned diagnostic investigations (video-EEG), and without relevant comorbidities that can affect gut microbiome. In certain embodiments, the chronic epilepsy patients are otherwise healthy.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “therapeutically effective amount” refers to the amount of a bacterial inoculant, fungal inoculant, a compound (e.g., a prebiotic, a probiotic, antibiotic, or an antifungal compound), or a composition that, when administered to a subject for treating (e.g., preventing or ameliorating) a state, disorder or condition, is sufficient to effect such treatment. The “therapeutically effective amount” will vary depending, e.g., on the compound, composition, bacteria, fungus, or analogues administered as well as the disease, its severity, and physical conditions and responsiveness of the subject to be treated.

As used herein, the phrase “pharmaceutically acceptable” refers to molecular entities and compositions that are generally regarded as physiologically tolerable.

As used herein, the term “combination” of a bacterial inoculant, fungal inoculant, probiotic, analogue, prebiotic compound, antibiotic, or an antifungal compound and at least a second pharmaceutically active ingredient means at least two, but any desired combination of compounds can be delivered simultaneously or sequentially (e.g., within a 24 hour period).

Within the meaning of the present invention, the term “conjoint administration” is used to refer to administration of a probiotic and a prebiotic simultaneously in one composition, or simultaneously in different compositions, or sequentially (preferably, within a 24 hour period).

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

As used herein, the term “stimulate” when used in connection with growth and/or activity of microorganisms (e.g., fungi or bacteria) encompasses the term “enhance”.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437, each of which are herein incorporated by reference in their entirety for all intended purposes.

### Diagnostic Methods of SE and NORSE

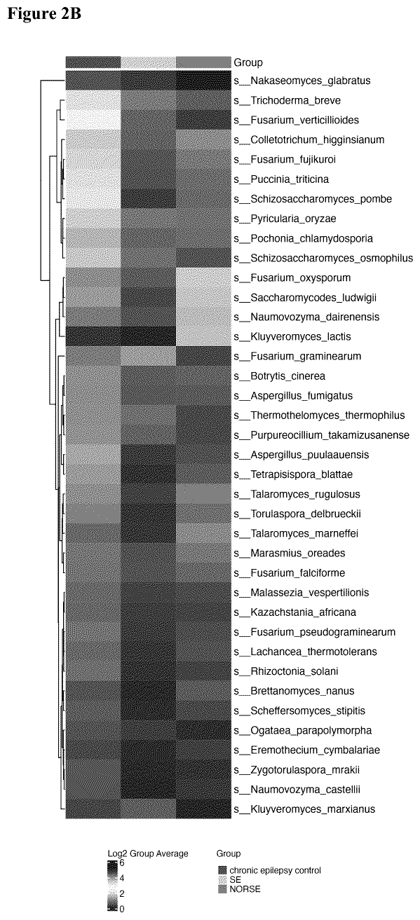

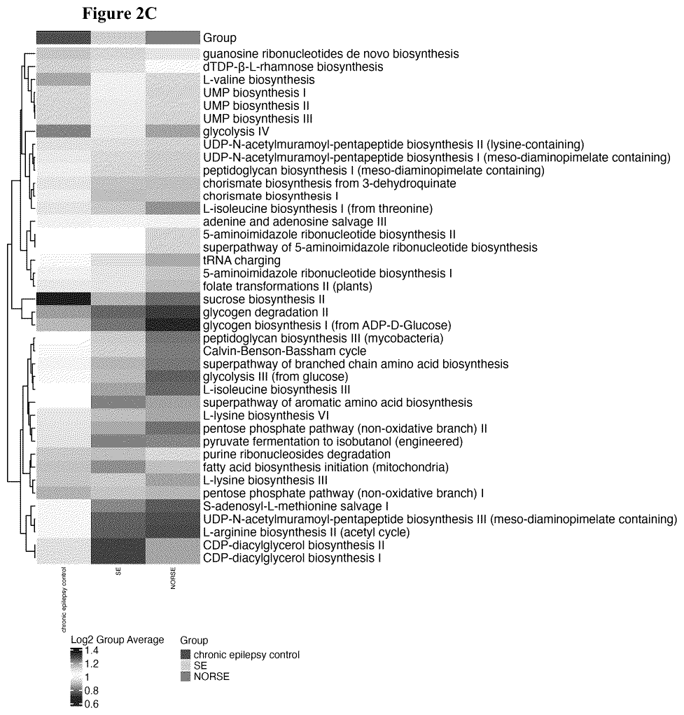

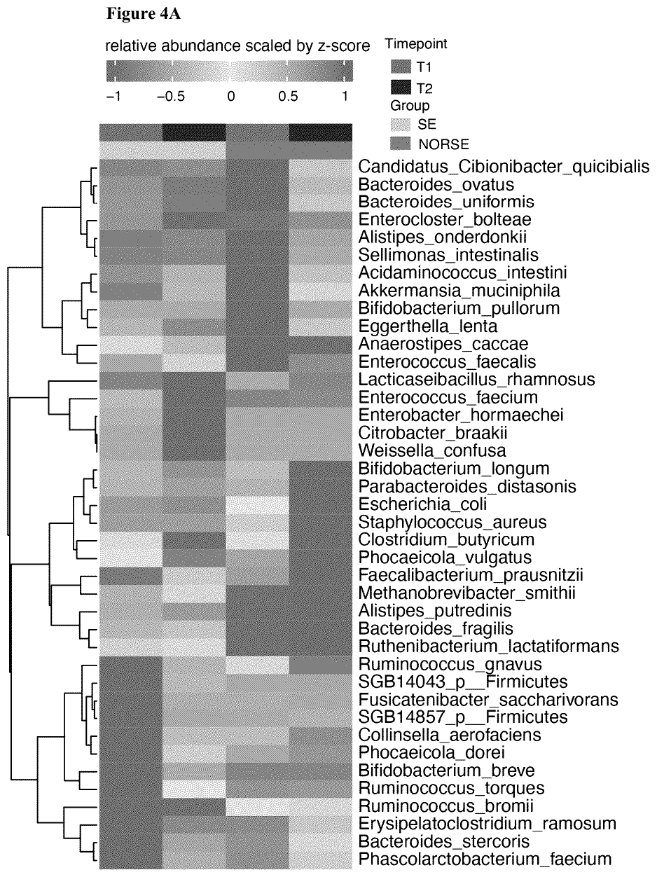

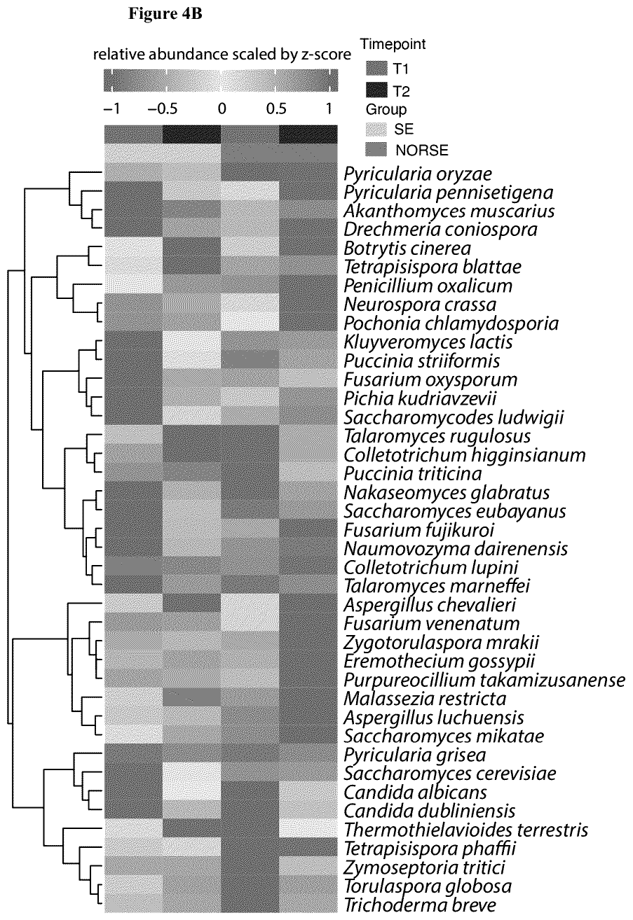

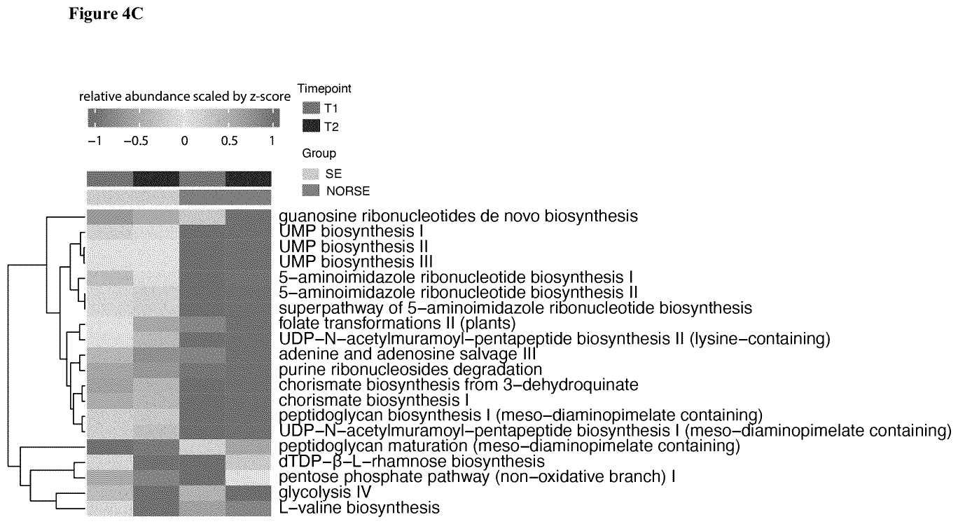

In one aspect, the present disclosure provides a method for the diagnosis of a subject suffering from Status Epilepticus (SE) or at risk of suffering from SE, said method comprising (a) determining the level of at least one strain of the fungi from FIGS. 2, 4, and/or 6 and/or at least one strain of the bacteria from FIGS. 2, 4, and/or 6 or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA in the gastrointestinal microbiota (e.g., fecal) of the subject and (b) comparing the level determined in step (a) to the level of the same fungi and/or bacteria in a control gastrointestinal microbiota (e.g., fecal microbiota), and (c) determining that the subject has SE or at risk of suffering from SE, wherein the level of at least one of the strains measured in step (a) is lower (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in controls and/or the level of at least one of the strains measured in step (a) is higher (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% higher) than in controls. In certain embodiments, subjects with SE exclude those with epilepsia partialis continua (EPC).

In one aspect, the present disclosure provides a method for in the diagnosis of a subject suffering from NORSE or at risk of suffering from NORSE, said method comprising (a) determining the level of at least one strain of the fungi from FIGS. 2, 4, and/or 6 and/or at least one strain of the bacteria from FIGS. 2, 4, and/or 6 or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA in the gastrointestinal microbiota (e.g., fecal) of the subject and (b) comparing the level determined in step (a) to the level of the same fungi and/or bacteria in a control gastrointestinal microbiota (e.g., fecal), and (c) determining that the subject has NORSE or at risk of suffering from NORSE, wherein the level of at least one of the strains measured in step (a) is lower (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in controls and/or the level of at least one of the strains measured in step (a) is higher (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% higher) than in controls. In certain embodiments, subjects with NORSE exclude those with epilepsia partialis continua (EPC).

In certain embodiments, the level of at least 2, 3, 4, 5, 6, 7, 8, 9, 10, 20, 50 or more strains from the taxa listed in FIGS. 2, 4, 6, 9, and/or 10 or Table 1 is determined. In certain embodiments, the level of at least 2, 3, 4, 5, 6, 7, 8, 9, 10, 20, 50 or more strains from the taxa listed in FIGS. 2, 4, 6, 9, and/or 10 or Table 1 is determined and compared to the same fungi and/or bacteria in a control. Table 1 is a non-exhaustive list of fungi, bacteria, and pathways that are enriched or depleted in SE and/or NORSE patients as compared to control chronic epilepsy patients.

TABLE 1
Species observed to be enriched or depleted in fecal microbiomes
from Status Epilepticus (SE) or NORSE patients when
compared to otherwise healthy chronic epilepsy patients. If left
blank, no discernable abundance pattern is observed.

| | SE | NORSE |
|---|---|---|
| Bacteria | | |
| Enterococcus faecalis | Enriched | Enriched |
| Enterococcus faecium | | Enriched |
| Enterocloster bolteae | Enriched | Enriched |
| Escherichia coli | Enriched | Enriched |
| Sellimonas intestinalis | Enriched | |
| Akkermansia muciniphila | Enriched | |
| Lacticaseibacillus rhamnosus | Enriched | |
| Lacticaseibacillus paracasei | | Enriched |
| Clostridium leptum | Depleted | |
| Streptococcus anginosus | Enriched | |
| Roseburia hominis | | |
| Bacteroides stercoris | Depleted | |
| Fusicatenibacter saccharivorans | Depleted | |
| Eubacterium rectale | Depleted | Depleted |
| Fungi | | |
| Saccharomycetales | Enriched | |
| Nakaseomyces glabratus | | Enriched |
| Marasmius oreades | | Enriched |
| Trichoderma breve | Depleted | Depleted |
| Fusarium verticillioides | Depleted | Depleted |
| MetaCyc Pathways | | |
| Guanosine ribonucleotides de novo | | Depleted |
| biosynthesis | | |
| dTDP-β-L-rhamnose biosynthesis | Depleted | |
| L-valine biosynthesis | Depleted | Depleted |
| UMP biosynthesis (I, II, III) | Depleted | Depleted |
| Glycolysis IV | Depleted | Depleted |
| Sucrose biosynthesis II | Depleted | Depleted |
| Glycogen degradation II | Depleted | Depleted |
| Glycogen biosynthesis I | Depleted | Depleted |

In certain embodiments, the presence of at least one strain of Enterococcus, Enterocloster, Escherichia, Sellimonas, Akkermansia, Lacticaseibacillus, Streptococcus, or Roseburia or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of SE or a subject at risk of suffering from SE. In certain embodiments, the at least one strain of Enterococcus, Enterocloster, Escherichia, Sellimonas, Akkermansia, Lacticaseibacillus, Streptococcus, or Roseburia or a closely related OTU as outlined above is found in the GI microbiota (e.g., cecal, ileal, colonic, and fecal microbiota). In certain embodiments, the at least one strain of Enterococcus faecalis, Enterococcus, Enterocloster, Escherichia, Sellimonas, Akkermansia, Lacticaseibacillus, Streptococcus, or Roseburia or a closely related OTU as outlined above is found in fecal microbiota.

In certain embodiments, the presence of at least one strain of Enterococcus faecalis, Enterocloster bolteae, Escherichia coli, Sellimonas intestinalis, Akkermansia muciniphila, Lacticaseibacillus rhamnosus, Streptococcus anginosus, or Roseburia hominis or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of SE or a subject at risk of suffering from SE. In certain embodiments, the at least one strain of Enterococcus faecalis, Enterocloster bolteae, Escherichia coli, Sellimonas intestinalis, Akkermansia muciniphila, Lacticaseibacillus rhamnosus, Streptococcus anginosus, or Roseburia hominis or a closely related OTU as outlined above is found in the GI microbiota (e.g., cecal, ileal, colonic, and fecal microbiota). In certain embodiments, the at least one strain of Enterococcus faecalis, Enterocloster bolteae, Escherichia coli, Sellimonas intestinalis, Akkermansia muciniphila, Lacticaseibacillus rhamnosus, Streptococcus anginosus, or Roseburia hominis or a closely related OTU as outlined above is found in fecal microbiota.

In certain embodiments, the absence or decreased levels of at least one strain of Trichoderma, Fusarium, Clostridium, Bacteroides, Fusicatenibacter, or Eubacterium or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of SE or a subject at risk of suffering from SE. In certain embodiments, the at least one strain of Trichoderma, Fusarium, Clostridium, Bacteroides, Fusicatenibacter, or Eubacterium or a closely related OTU as outlined above is absent or decreased in the GI microbiota (e.g., cecal, ileal, colonic, and fecal microbiota). In certain embodiments, the at least one strain of Trichoderma, Fusarium, Clostridium, Bacteroides, Fusicatenibacter, or Eubacterium is absent or decreased in fecal microbiota.

In certain embodiments, the absence or decreased levels of at least one strain of Trichoderma breve, Fusarium verticillioides, Clostridium leptum, Bacteroides stercoris, Fusicatenibacter saccharivorans, or Eubacterium rectale or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of SE or a subject at risk of suffering from SE. In certain embodiments, the at least one strain of Trichoderma breve, Fusarium verticillioides, Clostridium leptum, Bacteroides stercoris, Fusicatenibacter saccharivorans, or Eubacterium rectale or a closely related OTU as outlined above is absent or decreased in the GI microbiota (e.g., cecal, ileal, colonic, and fecal microbiota). In certain embodiments, the at least one strain of Trichoderma breve, Fusarium verticillioides, Clostridium leptum, Bacteroides stercoris, Fusicatenibacter saccharivorans, or Eubacterium rectale is absent or decreased in fecal microbiota.

In certain embodiments, the presence of at least one strain of Nakaseomyces, Marasmius, Enterococcus, Escherichia, Enterocloster, or Lacticaseibacillus or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of NORSE or a subject at risk of suffering from NORSE. In certain embodiments, the at least one strain of Nakaseomyces, Marasmius, Enterococcus, Escherichia, Enterocloster, or Lacticaseibacillus or a closely related OTU as outlined above is found in the GI microbiota (e.g., cecal, ileal, colonic, and fecal microbiota). In certain embodiments, the at least one strain of Nakaseomyces, Marasmius, Enterococcus, Escherichia, Enterocloster, or Lacticaseibacillus or a closely related OTU as outlined above is found in fecal microbiota.

In certain embodiments, the presence of at least one strain of Nakaseomyces glabratus, Marasmius oreades, Enterococcus faecalis, Enterococcus faecium, Escherichia coli, Enterocloster bolteae, Lacticaseibacillus paracasei, or Clostridiaceae_bacterium_OM02_2AC species or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of NORSE or a subject at risk of suffering from NORSE. In certain embodiments, the at least one strain of Nakaseomyces glabratus, Marasmius oreades, Enterococcus faecalis, Enterococcus faecium, Escherichia coli, Enterocloster bolteae, Lacticaseibacillus paracasei, or Clostridiaceae_bacterium_OM02_2AC species or a closely related OTU as outlined above is found in the GI microbiota (e.g., cecal, ileal, colonic, and fecal microbiota). In certain embodiments, the at least one strain of Nakaseomyces glabratus, Marasmius oreades, Enterococcus faecalis, Enterococcus faecium, Escherichia coli, Enterocloster bolteae, Lacticaseibacillus paracasei, or Clostridiaceae_bacterium_OM02_2AC species or a closely related OTU as outlined above is found in fecal microbiota.

In certain embodiments, the absence or decreased levels of at least one strain of Trichoderma, Fusarium, or Eubacterium or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of NORSE or a subject at risk of suffering from NORSE. In certain embodiments, the at least one strain of Trichoderma, Fusarium, or Eubacterium or a closely related OTU as outlined above is absent or decreased in the GI microbiota (e.g., cecal, ileal, colonic, and fecal microbiota). In certain embodiments, the at least one strain of Trichoderma, Fusarium, or Eubacterium or a closely related OTU as outlined above is absent or decreased in fecal microbiota.

In certain embodiments, the absence or decreased levels of at least one strain of Trichoderma breve, Fusarium verticillioides, or Eubacterium rectale or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative of NORSE or a subject at risk of suffering from NORSE. In certain embodiments, the at least one strain of Trichoderma breve, Fusarium verticillioides, or Eubacterium rectale or a closely related OTU as outlined above is absent or decreased in the GI microbiota (e.g., cecal, ileal, colonic, and fecal microbiota). In certain embodiments, the at least one strain of Trichoderma breve, Fusarium verticillioides, or Eubacterium rectale or a closely related OTU as outlined above is absent or decreased in fecal microbiota.

In one aspect, the present disclosure provides a method for the diagnosis of a subject suffering from SE or at risk of suffering from SE, said method comprising (a) determining in the fecal microbiota sample isolated from the subject the level of at least one strain of fungi from Saccharomycetales or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Enterococcus faecalis, Enterocloster bolteae, Escherichia coli, Sellimonas intestinalis, Akkermansia muciniphila, Lacticaseibacillus rhamnosus, Streptococcus anginosus, and Roseburia hominis or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level of the same fungi and/or bacteria in the control fecal microbiota, and (c) identifying that the subject has SE or at risk of suffering from SE, wherein the level of at least one of the strains determined in step (a) is higher than in the control. In certain embodiments, the level of at least one of the strains determined in step (a) is at least 2-log-fold higher, 3-log-fold higher, 4-log-fold higher, 5-log-fold higher, 6-log-fold higher, 7-log-fold higher, 8-log-fold higher, 9-log-fold higher, or 10-log-fold higher. In certain embodiments, the level of at least one of the strains determined in step (a) is at least 2-log-fold higher. In certain embodiments, the level of at least one of the strains determined in step (a) is at least 5-log-fold higher. In certain embodiments, the level of at least one of the strains determined in step (a) is at least 6-log-fold higher.

In one aspect, the present disclosure provides a method for the diagnosis of a subject suffering from SE or at risk of suffering from SE, said method comprising (a) determining in a fecal microbiota sample isolated from the subject the level of at least one strain of fungi from one or more species selected from Trichoderma breve and Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single ITS1 or ITS2 region of the 18S rRNA and/or the level of at least one strain of bacteria from one or more species selected from Clostridium leptum, Bacteroides stercoris, Fusicatenibacter saccharivorans, and Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA and (b) comparing the level(s) determined in step (a) to the level of the same fungi and/or bacteria in the control fecal microbiota, and (c) identifying that the subject has SE or at risk of suffering from SE, wherein the level of at least one of the strains determined in step (a) is lower than in the control. In certain embodiments, the level of at least one of the strains determined in step (a) is at least 2-log-fold lower, 3-log-fold lower, 4-log-fold lower, 5-log-fold lower, 6-log-fold lower, 7-log-fold lower, 8-log-fold lower, 9-log-fold lower, or 10-log-fold lower. In certain embodiments, the level of at least one of the strains determined in step (a) is at least 2-log-fold lower.

In one aspect, the present disclosure provides a method for the diagnosis of a subject suffering from NORSE or at risk of suffering from NORSE, said method comprising (a) determining in the fecal microbiota sample isolated from the subject the level of at least one strain of fungi from genus Nakaseomyces glabratus or Marasmius oreades or a closely related OTU which has at least 90% (or at least 95%, or at least 97%, or at least 99%) sequence identity to 18S rRNA over its entire length or has at least 90% (or at least 95%, or at least 99%) sequence identity to any single ITS1 or ITS2 region of the 18S rRNA and/or the level of at least one strain of bacteria from one or more species selected from Enterococcus faecalis, Enterococcus faecium, Escherichia coli, Enterocloster bolteae, Lacticaseibacillus paracasei, and Clostridiaceae_bacterium_OM02_2AC species or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level of the same fungi in the control fecal microbiota, and (c) identifying that the subject has NORSE or at risk of suffering from NORSE, wherein the level of at least one of the strains determined in step (a) is higher than in the control. In certain embodiments, the level of at least one of the strains determined in step (a) is at least 2-log-fold higher, 3-log-fold higher, 4-log-fold higher, 5-log-fold higher, 6-log-fold higher, 7-log-fold higher, 8-log-fold higher, 9-log-fold higher, or 10-log-fold higher. In certain embodiments, the level of at least one of the strains determined in step (a) is at least 2-log-fold higher. In certain embodiments, the level of at least one of the strains determined in step (a) is at least 5-log-fold higher. In certain embodiments, the level of at least one of the strains determined in step (a) is at least 6-log-fold higher.

In one aspect, the present disclosure provides a method for the diagnosis of a subject suffering from NORSE or at risk of suffering from NORSE, said method comprising (a) determining in the fecal microbiota sample isolated from the subject the level of at least one strain of fungi from one or more species selected from Trichoderma breve and Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single ITS1 or ITS2 region of the 18S rRNA and/or the level of at least one strain of bacteria from one or more species from Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level of the same fungi in the control fecal microbiota, and (c) identifying that the subject has NORSE or at risk of suffering from NORSE, wherein the level of at least one of the strains determined in step (a) is lower than in the control. In certain embodiments, the level of at least one of the strains determined in step (a) is at least 2-log-fold lower, 3-log-fold lower, 4-log-fold lower, 5-log-fold lower, 6-log-fold lower, 7-log-fold lower, 8-log-fold lower, 9-log-fold lower, or 10-log-fold lower. In certain embodiments, the level of at least one of the strains determined in step (a) is at least 2-log-fold lower.

In certain embodiments, the GI microbiota sample can be taken from the stomach, duodenum, jejunum, ileum, cecum, colon, and feces. In certain embodiments, the GI microbiota sample can be taken from the feces or intestines.

In one aspect, the present disclosure provides a method for the diagnosis of a subject suffering from SE or at risk of suffering from SE, said method comprising (a) determining in a fecal microbiota sample isolated from the subject the activity level of at least one pathway selected from dTDP-β-L-rhamnose biosynthesis, L-valine biosynthesis, UMP biosynthesis (I, II, III), Glycolysis IV, Sucrose biosynthesis II, Glycogen degradation II, and Glycogen biosynthesis I and (b) comparing the level(s) determined in step (a) to the level of the same pathway in the control fecal microbiota, and (c) identifying that the subject has SE or at risk of suffering from SE, wherein the level of at least one of the pathways determined in step (a) is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in the control.

In certain embodiments, the subject has SE or is likely to develop SE when the activity level of at least one pathway selected from dTDP-β-L-rhamnose biosynthesis, L-valine biosynthesis, UMP biosynthesis (I, II, III), Glycolysis IV, Sucrose biosynthesis II, Glycogen degradation II, and Glycogen biosynthesis I in the fecal microbiota sample is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in a control sample. In certain embodiments, the accuracy of the diagnosis of SE is improved when the activity level of at least one pathway selected from dTDP-β-L-rhamnose biosynthesis, L-valine biosynthesis, UMP biosynthesis (I, II, III), Glycolysis IV, Sucrose biosynthesis II, Glycogen degradation II, and Glycogen biosynthesis I in the fecal microbiota sample is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in a control sample.

In one aspect, the present disclosure provides a method for the diagnosis of a subject suffering from NORSE or at risk of suffering from NORSE, said method comprising (a) determining in a fecal microbiota sample isolated from the subject the activity level of at least one pathway selected from Guanosine ribonucleotides de novo biosynthesis, L-valine biosynthesis, UMP biosynthesis (I, II, III), Glycolysis IV, Sucrose biosynthesis II, Glycogen degradation II, and Glycogen biosynthesis I and (b) comparing the level(s) determined in step (a) to the level of the same pathway in the control fecal microbiota, and (c) identifying that the subject has SE or at risk of suffering from SE, wherein the level of at least one of the pathways determined in step (a) is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in the control.

In certain embodiments, the subject has NORSE or is likely to develop NORSE when the activity level of at least one pathway selected from Guanosine ribonucleotides de novo biosynthesis, L-valine biosynthesis, UMP biosynthesis (I, II, III), Glycolysis IV, Sucrose biosynthesis II, Glycogen degradation II, and Glycogen biosynthesis I in the fecal microbiota sample is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in a control sample. In certain embodiments, the accuracy of the diagnosis of NORSE is improved when the activity level of at least one pathway selected from Guanosine ribonucleotides de novo biosynthesis, L-valine biosynthesis, UMP biosynthesis (I, II, III), Glycolysis IV, Sucrose biosynthesis II, Glycogen degradation II, and Glycogen biosynthesis I in the fecal microbiota sample is lower (e.g., at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in a control sample.

In certain embodiments, the method further comprises determining the level of one or more cytokines in a blood sample from the subject.

In certain embodiments, the subject has SE or is likely to develop SE when the level of at least one of GCSF, IL1b, IL6, IL8, IL4, IL10, IL12p70, TNFa, IL17A, CCL2, MIP1a, and VEGF, in the blood sample is higher than in a control blood sample. In certain embodiments, the subject has SE or is likely to develop SE when the level of at least one of GCSF, IL10, IL12p70, IL1b, IL4, TNFa, and IL17A in the blood sample is higher than in a control blood sample.

In certain embodiments, the accuracy of the diagnosis of SE is improved when the level of at least one of GCSF, IL1b, IL6, IL8, IL4, IL10, IL12p70, TNFa, IL17A, CCL2, MIP1a, and VEGF in the blood sample is higher than in a control blood sample. In certain embodiments, the accuracy of the diagnosis of SE is improved when the level of at least one of GCSF, IL10, IL12p70, IL1b, IL4, TNFa, and IL17A in the blood sample is higher than in a control blood sample.

In certain embodiments, the subject has NORSE or is likely to develop NORSE when the level of at least one of IL6, CCL2, GCSF, IL1b, MIP1a, IL10, IL12p70, IL4, TNFa, and IL17A in the blood sample is higher than in the control blood sample. In certain embodiments, the subject has NORSE or is likely to develop NORSE when the level of at least one of IL6, CCL2, GCSF, and IL1b in the blood sample is higher than in the control blood sample.

In certain embodiments, the accuracy of the diagnosis of NORSE is improved when the level of at least one of IL6, CCL2, GCSF, IL1b, MIP1a, IL10, IL12p70, IL4, TNFa, and IL17A in the blood sample is higher than in the control blood sample. In certain embodiments, the accuracy of the diagnosis of NORSE is improved when the level of at least one of IL6, CCL2, GCSF, and IL1b in the blood sample is higher than in the control blood sample.

In certain embodiments, the control (e.g., control microbiota) is chronic epilepsy patients. In certain embodiments, the chronic epilepsy patients are otherwise healthy. In certain embodiments, rather than comparing with chronic epilepsy patients, the sample (e.g., fungi and/or bacteria level) is compared to an earlier sample taken from the same subject. The sample could be taken before or after treatment. The sample could be taken before or after symptoms of SE and/or NORSE. In certain embodiments, the control is age- and/or sex- and/or ethnicity-matched.

In certain embodiments, the average/mean is obtained by testing at least two, at least three, at least four, at least five, at least 10, at least 20, at least 25, at least 50, at least 75, or at least 100 control subjects. In certain embodiments, the average is the mean plus one, two, or three standard deviations of a group of control matched subjects. In certain embodiments, the abundance of fungi and/or bacteria is determined to be statistically significantly higher than the control abundance if the abundance is higher than the mean value of normal plus one standard deviation. In certain embodiments, the abundance of fungi and/or bacteria is determined to be statistically significantly higher than the control abundance if the abundance is higher than the mean value of normal plus two standard deviations. In certain embodiments, the abundance of the fungi and/or bacteria is determined to be statistically significantly higher than the control abundance if the abundance is higher than the mean value of normal plus three standard deviations. In certain embodiments, the abundance of the fungi and/or bacteria is determined to be statistically significantly higher than the control abundance if the abundance is higher than the mean value calculated for at least 40 matched control subjects plus three standard deviations.

In certain embodiments, the predetermined standard is a value which represents a statistically validated threshold ratio of the abundances of the fungi and/or bacteria equal to the highest possible value within the range of corresponding values in a large cohort of control subjects or in a large cohort of SE or NORSE patients.

Values will vary based on the methods for quantitation and should be normalized for the assay.

In certain embodiments of any of the diagnostic methods described above, the method further includes recruiting the subject in a clinical trial.

In certain embodiments of any of the diagnostic methods described above, the method further comprises administering a treatment to the subject. The treatment may comprise any of the treatment methods described below.

Non-limiting examples of the methods which can be used for determining the relative abundance of the fungi and/or bacterial strains include, for example, a method selected from the group consisting of quantitative polymerase chain reaction (qPCR), high-throughput sequencing, transcriptomic analysis, sequencing of bacterial 16S rRNA, sequencing of fungal 18S rRNA, shotgun metagenome sequencing, bacterial or fungal genotype pattern based fingerprinting (DNA fingerprinting), proteomic analysis, metabolomics analysis, and metagenomics (i.e., the approach of shearing DNA extracted from fecal samples and sequencing the small fragments to study not only the microbial species composition, but also the gene functions and metabolic pathways within them).

Additional methods include methods of evaluating the microbiome population in a subject or diagnosing an abnormal microbiome development. Methods include monitoring the subject's microbiome after the administration of the compound or composition that inhibits growth and/or activity of one or more strains of fungi and/or bacteria by: (a) determining a relative level of one or more strains of fungi and/or bacteria in a microbiome sample obtained from the subject, and (b) comparing the relative level(s) determined in step (a) to (i) a predetermined standard value or (ii) to the level(s) of the same taxa or genus in a control subject or (iii) to the average value of level of the same taxa or genus in several control subjects. The subject's sample may be isolated from feces, skin, intestines, intestinal mucosa, oral mucosa, conjunctive mucosa, or nasal mucosa. In certain embodiments, the subject's sample may be isolated from feces. It may be compared to a control subject.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ, each of which are herein incorporated by reference in their entirety for all intended purposes.

### Diagnostic Methods of Predicting Worse Outcomes

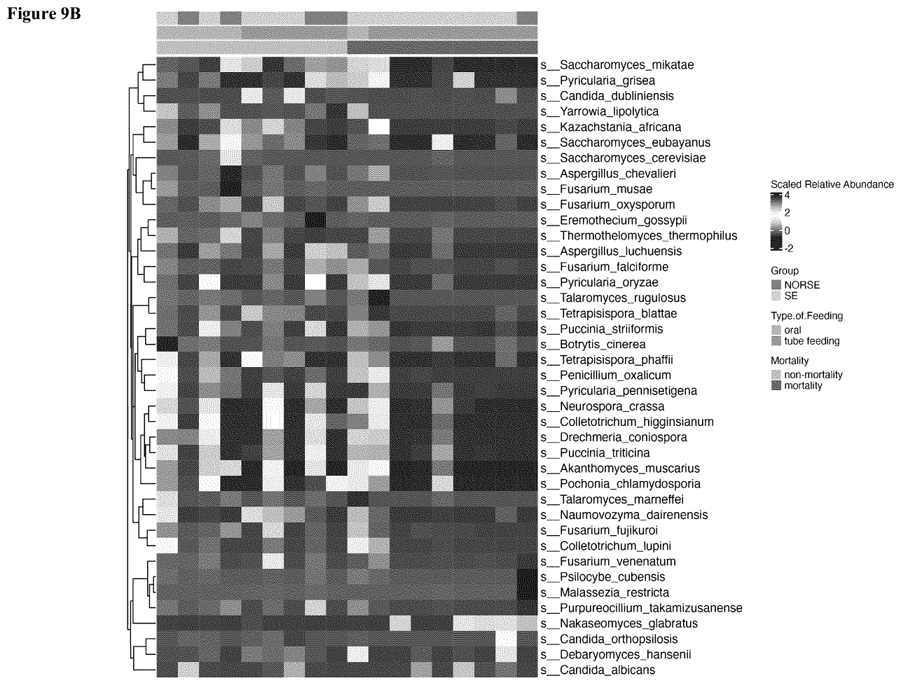

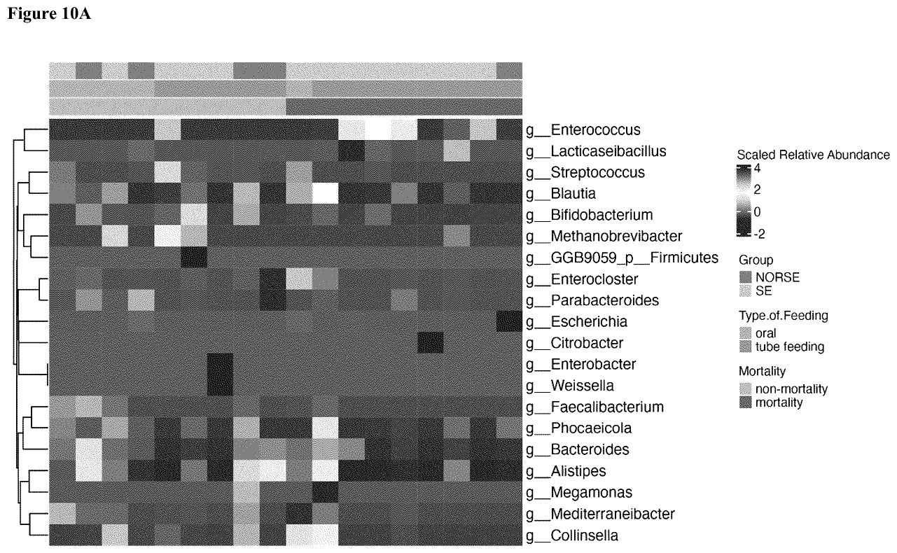

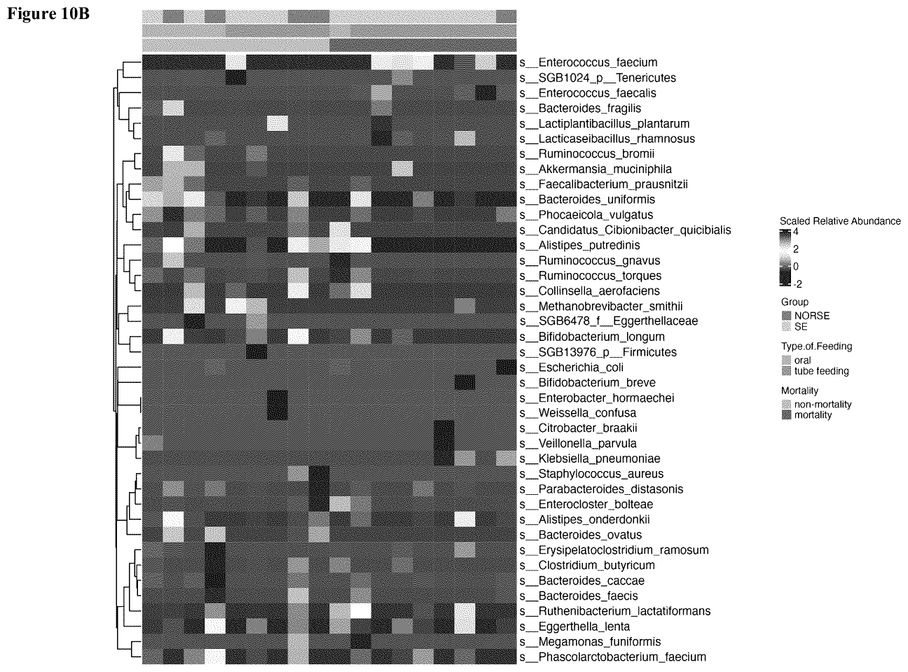

In one aspect, the present disclosure provides a method for predicting risk of or determining the likelihood of a worse outcome associated with SE and/or NORSE in a subject presenting with SE and/or NORSE, said method comprising (a) determining the level of at least one strain of the fungi from FIG. 9, FIG. 10, and/or Tables 8 and 9 and/at least one strain of bacteria from FIG. 9, FIG. 10, and/or Tables 8 and 9 or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA in the gastrointestinal microbiota (e.g., fecal) of the subject and (b) comparing the level determined in step (a) to the level of the same fungi and/or bacteria in a control gastrointestinal microbiota (e.g., fecal microbiota), and (c) determining that the subject is at risk of a worse outcome associated with SE and/or NORSE, wherein the level of at least one of the strains measured in step (a) is lower (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% lower) than in controls and/or the level of at least one of the strains measured in step (a) is higher (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% higher) than in controls. In certain embodiments, subjects exclude those with epilepsia partialis continua (EPC).

In certain embodiments, the level of at least 2, 3, 4, 5, 6, 7, 8, 9, 10, 20, 50 or more strains from the taxa listed in FIG. 9, FIG. 10, and/or Tables 8 and 9 is determined. In certain embodiments, the level of at least 2, 3, 4, 5, 6, 7, 8, 9, 10, 20, 50 or more strains from the taxa listed in FIG. 9, FIG. 10, and/or Tables 8 and 9 is determined and compared to the same fungi and/or bacteria in a control.

In certain embodiments, the presence of at least one strain selected from Family Saccharomycetaceae, Family Enterococcaceae, Genus Enterococcus, and Species Enterococcus faecalis or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative a subject at risk of suffering from a worse outcome associated with SE and/or NORSE in a subject presenting with SE and/or NORSE. In certain embodiments, the at least one strain selected from Family Saccharomycetaceae, Family Enterococcaceae, Genus Enterococcus, and Species Enterococcus faecalis or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA is found in the GI microbiota (e.g., cecal, ileal, colonic, and fecal microbiota). In certain embodiments, the at least one strain selected from Family Saccharomycetaceae, Family Enterococcaceae, Genus Enterococcus, and Species Enterococcus faecalis or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA is found in fecal microbiota.

In certain embodiments, the absence or decreased levels of at least one strain selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castellii, Species Ogataea parapolymorpha, Species Pyricularia pennisetigena, Species Saccharomyces kudriavzevii, Species Saccharomyces mikatae, Species Yarrowia lipolytica, Family Atopobiaceae, Family Bifidobacteriaceae, Family Clostridiaceae, Family FGB2982 c CFGB2982 p Firmicutes, Family Lachnospiraceae, Family Oscillospiraceae, Family Staphylococcaceae, Genus Bifidobacterium, Genus Blautia, Genus Coprococcus, Genus Dorea, Genus GGB9342 f FGB2982 c CFGB2982 p Firmicutes, Genus GGB9699 f Oscillospiraceae, Genus Staphylococcus, Genus Streptococcus, Species Bifidobacterium dentium, Species Blautia obeum, Species Dorea longicatena, Species SGB14306 g GGB9342 f FGB2982 c CFGB2982 p Firmicutes, and Species SGBI5216 g GGB9699 f Oscillospiraceae or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA can be indicative a subject at risk of suffering from a worse outcome associated with SE and/or NORSE in a subject presenting with SE and/or NORSE. In certain embodiments, the at least one strain selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castellii, Species Ogataea parapolymorpha, Species Pyricularia pennisetigena, Species Saccharomyces kudriavzevii, Species Saccharomyces mikatae, Species Yarrowia lipolytica, Family Atopobiaceae, Family Bifidobacteriaceae, Family Clostridiaceae, Family FGB2982 c CFGB2982 p Firmicutes, Family Lachnospiraceae, Family Oscillospiraceae, Family Staphylococcaceae, Genus Bifidobacterium, Genus Blautia, Genus Coprococcus, Genus Dorea, Genus GGB9342 f FGB2982 c CFGB2982 p Firmicutes, Genus GGB9699 f Oscillospiraceae, Genus Staphylococcus, Genus Streptococcus, Species Bifidobacterium dentium, Species Blautia obeum, Species Dorea longicatena, Species SGB14306 g GGB9342 f FGB2982 c CFGB2982 p Firmicutes, and Species SGB15216 g GGB9699f Oscillospiraceae or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA is found in the GI microbiota (e.g., cecal, ileal, colonic, and fecal microbiota). In certain embodiments, the at least one strain selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castellii, Species Ogataea parapolymorpha, Species Pyricularia pennisetigena, Species Saccharomyces kudriavzevii, Species Saccharomyces mikatae, Species Yarrowia lipolytica, Family Atopobiaceae, Family Bifidobacteriaceae, Family Clostridiaceae, Family FGB2982 c CFGB2982 p Firmicutes, Family Lachnospiraceae, Family Oscillospiraceae, Family Staphylococcaceae, Genus Bifidobacterium, Genus Blautia, Genus Coprococcus, Genus Dorea, Genus GGB9342 f FGB2982 c CFGB2982 p Firmicutes, Genus GGB9699 f Oscillospiraceae, Genus Staphylococcus, Genus Streptococcus, Species Bifidobacterium dentium, Species Blautia obeum, Species Dorea longicatena, Species SGB14306 g GGB9342 f FGB2982 c CFGB2982 p Firmicutes, and Species SGB15216 g GGB9699 f Oscillospiraceae or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA is found in fecal microbiota.

In one aspect, the present disclosure provides a method for predicting risk of mortality associated with SE or NORSE in subjects presenting with SE or NORSE, said method comprising (a) determining in a fecal microbiota sample isolated from the subject the level of at least one strain of fungi from one or more species selected from Family Saccharomycetoutae or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Enterococcaceae, Genus Enterococcus, and Species Enterococcus faecalis, or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and (b) comparing the level(s) determined in step (a) to the level(s) of the same fungi and/or bacteria in a control fecal microbiota, and (c) identifying that the subject is at risk of mortality associated with SE or NORSE, wherein the level of at least one of the strains determined in step (a) is higher than in the control. In certain embodiments, the level of at least one of the strains determined in step (a) is at least 2-log-fold higher, 3-log-fold higher, 4-log-fold higher, 5-log-fold higher, 6-log-fold higher, 7-log-fold higher, 8-log-fold higher, 9-log-fold higher, or 10-log-fold higher. In certain embodiments, the level of at least one of the strains determined in step (a) is at least 2-log-fold higher. In certain embodiments, the level of at least one of the strains determined in step (a) is at least 5-log-fold higher. In certain embodiments, the level of at least one of the strains determined in step (a) is at least 6-log-fold higher.

In one aspect, the present disclosure provides a method for predicting risk of mortality associated with SE or NORSE in subjects presenting with SE or NORSE, said method comprising (a) determining in a fecal microbiota sample isolated from the subject the level of at least one strain of fungi from one or more species selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castellii, Species Ogataea parapolymorpha, Species Pyricularia pennisetigena, Species Saccharomyces kudriavzevii, Species Saccharomyces mikatae, and Species Yarrowiaoutpolytica or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Enterococcaceae, Genus Enterococcus, and Species Enterococcus faecalis, or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA and (b) comparing the level(s) determined in step (a) to the level(s) of the same fungi and/or bacteria in a control fecal microbiota, and (c) identifying that the subject is at risk of mortality associated with SE or NORSE, wherein the level of at least one of the strains determined in step (a) is lower than in the control. In certain embodiments, the level of at least one of the strains determined in step (a) is at least 2-log-fold lower, 3-log-fold lower, 4-log-fold lower, 5-log-fold lower, 6-log-fold lower, 7-log-fold lower, 8-log-fold lower, 9-log-fold lower, or 10-log-fold lower. In certain embodiments, the level of at least one of the strains determined in step (a) is at least 2-log-fold lower.

In certain embodiments, the worse outcome can be mortality, disability, epilepsy, cognitive impairment, neuroinflammation, and/or neurodegeneration. In certain embodiments, the worse outcome can be mortality.

In certain embodiments, the GI microbiota sample can be taken from the stomach, duodenum, jejunum, ileum, cecum, colon, and feces. In certain embodiments, the GI microbiota sample can be taken from the feces or intestines.

In certain embodiments, the control (e.g., control microbiota) is subjects presenting with SE or NORSE that did not experience mortality. In certain embodiments, rather than comparing with subjects presenting with SE or NORSE that did not experience mortality, the sample (e.g., fungi and/or bacteria level) is compared to an earlier sample taken from the same subject. The sample could be taken before or after treatment. The sample could be taken before or after symptoms of SE and/or NORSE. In certain embodiments, the control is age- and/or sex- and/or ethnicity-matched.

In certain embodiments, the average/mean is obtained by testing at least two, at least three, at least four, at least five, at least 10, at least 20, at least 25, at least 50, at least 75, or at least 100 control subjects. In certain embodiments, the average is the mean plus one, two, or three standard deviations of a group of control matched subjects. In certain embodiments, the abundance of fungi and/or bacteria is determined to be statistically significantly higher than the control abundance if the abundance is higher than the mean value of normal plus one standard deviation. In certain embodiments, the abundance of fungi and/or bacteria is determined to be statistically significantly higher than the control abundance if the abundance is higher than the mean value of normal plus two standard deviations. In certain embodiments, the abundance of the fungi and/or bacteria is determined to be statistically significantly higher than the control abundance if the abundance is higher than the mean value of normal plus three standard deviations. In certain embodiments, the abundance of the fungi and/or bacteria is determined to be statistically significantly higher than the control abundance if the abundance is higher than the mean value calculated for at least 40 matched control subjects plus three standard deviations.

In certain embodiments, the predetermined standard is a value which represents a statistically validated threshold ratio of the abundances of the fungi and/or bacteria equal to the highest possible value within the range of corresponding values in a large cohort of control subjects.

Values will vary based on the methods for quantitation and should be normalized for the assay.

In certain embodiments of any of the diagnostic methods described above, the method further includes recruiting the subject in a clinical trial.

In certain embodiments of any of the diagnostic methods described above, the method further comprises administering a treatment to the subject. The treatment may comprise any of the treatment methods described below.

Non-limiting examples of the methods which can be used for determining the relative abundance of the fungi and/or bacterial strains include, for example, a method selected from the group consisting of quantitative polymerase chain reaction (qPCR), high-throughput sequencing, transcriptomic analysis, sequencing of bacterial 16S rRNA, sequencing of fungal 18S rRNA, shotgun metagenome sequencing, bacterial or fungal genotype pattern based fingerprinting (DNA fingerprinting), proteomic analysis, and metabolomics analysis.

Additional methods include methods of evaluating the microbiome population in a subject or diagnosing an abnormal microbiome development. Methods include monitoring the subject's microbiome after the administration of the compound or composition that inhibits growth and/or activity of one or more strains of fungi and/or bacteria by: (a) determining a relative level of one or more strains of fungi and/or bacteria in a microbiome sample obtained from the subject, and (b) comparing the relative level(s) determined in step (a) to (i) a predetermined standard value or (ii) to the level(s) of the same taxa or genus in a control subject or (iii) to the average value of level of the same taxa or genus in several control subjects. The subject's sample may be isolated from feces, skin, intestines, intestinal mucosa, oral mucosa, conjunctive mucosa, or nasal mucosa. In certain embodiments, the subject's sample may be isolated from feces. It may be compared to a control subject.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3^{rd} ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ, each of which are herein incorporated by reference in their entirety for all intended purposes.

### Therapeutic Methods

In one aspect, the invention provides a method for treating (including preventing) SE and/or NORSE in a subject in need thereof, said method comprising administering at least one compound and/or composition (e.g., a probiotic and/or a prebiotic composition), wherein the compound(s) or composition(s) stimulate growth and/or activity of one or more strains of fungi and/or bacteria or a closely related OTU which has at least 90% (or at least 95%, or at least 97%, or at least 99%) sequence identity to 16S rRNA for bacteria or 18S rRNA for fungus over its entire length or has at least 90% (or at least 95%, or at least 99%) sequence identity to any single V region of 16S rRNA or ITS1 or ITS2 region of the 18S rRNA. In certain embodiments, the compound or composition prevents a worse outcome associated with SE and/or NORSE. In certain embodiments, the worse outcome can be mortality, disability, epilepsy, cognitive impairment, neuroinflammation, and/or neurodegeneration. In certain embodiments, the worse outcome can be mortality.

In some embodiments, the probiotic comprises one or more strains of fungi from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In certain embodiments, the composition comprises at least 1, 2, 3, 4, 5, 6, 7, 8, 9, 10, 20, 50 or more strains from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In some embodiments, only nonpathogenic species within the taxa qualify for use in the probiotics or methods herein.

In some embodiments, the probiotic comprises one or more strains of bacteria from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In certain embodiments, the probiotic comprises at least 1, 2, 3, 4, 5, 6, 7, 8, 9, 10, 20, 50 or more strains from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In some embodiments, only nonpathogenic species within the taxa qualify for use in the probiotics or methods herein.

In some embodiments, the method comprises administering to the subject an effective amount of at least one compound or composition (e.g., a probiotic and/or a prebiotic composition), wherein said compound(s) and/or composition(s) stimulate growth and/or activity of one or more strains of fungi from the taxa listed in FIG. 2, 4, 6, or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal (GI) microbiota of the subject. In certain embodiments, the composition comprises at least 2, 3, 4, 5, 6, 7, 8, 9, 10, 20, 50 or more strains from the taxa listed in FIG. 2, 4, 6, or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In certain embodiments, the GI microbiota is selected from the group consisting of cecal, ileal, colonic, and fecal microbiota. In certain embodiments, the GI microbiota is fecal microbiota.

In some embodiments, the method comprises administering to the subject an effective amount of at least one compound or composition (e.g., a probiotic and/or a prebiotic composition), wherein said compound(s) and/or composition(s) stimulate growth and/or activity of one or more strains of bacteria from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal (GI) microbiota of the subject. In certain embodiments, the composition comprises at least 2, 3, 4, 5, 6, 7, 8, 9, 10, 20, 50 or more strains from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In certain embodiments, the GI microbiota is selected from the group consisting of cecal, ileal, colonic, and fecal microbiota. In certain embodiments, the GI microbiota is fecal microbiota.

In one aspect, the disclosure provides a method for treating (including preventing) SE in a subject in need thereof, said method comprising administering to the subject an effective amount of at least one compound or composition (e.g., a probiotic and/or a prebiotic composition), wherein the compound(s) or composition(s) stimulate the growth and/or activity of one or more strains of fungi selected from the genera Trichoderma and/or Fusarium or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of fungi from the genera Trichoderma and/or Fusarium or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) SE in a subject in need thereof, said method comprising administering to the subject an effective amount of at least one compound or composition (e.g., a probiotic and/or a prebiotic composition), wherein the compound(s) or composition(s) stimulate the growth and/or activity of one or more strains of fungi selected from the species Trichoderma breve and/or Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of fungi from the species Trichoderma breve and/or Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) SE in a subject in need thereof, said method comprising administering to the subject an effective amount of at least one compound or composition (e.g., a probiotic and/or a prebiotic composition), wherein the compound(s) or composition(s) stimulates the growth and/or activity of one or more strains of bacteria selected from the genera Clostridium, Bacteroides, Fusicatenibacter, and/or Eubacterium or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of bacteria from the species lostridium, Bacteroides, Fusicatenibacter, and/or Eubacteriumor a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) SE in a subject in need thereof, said method comprising administering to the subject an effective amount of at least one compound or composition (e.g., a probiotic and/or a prebiotic composition), wherein the compound(s) or composition(s) stimulates the growth and/or activity of one or more strains of bacteria selected from the species Clostridium leptum, Bacteroides stercoris, Fusicatenibacter saccharivorans, and/or Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of bacteria from the species Clostridium leptum, Bacteroides stercoris, Fusicatenibacter saccharivorans, and/or Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) NORSE in a subject in need thereof, said method comprising administering to the subject an effective amount of at least one compound or composition (e.g., a probiotic and/or a prebiotic composition), wherein the compound(s) or composition(s) stimulate the growth and/or activity of one or more strains of fungi selected from the genera Trichoderma and/or Fusarium or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of fungi from the species Trichoderma and/or Fusarium or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) NORSE in a subject in need thereof, said method comprising administering to the subject an effective amount of at least one compound or composition (e.g., a probiotic and/or a prebiotic composition), wherein the compound(s) or composition(s) stimulate the growth and/or activity of one or more strains of fungi selected from the species Trichoderma breve and/or Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of fungi from the species Trichoderma breve and/or Fusarium verticillioides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) NORSE in a subject in need thereof, said method comprising administering to the subject an effective amount of at least one compound or composition (e.g., a probiotic and/or a prebiotic composition), wherein the compound(s) or composition(s) stimulates the growth and/or activity of one or more strains of bacteria selected from the genera Eubacterium or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of bacteria from the genera Eubacterium or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) NORSE in a subject in need thereof, said method comprising administering to the subject an effective amount of at least one compound or composition (e.g., a probiotic and/or a prebiotic composition), wherein the compound(s) or composition(s) stimulates the growth and/or activity of one or more strains of bacteria selected from the species Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition stimulates the growth and/or activity of at least 2, 3, 4, 5, 6, 7, 8, 9, 10, 20, 50 or more strains of bacteria from the species Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) SE in a subject in need thereof, said method comprising administering to the subject an effective amount of at least one compound or composition, wherein the compound(s) or composition(s) inhibits the growth and/or activity of one or more strains of fungi selected from the order Saccharomycetales or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of fungi from the order Saccharomycetales or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) SE in a subject in need thereof, said method comprising administering to the subject an effective amount of at least one compound or composition, wherein the compound(s) or composition(s) inhibits the growth and/or activity of one or more strains of bacteria selected from the genera Enterococcus, Enterocloster, Escherichia, Sellimonas, Akkermansia, Fusicatenibacter, Lacticaseibacillus, Streptococcus, and Roseburia or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of bacteria from the species Enterococcus, Enterocloster, Escherichia, Sellimonas, Akkermansia, Fusicatenibacter, Lacticaseibacillus, Streptococcus, and Roseburia or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) SE in a subject in need thereof, said method comprising administering to the subject an effective amount of at least one compound or composition, wherein the compound(s) or composition(s) inhibits the growth and/or activity of one or more strains of bacteria selected from the species Enterococcus faecalis, Enterocloster bolteae, Escherichia coli, Sellimonas intestinalis, Akkermansia muciniphila, Fusicatenibacter saccharivorans, Lacticaseibacillus rhamnosus, Streptococcus anginosus, and Roseburia hominis or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of bacteria from the species Enterococcus faecalis, Enterocloster bolteae, Escherichia coli, Sellimonas intestinalis, Akkermansia muciniphila, Fusicatenibacter saccharivorans, Lacticaseibacillus rhamnosus, Streptococcus anginosus, and Roseburia hominis or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) NORSE in a subject in need thereof, said method comprising administering to the subject an effective amount of at least one compound or composition, wherein the compound(s) or composition(s) inhibits the growth and/or activity of one or more strains of fungi selected from the genera Nakaseomyces and Marasmius or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of fungi from the genera Nakaseomyces and Marasmius or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) NORSE in a subject in need thereof, said method comprising administering to the subject an effective amount of at least one compound or composition, wherein the compound(s) or composition(s) inhibits the growth and/or activity of one or more strains of fungi selected from the species Nakaseomyces glabratus and Marasmius oreades or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of fungi from the species Nakaseomyces glabratus and Marasmius oreades or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA.

In one aspect, the disclosure provides a method for treating (including preventing) NORSE in a subject in need thereof, said method comprising administering to the subject an effective amount of at least one compound or composition, wherein the compound(s) or composition(s) inhibits the growth and/or activity of one or more strains of bacteria selected from the genera Enterococcus, Enterococcus, Enterocloster, Escherichia, and Lacticaseibacillus or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of bacteria from the species Enterococcus, Enterococcus, Enterocloster, Escherichia, and Lacticaseibacillus or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) NORSE in a subject in need thereof, said method comprising administering to the subject an effective amount of at least one compound or composition, wherein the compound(s) or composition(s) inhibits the growth and/or activity of one or more strains of bacteria selected from the species Enterococcus faecalis, Enterococcus faecium, Enterocloster bolteae, Escherichia coli, and Lacticaseibacillus paracasei or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA in the gastrointestinal microbiota of the subject. In certain embodiments, the compound or composition inhibits the growth and/or activity of at least 2, 3, 4, 5, 6, 7, 8, 9, 10, 20, 50 or more strains of bacteria from the species Enterococcus faecalis, Enterococcus faecium, Enterocloster bolteae, Escherichia coli, and Lacticaseibacillus paracasei or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA.

In one aspect, the disclosure provides a method for treating (including preventing) SE in a subject in need thereof, said method comprising administering to the subject an effective amount of at least one compound or composition, wherein the compound(s) or composition(s) stimulates the activity of at least one pathway selected from dTDP-β-L-rhamnose biosynthesis, L-valine biosynthesis, UMP biosynthesis (I, II, III), Glycolysis IV, Sucrose biosynthesis II, Glycogen degradation II, and Glycogen biosynthesis I in the gastrointestinal microbiota of the subject. In certain embodiments, the at least one pathway is increased by at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

In one aspect, the disclosure provides a method for treating (including preventing) NORSE in a subject in need thereof, said method comprising administering to the subject an effective amount of at least one compound or composition, wherein the compound(s) or composition(s) stimulates the activity of at least one pathway selected from Guanosine ribonucleotides de novo biosynthesis, L-valine biosynthesis, UMP biosynthesis (I, II, III), Glycolysis IV, Sucrose biosynthesis II, Glycogen degradation II, and Glycogen biosynthesis I in the gastrointestinal microbiota of the subject. In certain embodiments, the at least one pathway is increased by at least 1%, is at least 2%, is at least 3%, is at least 4%, is at least 5%, is at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

In some embodiments, the method of treatment or prevention can be ablation or repopulation of one or more strains of bacterial or fungi or both. In some embodiments, the method of treatment or prevention can involve modulating the bacterial or fungal gene products (metabolites) based on gene functions of bacteria and fungi.

In some embodiments of any of the above methods involving administration of a probiotic composition, said probiotic composition comprises one or more OTUs which are independently characterized by, i.e., at least 95%, 96%, 97%, 98%, 99% or including 100% sequence identity to 16S rRNA sequences of the bacteria recited in FIG. 2, 4,6, 9, or 10 or Table 1. In another embodiment, the OTUs may be characterized by one or more of the variable regions of the 16S rRNA sequence (V1-V9). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (See, e.g., Brosius et al., Complete nucleotide sequence of a 16S ribosomal RNA gene from Escherichia coli, PNAS 75(10):4801-4805 (1978)). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU.

In certain embodiments, the growth is inhibited to the extent that the fungal strains are removed from the gastrointestinal microbiota (i.e., reduced or ablated).

In certain embodiments, the compound or composition comprises an antifungal compound or a natural product that inhibits fungal growth.

In certain embodiments, inhibiting growth and/or activity of at least one fungi species in the gastrointestinal microbiota according to any of the above methods involving such inhibition can be achieved, e.g., by administering an antifungal compound. In certain embodiments, the antifungal compound is administered in a therapeutic dose. In certain embodiments, the antifungal compound is administered in a sub-therapeutic dose.

Antifungal compounds useful in the methods and/or compositions of the invention include, but are not limited to, echocandin compounds (e.g., micafungin, caspofungin, cilofungin, and anidulafungin), triazole compounds (e.g., fluconazole, itraconazole, voriconazole, hexaconazole, isavuconazole, posaconazole, and ketoconazole), polyene compounds (e.g., amphotericin B, nystatin, and natamycin), and any combinations thereof.

Additional non-limiting examples of antifungal compound useful in the methods and/or compositions of the invention include flucytosine, azoles and echinocandins, and include specific compounds voriconazole, fluconazole, terbinafme, caspofungin, natamycin, amphotericin (e.g., amphotericin B), 5-FC, micafungin, anidulafungin, clotrimazole, isavuconazonium, itraconazole, flucytosine, griseofulvin, posaconazole, APX001, AR-12, ASP2397, Efungumab, F901318, MGCD290, and T-2307.

Natural products that inhibit fungal growth useful in the methods and/or compositions of the invention include, but are not limited to, citronella, naftifine and terbinafine.

In certain embodiments, the antifungal compound is amphotericin B or fluconazole.

In certain embodiments, the antifungal or antibiotic compound is administered from about 0.05 mg/ml to about 10 mg/ml per day. In certain embodiments, the antifungal compound is administered from about 0.075 mg/ml to about 8 mg/ml per day, about 0.1 mg/ml to about 6 mg/ml per day, about 0.25 mg/ml to about 5 mg/ml per day, about 0.5 mg/ml to about 4 mg/ml per day, about 0.75 mg/ml to about 3 mg/ml per day, or about 1 mg/ml to about 2 mg/ml per day. In certain embodiments, the antifungal or antibiotic compound is administered from about 0.5 mg/ml to about 1 mg/ml per day. In certain embodiments, the antifungal or antibiotic compound is administered at about 0.05 mg/ml, about 0.075 mg/ml, about 0.1 mg/ml, about 0.25 mg/ml, about 0.5 mg/ml, about 0.6 mg/ml, about 0.75 mg/ml, about 0.8 mg/ml, about 1.0 mg/ml, about 1.25 mg/ml, about 1.5 mg/ml, about 1.75 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, or about 10 mg/ml per day. In certain embodiments, the antifungal or antibiotic compound is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days. In certain embodiments, the antifungal or antibiotic compound is administered consecutive days, every other day, every third day, every fourth day, or once a week.

In one specific embodiment, the method comprises administering amphotericin B at 1 mg/ml per day for five consecutive days. In another specific embodiment, the method comprises administering fluconazole at 0.5 mg/ml per day for three weeks.

Non-limiting examples of the methods which can be used for determining the level of fungi include, e.g., quantitative PCR (qPCR), high-throughput sequencing, transcriptomic analysis, bacterial genotype pattern based fingerprinting (DNA fingerprinting), and proteomic analysis.

In one aspect, the disclosure provides a method for treating (including preventing) SE or NORSE in a subject in need thereof, said method comprising administering a probiotic and/or a prebiotic composition, wherein the composition(s) stimulate growth and/or activity of one or more strains of bacteria.

In one embodiment of any of the above methods of the invention, the probiotic is administered to the subject by a route selected from the group consisting of oral, rectal (e.g., by enema), mucosal, sublingual, and via naso/oro-gastric gavage.

Within a given composition, different bacterial strains can be contained in equal amounts (even combination) or in various proportions (uneven combinations) needed for achieving the maximal biological activity. For example, in a bacterial composition with two bacterial strains, the strains may be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three bacterial strains, the ratio of strains may be chosen pairwise from ratios for bacterial compositions with two strains. For example, in a bacterial composition comprising bacterial strains A, B, and C, at least one of the ratios between strain A and B, the ratio between strain B and C, and the ratio between strain A and C may be chosen, independently, from the pairwise combinations above. In one specific embodiment, the invention encompasses administering two or more bacteria-containing compositions to the same subject. Such compositions can be administered simultaneously or sequentially.

In one embodiment of any of the above methods of the invention, the probiotic is administered in a therapeutically effective amount. The dosages of the microbiota inoculum and/or probiotic composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization, e.g. 10⁶, 10⁷, 10⁸, 10⁹, and 10¹⁰ CFU for example, can be administered in a single dose. Lower doses can also be effective, e.g., 10⁴, and 10⁵ CFU.

The probiotic composition useful in any of the above methods can comprise, without limitation, e.g., live bacterial cells, conditionally lethal bacterial cells, inactivated bacterial cells, killed bacterial cells, spores (e.g., germination-competent spores), recombinant carrier strains, cell extract, and bacterially-derived products (natural or synthetic bacterially-derived products such as, e.g., bacterial antigens or bacterial metabolic products).

Bacterial strains administered in probiotic compositions according to the methods of the present invention can comprise live bacteria. One or several different bacterial inoculants can be administered simultaneously or sequentially (including administering at different times). Such bacteria can be isolated from gastrointestinal (GI) microbiota and grown in culture. The present invention also comprises administering “bacterial analogues”, such as recombinant carrier strains expressing one or more heterologous genes derived from the relevant bacterial species. The use of such recombinant bacteria may allow the use of lower therapeutic amounts due to higher protein expression. Non-limiting examples of recombinant carrier strains useful in the methods of the present invention include E. coli and Lactobacillus, Bacteroides and Oxalobacter. Methods describing the use of bacteria for heterologous protein delivery are described, e.g., in U.S. Pat. No. 6,803,231.

In certain embodiments, the probiotic comprises a preparation of the GI microbiota of a healthy subject. A suitable donor might have no known infections or colonizations of disease associated microbes and viruses. A spouse or family method without evidence of disease might be suitable. It might be best to transfer a carefully selected collection or consortium of commensal bacteria, with or without pretreatment that would facilitate colonization and prevent recurrence of the disease-associated taxa (i.e., species and strain).

Methods for producing bacterial compositions of the invention may include three main processing steps, combined with one or more mixing steps. The steps are: organism banking, organism production, and preservation. For banking, the strains included in the bacterial compositions of the invention may be (1) isolated directly from a specimen or taken from a banked stock, (2) optionally cultured on a nutrient agar or broth that supports growth to generate viable biomass, and (3) the biomass optionally preserved in multiple aliquots in long-term storage. The bacterial suspension can be freeze-dried to a powder and titrated. After drying, the powder may be blended to an appropriate potency, and mixed with other cultures and/or a filler such as microcrystalline cellulose for consistency and ease of handling, and the bacterial composition formulated as provided herein.

In one embodiment of any of the above methods of the invention, the probiotic is delivered to the subject in a form of a suspension, a pill, a tablet, a capsule, or a suppository. In another embodiment, the probiotic is delivered to the subject in a form of a liquid, foam, cream, spray, powder, or gel. In yet another embodiment, the probiotic is delivered to the subject in a saline suspension for use in feeding tubes, transmission via nasogastric tube, or enema. If live bacteria are used, the carrier should preferably contain an ingredient that promotes viability of the bacteria during storage.

The formulation can include added ingredients to improve palatability, improve shelf-life, impart nutritional benefits, and the like. If a reproducible and measured dose is desired, the bacteria can be administered by a rumen cannula.

In one embodiment of any of the above methods of the invention, the bacterial inoculum is delivered to the subject in a form of a composition which comprises (i) a carrier and/or excipient and/or (ii) one or more prebiotic agents which stimulate growth and/or activity of one or more bacteria present in the composition. In one specific embodiment, said composition comprises an excipient or a carrier that optimizes the seeding of the transferred microbiota.

In one embodiment of any of the above methods involving administration of a probiotic composition, said probiotic composition is reconstituted from a lyophilized preparation. In one embodiment of any of the above methods involving administration of a probiotic composition, said probiotic composition comprises a buffering agent to adjust pH.

In one embodiment, the probiotic composition comprises a buffering agent (e.g., sodium bicarbonate, infant formula, or other agents which allow bacteria to survive and grow [e.g., survive in the acidic environment of the stomach and to grow in the intestinal environment]), along with preservatives, stabilizers, binders, compaction agents, lubricants, dispersion enhancers, disintegration agents, antioxidants, flavoring agents, sweeteners, and coloring agents.

In one embodiment of any of the above methods involving administration of a probiotic composition, the probiotic composition is administered conjointly with a prebiotic which stimulates growth and/or activity of bacteria contained in the probiotic composition. Non-limiting examples of useful prebiotics include, e.g., galactose, β-N-Acetyl-α-glucosamine, pyroglutamtamic acid, arginine, serine, glycine, fructooligosaccharides (FOS), galactooligosaccharides (GOS), human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, arabinose, maltose, lactose, sucrose, cellobiose, amino acids, alcohols, resistant starch (RS), electrolytes and any combinations thereof. In some embodiments, the electrolytes can modulate or balance the pH. In one specific embodiment, the probiotic and prebiotic are administered in one composition, or simultaneously as two separate compositions, or sequentially.

In one embodiment of any of the above methods, the method further comprises monitoring the subject's microbiota after the administration of the bacterial inoculum by: (a) determining a relative abundance of one or more bacterial taxa in a GI microbiota sample obtained from the subject (e.g., isolated from feces, intestines, etc.), and (b) comparing the relative abundance(s) determined in step (a) to (i) a predetermined standard value or (ii) to the abundance(s) of the same taxa in a control subject (e.g., a healthy subject) or (iii) to the average value of abundances of the same taxa in several control subjects. Non-limiting examples of the methods which can be used for determining the relative abundance of the bacterial taxa include, e.g., quantitative polymerase chain reaction (qPCR), sequencing of bacterial 16S rRNA, shotgun metagenome sequencing, bacterial genotype pattern based fingerprinting (DNA fingerprinting), and metabolomics. In one specific embodiment, the method involves determining a relative abundance of the bacteria from the taxa listed in Table 1.

In certain embodiments of any of the above methods of the invention, the method further comprises administering a probiotic and/or a prebiotic composition, wherein the composition(s) stimulate growth and/or activity of one or more strains of bacteria in the gastrointestinal microbiota, or a compound or composition, wherein the compound or composition inhibits growth and/or activity of one or more strains of bacteria in the gastrointestinal microbiota.

Probiotic and/or prebiotic compositions useful in the methods and/or compositions of the invention include those described in the International Application PCT/US18/17052, which is incorporated herein by reference in its entirety for all purposes.

Compound or compositions that inhibit growth and/or activity of one or more strains of bacteria in the pancreatic and/or gastrointestinal microbiota include those described in the International Application PCT/US18/17052, which is incorporated herein by reference in its entirety for all purposes.

In certain embodiments, the compound or composition is administered to the subject by a route selected from the group consisting of oral, rectal (e.g., by enema), mucosal, topical, sublingual, intravenous and via naso/oro-gastric gavage.

In certain embodiments, the method described herein encompasses administering two or more compounds or compositions that inhibit growth and/or activity of one or more strains of fungi and/or bacteria to the same subject. Such compounds or compositions can be administered simultaneously or sequentially.

In one embodiment of any of the above methods of the invention, the compound or composition that inhibits growth and/or activity of one or more strains of fungi and/or bacteria is administered in a therapeutically effective amount. The dosages of the compound or composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to reduce or eradicate colonization.

In one embodiment of any of the above methods of the invention, the compound or composition is delivered to the subject in a form of a composition which comprises a carrier and/or excipient.

In one aspect, the invention provides a method for treating (including preventing) SE or NORSE in a subject in need thereof, said method comprising a compound or composition, wherein the compound or composition inhibits growth and/or activity of one or more strains of bacteria. In certain embodiment, the growth is inhibited to the extent that the bacterial strains are removed from the microbiota (i.e., reduced or ablated).

In one embodiment of any of the above methods of the invention, the compound or composition that inhibits growth and/or activity of one or more strains of bacteria is administered in a therapeutically effective amount. The dosages of the compound or composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to reduce or eradicate colonization.

In some embodiments, the compound or composition that inhibits growth and/or activity of one or more strains of bacteria according to any of the above methods can be natural products that inhibit microbial growth. In certain embodiments, the compound or composition that inhibits growth and/or activity of one or more strains of bacteria according to any of the above methods can be bacteria that is conditionally lethal engineered bacteria (e.g., H. Pylori, E. coli, etc. . . . ). In certain embodiments, the compound or composition that inhibits growth and/or activity of one or more strains of bacteria according to any of the above methods can be genetically engineered commensals strains of microorganisms. In some embodiments, suppressing growth or activity of at least one bacterial species in the microbiota according to any of the above methods involving such suppression can be achieved, e.g., by administering an antibiotic. In one specific embodiment, the antibiotic is administered in a therapeutic dose. In another specific embodiment, the antibiotic is administered in a sub-therapeutic dose.

Non-limiting examples of antibiotics useful in the methods of the invention include beta-lactams (e.g., Penicillin VK, Penicillin G, Amoxicillin trihydrate), nitroimidazoles, macrolides (e.g., Tylosin tartrate, Erythromycin, Azithromycin, and Clarithromycin), tetracyclines, glycopeptides (e.g., Vancomycin), and fluoroquinolones. In one specific embodiment, the method comprises administering Penicillin VK or Penicillin G at 1 mg/kg body weight per day for at least four weeks of life. In another specific embodiment, the method comprises administering Amoxicillin trihydrate at 25 mg/kg body weight per day for 1 to 3 treatments each lasting 3 to 5 days. In yet another specific embodiment, the method comprises administering Tylosin tartrate at 50 mg/kg body weight per day for 1 to 3 treatments each lasting 3 to 5 days.

### Pharmaceutical Compositions, Formulations, and Combination Treatments

In one aspect, the present disclosure provides a pharmaceutical composition comprising a compound or composition which can inhibit growth and/or activity of one or more strains of from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In certain embodiments, the composition comprises at least 1, 2, 3, 4, 5, 6, 7, 8, 9, 10, 20, 50 or more strains from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In some embodiments, only nonpathogenic species within the taxa qualify for use in the probiotics or methods herein.

In one aspect, the present disclosure provides a pharmaceutical composition comprising a compound or composition which can inhibit growth and/or activity of one or more strains of from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In certain embodiments, the composition comprises at least 1, 2, 3, 4, 5, 6, 7, 8, 9, 10, 20, 50 or more strains from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In some embodiments, only nonpathogenic species within the taxa qualify for use in the probiotics or methods herein.

In one aspect, the present disclosure provides a pharmaceutical composition comprising a compound or composition which can stimulate the growth and/or activity of one or more strains of from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In certain embodiments, the composition comprises at least 1, 2, 3, 4, 5, 6, 7, 8, 9, 10, 20, 50 or more strains from the taxa listed in FIG. 2, 4, 6, or 10 or Table 1 or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA. In some embodiments, only nonpathogenic species within the taxa qualify for use in the probiotics or methods herein.

In one aspect, the present disclosure provides a pharmaceutical composition comprising a compound or composition which can stimulate the growth and/or activity of one or more strains of from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In certain embodiments, the composition comprises at least 1, 2, 3, 4, 5, 6, 7, 8, 9, 10, 20, 50 or more strains from the taxa listed in FIG. 2, 4, 6 or 9 or Table 1 or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA. In some embodiments, only nonpathogenic species within the taxa qualify for use in the probiotics or methods herein.

In one aspect, the present disclosure provides a pharmaceutical composition comprising (i) a compound or composition which can inhibit growth and/or activity of one or more strains of fungi from the class Saccharomycetales, and optionally (ii) a therapeutic agent. In certain embodiments, the present disclosure provides a pharmaceutical composition comprising (i) a compound or composition which can inhibit growth and/or activity of one or more strains of fungi from the genus Nakaseomyces and Marasmius in the gastrointestinal microbiota of a subject, and optionally (ii) a therapeutic agent. In certain embodiments, the compound or composition can inhibit growth and/or activity of one or more strains of fungi from the species Nakaseomyces glabratus and Marasmius oreades.

In one aspect, the present disclosure provides a pharmaceutical dosage form comprising (i) a compound or composition which can inhibit growth and/or activity of one or more strains of fungi from the class Saccharomycetales in the gastrointestinal microbiota of a subject, and optionally (ii) therapeutic agent. In certain embodiments, the present disclosure provides a pharmaceutical dosage form comprising (i) a compound or composition which can inhibit growth and/or activity of one or more strains of fungi from the genus Nakaseomyces and Marasmius in the gastrointestinal microbiota of a subject, and optionally (ii) therapeutic agent. In certain embodiments, the compound or composition can inhibit growth and/or activity of one or more strains of fungi from the species Nakaseomyces glabratus and Marasmius oreades.

In certain embodiments of the pharmaceutical composition or pharmaceutical dosage form, the compound or composition that inhibits growth and/or activity of one or more strains of fungi from the genus Nakaseomyces and Marasmius in the gastrointestinal microbiota comprises an antifungal compound or a natural product that inhibits fungal growth. The antifungal compound may be selected from an echocandin compound, a triazole compound, and a polyene compound, and any combinations thereof. In certain embodiments, the antifungal compound is amphotericin B or fluconazole.

In certain embodiments, the pharmaceutical composition or pharmaceutical dosage form further comprises a second compound or composition that stimulates growth and/or activity of one or more strains of fungi from the genera Nakaseomyces and Marasmius in the gastrointestinal microbiota of the subject. In certain embodiments, the composition comprises one or more strains of fungi from the genera Nakaseomyces and Marasmius.

In one aspect, the present invention provides a method for enhancing efficacy of a treatment for SE or NORSE subject in need thereof, said method comprising (i) administering said treatment to the subject and further (ii) administering to the subject an effective amount of a compound or composition, wherein the compound or composition inhibits growth and/or activity of one or more strains of fungi from the genus Nakaseomyces and Marasmius in the gastrointestinal microbiota of the subject.

In one aspect, the present disclosure provides a pharmaceutical composition comprising (i) a compound or composition which can stimulate the growth and/or activity of one or more strains of fungi from the genus Trichoderma and Fusarium in the gastrointestinal microbiota of a subject, and optionally (ii) a therapeutic agent. In certain embodiments, the compound or composition can simulate the growth and/or activity of one or more strains of fungi from the species Trichoderma breve and Fusarium verticillioides.

In one aspect, the present disclosure provides a pharmaceutical dosage form comprising (i) a compound or composition which can stimulate the growth and/or activity of one or more strains of fungi from the genus Trichoderma and Fusarium in the gastrointestinal microbiota of a subject, and optionally (ii) therapeutic agent. In certain embodiments, the compound or composition can stimulate the growth and/or activity of one or more strains of fungi from the species Trichoderma breve and Fusarium verticillioides.

In certain embodiments of the pharmaceutical composition or pharmaceutical dosage form, the compound or composition that stimulate the growth and/or activity of one or more strains of fungi from the genus Trichoderma and Fusarium in the gastrointestinal microbiota comprises an compound or a natural product that stimulates fungal growth.

In certain embodiments, the pharmaceutical composition or pharmaceutical dosage form further comprises a second compound or composition that stimulates the growth and/or activity of one or more strains of fungi from the genera Trichoderma and Fusarium in the gastrointestinal microbiota of the subject. In certain embodiments, the composition comprises one or more strains of fungi from the genera Trichoderma and Fusarium or species Trichoderma breve and Fusarium verticillioides.

In one aspect, the present invention provides a method for enhancing efficacy of a treatment for SE or NORSE subject in need thereof, said method comprising (i) administering said treatment to the subject and further (ii) administering to the subject an effective amount of a compound or composition, wherein the compound or composition stimulates the growth and/or activity of one or more strains of fungi from the genus Trichoderma and Fusarium or species Trichoderma breve and Fusarium verticillioides in the gastrointestinal microbiota of the subject.

In one aspect, the present disclosure provides a pharmaceutical composition comprising (i) a compound or composition which can inhibit growth and/or activity of one or more strains of bacteria from the genus Enterococcus, Enterococcus, Escherichia, Sellimonas, Akkermansia, Lacticaseibacillus, Streptococcus, and Streptococcus in the gastrointestinal microbiota of a subject, and optionally (ii) a therapeutic agent. In certain embodiments, the compound or composition can inhibit growth and/or activity of one or more strains of bacteria from the species Enterococcus faecalis, Enterococcus faecium, Enterocloster bolteae, Escherichia coli, Sellimonas intestinalis, Akkermansia muciniphila, Fusicatenibacter saccharivorans, Lacticaseibacillus rhamnosus, Lacticaseibacillus paracasei, Streptococcus anginosus, and Roseburia hominis.

In one aspect, the present disclosure provides a pharmaceutical dosage form comprising (i) a compound or composition which can inhibit growth and/or activity of one or more strains of bacteria from the genus Enterococcus, Enterococcus, Escherichia, Sellimonas, Akkermansia, Lacticaseibacillus, Streptococcus, and Streptococcus in the gastrointestinal microbiota of a subject, and optionally (ii) therapeutic agent. In certain embodiments, the compound or composition can inhibit growth and/or activity of one or more strains of bacteria from the species Enterococcus faecalis, Enterococcus faecium, Enterocloster bolteae, Escherichia coli, Sellimonas intestinalis, Akkermansia muciniphila, Fusicatenibacter saccharivorans, Lacticaseibacillus rhamnosus, Lacticaseibacillus paracasei, Streptococcus anginosus, and Roseburia hominis.

In certain embodiments of the pharmaceutical composition or pharmaceutical dosage form, the compound or composition that inhibits growth and/or activity of one or more strains of bacteria from the genus Enterococcus, Enterococcus, Escherichia, Sellimonas, Akkermansia, Lacticaseibacillus, Streptococcus, and Streptococcus in the gastrointestinal microbiota comprises an antibiotic compound or a natural product that inhibits bacterial growth.

In certain embodiments, the pharmaceutical composition or pharmaceutical dosage form further comprises a second compound or composition that stimulates growth and/or activity of one or more strains of bacteria from the genera Enterococcus, Enterococcus, Escherichia, Sellimonas, Akkermansia, Lacticaseibacillus, Streptococcus, and Streptococcus in the gastrointestinal microbiota of the subject. In certain embodiments, the composition comprises one or more strains of bacteria from the genera Enterococcus, Enterococcus, Escherichia, Sellimonas, Akkermansia, Lacticaseibacillus, Streptococcus, and Streptococcus.

In one aspect, the present invention provides a method for enhancing efficacy of a treatment for SE or NORSE subject in need thereof, said method comprising (i) administering said treatment to the subject and further (ii) administering to the subject an effective amount of a compound or composition, wherein the compound or composition inhibits growth and/or activity of one or more strains of bacteria from the genus Enterococcus, Enterococcus, Escherichia, Sellimonas, Akkermansia, Lacticaseibacillus, Streptococcus, and Streptococcus in the gastrointestinal microbiota of the subject.

In one aspect, the present disclosure provides a pharmaceutical composition comprising (i) a compound or composition which can stimulate the growth and/or activity of one or more strains of bacteria from the genus Clostridium, Bacteroides, Fusicatenibacter, and Eubacterium in the gastrointestinal microbiota of a subject, and optionally (ii) a therapeutic agent. In certain embodiments, the compound or composition can simulate the growth and/or activity of one or more strains of bacteria from the species Clostridium leptum, Bacteroides stercoris, Fusicatenibacter saccharivorans and Eubacterium rectale.

In one aspect, the present disclosure provides a pharmaceutical dosage form comprising (i) a compound or composition which can stimulate the growth and/or activity of one or more strains of bacteria from the genus Clostridium, Bacteroides, Fusicatenibacter, and Eubacterium in the gastrointestinal microbiota of a subject, and optionally (ii) therapeutic agent. In certain embodiments, the compound or composition can stimulate the growth and/or activity of one or more strains of bacteria from the species Clostridium leptum, Bacteroides stercoris, Fusicatenibacter saccharivorans and Eubacterium rectale.

In certain embodiments of the pharmaceutical composition or pharmaceutical dosage form, the compound or composition that stimulate the growth and/or activity of one or more strains of bacteria from the genus Clostridium, Bacteroides, Fusicatenibacter, and Eubacterium in the gastrointestinal microbiota comprises a compound or a natural product that stimulates bacterial growth.

In certain embodiments, the pharmaceutical composition or pharmaceutical dosage form further comprises a second compound or composition that stimulates the growth and/or activity of one or more strains of bacteria from the genera Clostridium, Bacteroides, Fusicatenibacter, and Eubacterium in the gastrointestinal microbiota of the subject. In certain embodiments, the composition comprises one or more strains of bacteria from the genera Clostridium, Bacteroides, Fusicatenibacter, and Eubacterium or species Clostridium leptum, Bacteroides stercoris, Fusicatenibacter saccharivorans and Eubacterium rectale.

In one aspect, the present invention provides a method for enhancing efficacy of a treatment for SE or NORSE subject in need thereof, said method comprising (i) administering said treatment to the subject and further (ii) administering to the subject an effective amount of a compound or composition, wherein the compound or composition stimulates the growth and/or activity of one or more strains of bacteria from the genus Clostridium, Bacteroides, Fusicatenibacter, and Eubacterium or species Clostridium leptum, Bacteroides stercoris, Fusicatenibacter saccharivorans and Eubacterium rectale in the gastrointestinal microbiota of the subject.

In certain embodiments, the compound or composition that inhibit growth and/or activity of one or more strains of fungi and/or bacteria and the therapeutic methods/agents can be administered in one composition. In certain embodiments, the compound or composition that inhibit growth and/or activity of one or more strains of fungi and/or bacteria and the therapeutic methods/agents can be administered in different compositions.

In certain embodiments, the pharmaceutical composition or pharmaceutical dosage form further comprises one or more of the following (i) a probiotic and/or a prebiotic composition that stimulates growth and/or activity of one or more strains of fungi and/or bacteria from one or more genera selected from FIG. 2, 4, 6, 9, or 10 or Table 1 in the GI microbiota of the subject; (ii) a probiotic and/or a prebiotic composition that stimulates growth and/or activity of one or more strains of fungi and/or bacteria from one or more species selected from FIG. 2, 4, 6, 9, or 10 or Table 1 in the gastrointestinal (GI) microbiota of the subject; (iii) a probiotic composition comprising one or more strains of fungi and/or bacteria from one or more genera selected from FIG. 2, 4, 6, 9, or 10 or Table 1; (iv) a probiotic composition comprising one or more strains of fungi and/or bacteria from one or more species selected from FIG. 2, 4, 6, 9, or 10 or Table 1; (v) a compound or composition which inhibits growth and/or activity of one or more strains of fungi and/or bacteria from one or more genera selected from FIG. 2, 4, 6, 9, or 10 or Table 1 in the gastrointestinal (GI) microbiota of the subject; or (vi) a compound or composition which inhibits growth and/or activity of one or more strains of fungi and/or bacteria from one or more species selected from FIG. 2, 4, 6, 9, or 10 or Table 1 in the gastrointestinal (GI) microbiota of the subject.

Oral delivery may include the use of nanoparticles that can be targeted, e.g., to the GI tract of the subject, such as those described in Yun et al., Adv Drug Deliv Rev. 2013, 65(6):822-832 (e.g., mucoadhesive nanoparticles, negatively charged carboxylate- or sulfate-modified particles, etc.). Non-limiting examples of other methods of targeting delivery of compositions to the GI tract are discussed in U.S. Pat. Appl. Pub. No. 2013/0149339 and references cited therein (e.g., pH sensitive compositions [such as, e.g., enteric polymers which release their contents when the pH becomes alkaline after the enteric polymers pass through the stomach], compositions for delaying the release [e.g., compositions which use hydrogel as a shell or a material which coats the active substance with, e.g., in vivo degradable polymers, gradually hydrolyzable polymers, gradually water-soluble polymers, and/or enzyme degradable polymers], bioadhesive compositions which specifically adhere to the colonic mucosal membrane, compositions into which a protease inhibitor is incorporated, a carrier system being specifically decomposed by an enzyme present in the colon).

For oral administration, the active ingredient(s) can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

It is also contemplated that when used to treat SE or NORSE, the compositions and methods of the present invention can be utilized with other therapeutic methods/agents suitable for the same or similar disorders. Such other therapeutic methods/agents can be co-administered (simultaneously or sequentially) to generate additive or synergistic effects. Suitable therapeutically effective dosages for each agent may be lowered due to the additive action or synergy.

In one embodiment of any of the above methods of the invention, the method further comprises administering to the subject one or more additional compounds selected from immuno-suppressives, biologicals, probiotics, prebiotics, and cytokines (e.g., IFN or IL-22), and any combinations thereof.

As a non-limiting example, the invention can be combined with other therapies that block inflammation (e.g., via blockage of IL1, INFα/β, IL6, TNF, IL23, etc.).

In certain embodiments, a conditional lethal bacterial strain can be utilized as the inoculant or to deliver a recombinant construct. Such a conditional lethal bacteria survives for a limited time typically when provided certain nutritional supplements. It is contemplated that such a supplement could be a liquid, formulated to contain the nutritional component necessary to keep the bacteria alive. It is further contemplated that a patient/subject would drink such a supplement in intervals to keep the bacteria alive. Once the supplement is depleted, the conditional lethal bacteria die. Methods relating to conditional lethal strains of H. pylori are described in U.S. Pat. No. 6,570,004.

Spores used in the compositions of the invention can be isolated, for example, by solvent treatments (e.g., using partially miscible, fully miscible or an immiscible solvent), chromatographic treatments (e.g., using hydrophobic interaction chromatography (HIC) or an affinity chromatography), mechanical treatments (e.g., blending, mixing, shaking, vortexing, impact pulverization, and sonication), filtration treatments, thermal treatments (e.g., 30 seconds in a 100° C. environment followed by 10 minutes in a 50° C.), irradiation treatments (e.g., with ionizing radiation, typically gamma irradiation, ultraviolet irradiation or electron beam irradiation provided at an energy level sufficient to kill pathogenic materials while not substantially damaging the desired spore populations), centrifugation and density separation treatments (e.g., using density or mobility gradients or cushions (e.g., step cushions), such as, e.g., CsCl, Percoll, Ficoll, Nycodenz, Histodenz or sucrose gradients). It is generally desirable to retain the spore populations under non-germinating and non-growth promoting conditions and media, in order to minimize the growth of pathogenic bacteria present in the spore populations and to minimize the germination of spores into vegetative bacterial cells.

The compositions of the invention can comprise a carrier and/or excipient. While it is possible to use a bacterial inoculant or compound of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate, infant formula, or other agents which allow bacteria to survive and grow [e.g., survive in the acidic environment of the stomach and to grow in the intestinal environment]), preservatives, stabilizers, binders, compaction agents, lubricants, dispersion enhancers, disintegration agents, antioxidants, flavoring agents, sweeteners, and coloring agents. Additional specific examples of suitable carriers and/or excipients include, e.g., vegetable cellulose, vegetable stearic acid, vegetable magnesium stearate, and/or silica. Those of relevant skill in the art are well able to prepare suitable solutions.

In some embodiments, the methods described herein include administering to the subject determined to SE or NORSE a treatment for normalizing an intestinal barrier with increased permeability and/or normalizing the composition of the intestinal microbial community. In some embodiments, normalizing the composition of the intestinal microbial community comprises controlling growth and/or activity of a fungal and/or bacterial strain, such as for example and not limitation, by administration of an antifungal composition and/or antibiotic and/or probiotic and/or prebiotic and/or bacteriophage which inhibits growth or activity of the fungal and/or bacterial strain, as further described herein.

In some embodiments, the compound or composition interferes with fungal metabolic and/or biosynthetic pathways to affect proliferation or depth of the strains. In some embodiments, the composition is administered to the subject in an effective amount sufficient to interfere with fungal metabolic and/or biosynthetic pathways.

In some embodiments, the compound or composition interferes with bacterial metabolic and/or biosynthetic pathways to affect proliferation or depth of the strains. In some embodiments, the composition is administered to the subject in an effective amount sufficient to interfere with bacterial metabolic and/or biosynthetic pathways.

In certain embodiments, suppressing growth or activity of at least one bacterial species in the microbiota according to any of the above methods involving such suppression can be achieved, e.g., by administering a bacteriophage, a modified bacteriophage, or a compound which integrates the selective binding function or biologic activities of bacteriophage. In one specific embodiment, the bacteriophage is administered in a therapeutic dose. In another specific embodiment, the bacteriophage is administered in a sub-therapeutic dose. The bacteriophage is capable of interacting with the disclosed species in such a way as to suppress its growth or activity. This could be by suppression of lipoglycan production by the bacterium.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Methods

### Study Design and Participants

In a longitudinal cohort study, subjects with NORSE, SE, and chronic epilepsy were recruited. Subjects with NORSE and SE underwent longitudinal serial biospecimen collection over the course of their illness, while subjects with chronic epilepsy controls underwent a single biospecimen collection.

Subjects with NORSE were recruited through the NORSE Consortium, a large-scale effort coordinated at Yale University, involving 33 academic centers across North and South America and Europe since 2016. Subjects met consensus definition criteria for NORSE, i.e., SE refractory to first and second-line therapy, no clear acute or active structural, toxic, or metabolic cause found despite extensive work-up, and age ≥2 years old. Exclusion criteria included an active epilepsy diagnosis or other relevant neurological disorder, and SE with fully retained consciousness (Hirsch et al., 2018). Investigators at Yale University School of Medicine, the leader of the NORSE Consortium, adjudicate all NORSE cases and ensure diagnostic criteria meet consensus definitions. Gut microbiome specimen collection was completed in 15 subjects across 10 sites in North America and Europe. Each site obtained ethics approval.

SE control subjects were recruited at New York University (NYU) Langone Hospital, through regular review of the continuous EEG service. Subjects older than 2 years old with SE of known cause, including structural, metabolic, infectious, toxic, and genetic etiologies were included.

Chronic epilepsy controls were identified at the NYU Comprehensive Epilepsy Center from December 2022 to June 2023 through review of the epilepsy monitoring unit planned admission list. Adult patients with an established diagnosis of epilepsy and a planned elective admission to the epilepsy monitoring unit were recruited. Those on the ketogenic diet, experiencing frequent (more than weekly) seizures, with a body mass index (BMI) >40 kg/m a diagnosis of irritable bowel syndrome, history of colon cancer, colonoscopy and antibiotic treatment ≤30 days prior to data collection, and major surgery ≤3 months prior to data collection were excluded in order to avoid additional factors affecting microbiome composition.

TABLE 2
Demographics and comorbidities of subjects with status epile-ticus (SE)-
including SE of known cause and new onset refractory
status epilepticus (NORSE)-versus chronic epilepsy controls

| | Status | Chronic | |
|---|---|---|---|
| | Epilepticus | Epilepsy | |
| | (n = 32) | (n = 12) | p |
| Age, Mdn | 62 (4-89, 45) | 37 (18-58, 23.25) | .047* |
| (min-max, IQR) | | | |
| Sex, F (%) | 18 (58.1) | 7 (58.3) | 1.000 |
| Race | | | .515 |
| American Indian/ | 1 (3.1) | 0 | |
| Alaskan Native (%) | | | |
| Asian (%) | 1 (3.1) | 1 (8.3) | |
| Black/African | 3 (9.4) | 0 | |
| American (%) | | | |
| Native Hawaiian/ | 0 | 1 (8.3) | |
| Pacific Islander | | | |
| White (%) | 24 (75.0) | 10 (83.3) | |
| Other (%) | 1 (3.1) | 0 | |
| Unknown (%) | 2 (6.3) | 0 | |
| Ethnicity, Hispanic/ | 3 (9.7) | 2 (16.7) | .608 |
| Latino (%) | | | |
| History of kidney/liver | 11 (34.4) | 2 (16.7) | .459 |
| dysfunction (%) | | | |
| History of proton pump | 11 (34.4) | 1 (8.3) | .132 |
| inhibitor use (%) | | | |
| History of autoimmune | 7 (21.8) | 1 (8.3) | .287 |
| disease (%) | | | |
| History of tobacco/ | 9 (28.1) | 1 (8.3) | .241 |
| nicotine use (%) | | | |
| History of alcohol/ | 4 (12.5) | 1(8.3) | 1.000 |
| drug abuse (%) | | | |
| History of cancer (%) | 4 (12.5) | 0 | .562 |
| History of colon | 1 (3.1) | 0 | 1.000 |
| cancer (%) | | | |
| History of HIV/AIDS | 2 (6.3) | 0 | 1.000 |
| diagnosis (%) | | | |
| Number of anti-seizure | 5 (1-14, 2.75) | 2 (1-4, 2) | <.001 |
| medications used during | | | |
| study participation, | | | |
| Mdn (min-max, IQR) | | | |

### Procedures

Human fecal and blood samples were collected from NORSE and SE subjects at a prespecified biospecimen collection schedule as follows: day of enrollment (as early as possible in hospital course), and days 2, 3-4, 8, 14, 28, and 56 post-enrollment, if subjects were still hospitalized. The majority of subjects did not have a fecal sample collected at each of these time points, as expected given frequent GI dysmotility in critically ill subjects. Post-hoc, samples were categorized into two: pre-SE resolution/within 3 days of resolution (T1) and >4 days post SE resolution (T2). The date of last seizure was used to determine end of SE for control subjects, and date subject was taken off continuous intravenous antiseizure treatment was considered end of SE for NORSE subjects, given their protracted treatment course and frequent prolonged anesthetic use. T1 samples included samples collected pre-SE resolution and up to 3 days after SE resolution. T2 samples included samples collected at least 4 days post SE resolution. If multiple samples existed for a given subject, the earliest T1 sample and latest T2 sample was selected for analysis. Approximately 50 g or 3-5 cm of fecal matter were collected using the OMR-200 OMNIgene-GUT kit.

### Fecal Sample Whole-Community DNA Extraction

Status and epilepsy control subject fecal samples were collected in OMNIgene-GUT OM-200 kit by (DNA Genotek, Ottawa, Ontario, Canada) and were shipped at room temperature to the laboratory of Dr. Deepak Saxena within 24-48 hours of collection and stored at −80° C. until further processing. Aliquots of homogenized sample were subjected to DNA extraction using a Qiagen QIAamp PowerFecal Pro DNA kit according to the manufacturer's instructions. Extracted DNA was quantified using Quant-iT PicoGreen dsDNA Assay Kit (Invitrogen, Waltham, MA, USA) and checked for purity by NanoDrop Spectrophotometer.

### DNA Library Construction and Shotgun Metagenomic Sequencing

DNA samples were normalized to a concentration of 1.6 ng/μL prior to performing library construction. Shotgun metagenomic DNA libraries were constructed with 24 ng of DNA per sample using Illumina DNA Prep containing the Illumina Purification Beads and IDT for Illumina DNA/RNA UD Indexes according to the manufacturer's instructions for samples <100 ng DNA, using 8 PCR cycles (Doc #1000000025416 v10, Illumina, San Diego, CA, USA). Libraries were pooled in equimolar amounts, a 1.0% PhiX Control (v3) spike-in was applied, and the pooled library was sequenced on an Illumina NextSeq 1000 platform with a 300-cycle (2×150) P2 reagent kit (V3). Negative controls were handled exactly as samples and consisted of water and buffer used in DNA extraction and dilutions. ZymoBIOMICS Fecal Reference with TruMatrix Technology (ZYMO Research, Irvine, CA, USA) was included as a sequencing quality control and to facilitate interstudy comparisons.

### Clinical Covariates

Chart review was completed to ascertain relevant premorbid characteristics to microbiome composition (history of kidney/liver dysfunction, proton pump inhibitor (PPI) use, autoimmune diseases, tobacco or alcohol use, cancer, HIV and cancer) in all subjects. For the NORSE and SE subjects, critical illness characteristics with a potential impact on microbiome were also collected, including anesthetic use, antibiotic use, sepsis, intubation, feeding, and immunotherapy.

### Outcome Ascertainment

Outcome was ascertained through chart review and caregiver telephone contact if there was no documented follow-up available by 3 months then 12 months. Disability was ascertained through modified Rankin scale administration (>4 considered disabled), and subjects and caregivers were questioned on the presence of epilepsy at follow-up.

### Statistical and Bioinformatic Analyses

### Metagenomics Analysis.

Paired end raw sequencing reads in FASTQ format were quality controlled using KneadData v0.10.0 pipeline (https://huttenhower.sph.harvard.edu/kneaddata/), including low-quality read trimming using Trimmomatic (Bolger et al., 2014), and decontamination of host DNA reads using bowtie2 (Langmead & Salzberg, 2012; Langmead et al., 2018) against the human genome reference GRCh37/hgl9. Decontaminated clean metagenomic reads were then classified using both MetaPhlAn 4.0.4 and Kraken 2.1.3 to profile the species level taxonomic community in each sample (Blanco-Miguez et al., 2023; Wood et al., 2019). Kraken2 species abundances were then re-estimated using Bracken (Lu et al., 2017). HUMAnN 3.7 was used to profile the abundance of UniRef90 gene families and microbial metabolic pathways in the samples (Beghini et al., 2021). The profiled functional features in reads per kilobase (RPK) units were transformed to relative abundance for later analysis.

Taxonomic profiles of Bacteria and Archaea were extracted from the MetaPhlAn4 outputs, and the eukaryotic(fungi) profiles were extracted from the Bracken corrected Kraken2 reports for further analysis. Taxonomic and functional profiles were imported into phyloseq objects in R to perform downstream analysis (McMurdie & Holmes, 2013). Alpha diversity metrics were calculated using microbiome package (version 1.20.0). A Wilcoxon rank-sum test was used to identify any significant differences in the observed number of species and Shannon indices across different comparative groups. Beta diversity analysis were performed on Bray-Curtis dissimilarity metrics and visualized by principal coordinate analysis (PCoA), and statistical difference was accessed by Permutational multivariate analysis of variance (PERMANOVA) using the Vegan r package (2.6-4). Differentially abundant taxa between groups were identified by Linear discriminant analysis effect size (LEfSe), and significantly enriched species were determined with LDA scores >3. Multivariate Associations by Linear models (MaAsLin2) was used to assess the differentially abundant gene families and MetaCyc pathways. An adjusted p value of <0.1 was considered significant for all MaAsLin2 tests performed in functional analysis. Unmapped and ungrouped functional features were not included in the analysis.

### Example 2. Characteristics of NORSE, SE, and Epilepsy Subjects

The low incidence of NORSE has hampered efforts to elucidate its mechanisms and improve outcomes. The NORSE Consortium was established in 2016 to collaboratively recruit a critical number of subjects with NORSE across 33 academic centers in North and South America and Europe. In this Consortium, NORSE and SE disease controls undergo longitudinal clinical assessments, along with serial biospecimens, including blood, cerebrospinal fluid (CSF), and stool collection.

In the current Example, fecal microbiome composition was examined from 15 subjects hospitalized with NORSE, 17 subjects hospitalized with SE of known cause subjected to similar critical illness conditions, and 12 chronic epilepsy controls without SE or critical illness. Fecal shotgun metagenomics were utilized, characterizing whole-community DNA, and described differences in taxonomic and functional diversity of prokaryotes and eukaryotes, and correlated microbiome with cytokines, relevant clinical covariates and neurologic outcomes.

The thirty-two Subjects with SE (including both NORSE, n=15, and SE of known cause, n=17) were older than subjects with chronic epilepsy (n=12) (Table 2). Subjects with NORSE were more frequently female (80% vs 35.3%, p=0.016) and were younger than those with SE of known cause (median 32, IQR 48, vs 70, IQR 17, p=0.004) but there were no significant differences in racial background between all groups. There were no differences in premorbid conditions between all SE and chronic epilepsy, but within the SE group, subjects with SE of known cause were more likely to have premorbid kidney or liver dysfunction and PPI use than subjects with NORSE (Table 3). Clinical histories for all NORSE and SE subjects are outlined in Table 4. NORSE was largely cryptogenic (12/15 subjects), with an infectious (West Nile Virus) and autoimmune (myelin oligodendrocyte glycoprotein antibody-associated disease and neuro-Behcet's) identified in a minority after extensive workup. SE of known cause (referred to as SE controls below) was of structural (n=9), toxic/metabolic (n=5), or infectious (n=3) cause (specific etiologies outlined in Table 4).

TABLE 3
Comorbidities, ICU characteristics and clinical outcomes of
NORSE and status epilepticus of known cause

| | | SE of | |
|---|---|---|---|
| | | known | |
| | NORSE | cause | p |
| Pre-Status Epilepticus Conditions | | | |
| Kidney/liver dysfunction | 1 (6.7%) | 10 (58.8%) | .003* |
| Proton pump inhibitor use | 1 (6.7%) | 10 (58.8%) | .003* |
| Autoimmune disease | 5 (33.3%) | 2 (11.8%) | .210 |
| Nicotine use | 4 (26.7%) | 5 (29.4%) | 1.000 |
| Alcohol/drug abuse | 2 (13.3%) | 2 (11.8%) | 1.000 |
| Cancer | 0 | 4 (23.5%) | .104 |
| Colon cancer | 0 | 1 (5.9%) | 1.000 |
| HIV/AIDS | 0 | 2 (11.8%) | .486 |
| ICU/Treatment Conditions | | | |
| Antibody Positive | 1 (6.7%) | 0 | .469 |
| Anesthesia use | 15 (100%) | 13 (76.5%) | .104 |
| Antibiotic use | 414 (93.3%) | 15 (88.2%) | 1.000 |
| Cell wall | 14 (93.3%) | 15 (88.2%) | 1.000 |
| Cell membrane | 0 | 2 (6.3%) | .486 |
| Protein synthesis | 2 (13.3%) | 5 (29.4%) | .403 |
| Nucleic acid synthesis | 1 (6.7%) | 5 (29.4%) | .178 |
| DNA/oxidative damage | 0 | 1 (5.9%) | 1.000 |
| Infection | 8 (53.3%) | 12 (70.6%) | .467 |
| Metabolic disturbance | 1 (8.3%) | 8 (50%) | .039* |
| Liver dysfunction | 7 (50.0%) | 2 (11.8%) | .044* |
| Kidney dysfunction | 0 | 3 (17.7%) | .232 |
| Intubation | 12 (80.0%) | 12 (70.6%) | .691 |
| Tube Feeding | 11 (84.6%) | 12 (70.6%) | .427 |
| Ketogenic Diet | 5 (33.3%) | 0 | .015* |
| Immunotherapy | 14 (93.3%) | 3 (17.7%) | <.001* |
| Length of SE (days), | 18 (2-90, 22) | 3 (1-17, 4) | .003* |
| Mdn (min-max, IQR) | | | |
| Number of ASMs administered, | 7 (4-14, 4) | 4 (1-6, 2.5) | <.001* |
| Mdn (min-max, IQR) | | | |
| Outcomes | | | |
| Mortality | | | |
| At time of retrospective | 4 (26.7) | 10 (58.8) | .087 |
| data collection (%) | | | |
| Within 3 months of status | 3 (20) | 4 (23.5) | 1.00 |
| epilepticus (%) | | | |
| Disability 12 months post status | 11 (73.3) | 11 (64.7) | .750 |
| epilepticus (%)^{a} | | | |
| Epilepsy 12 months post status | 7 (46.7) | 2 (11.8) | .015* |
| epilepticus (%) | | | |

^{a}Modified Rankin Scale ≥4
ASMs—antiseizure medications

As expected, subjects with NORSE experienced longer duration of SE, received more antiseizure medications (ASMs), and suffered from epilepsy more frequently at follow-up than their SE of known cause (control) counterparts (Table 3). NORSE and control SE subjects differed in their critical illness course: control SE subjects were more likely to experience metabolic disturbance, while NORSE subjects were more likely to experience liver dysfunction and be in SE for a longer duration and were more likely to be prescribed the ketogenic diet, immunotherapy, and a greater number of antiseizure medications (Table 3). Antibiotics were administered to the majority of the NORSE and SE control cohorts. Antibiotic mechanism was no different between NORSE and control SE subjects (Table 3), but penicillins were more often administered to subjects with NORSE (Table 5). All NORSE and SE subjects reported had outcome data available.

TABLE 4
Clinical summary of etiology of status
epilepticus among NORSE and SE controls

| | | | Length | | |
|---|---|---|---|---|---|
| | | | of | | |
| Subject | NORSE/ | | Status | Status | Clinical |
| ID | Control | Age | (Days) | Etiology | Summary |
| 1 | NORSE | 62 | 6 | Cryptogenic | Comorbid acute |
| (Yale0011) | | | | | renal failure, |
| | | | | | hypercalcemia, |
| | | | | | alcohol |
| | | | | | intoxication |
| 2 | NORSE | 45 | 12 | Cryptogenic | |
| (Yale0012) | | | | | |
| 3 | NORSE | 35 | 10 | Cryptogenic | |
| (MSSM005) | | | | | |
| 4 | NORSE | 77 | 12 | Infectious | West Nile Virus |
| (MSSM006) | | | | | |
| 5 | NORSE | 21 | 23 | Cryptogenic | |
| (NCA001) | | | | | |
| 6 | NORSE | 32 | 18 | Cryptogenic | |
| (OHSU001) | | | | | |
| 7 | NORSE | 62 | 32 | Cryptogenic | |
| (PHMJ001) | | | | | |
| 8 | NORSE | 12 | 31 | Cryptogenic | |
| (SCH002) | | | | | |
| 9 | NORSE | 8 | 17 | Autoimmune | MOGAD |
| (SCH003) | | | | | |
| 10 | NORSE | 74 | 5 | Cryptogenic | |
| (UCGNI002) | | | | | |
| 11 | NORSE | 14 | 45 | Cryptogenic | |
| (UCSF001) | | | | | |
| 12 | NORSE | 27 | 38 | Cryptogenic | |
| (UFL001) | | | | | |
| 13 | NORSE | 87 | 23 | Cryptogenic | |
| (YALE0013) | | | | | |
| 14 | NORSE | 4 | 90 | Cryptogenic | |
| (Yale0014) | | | | | |
| 15 | NORSE | 28 | 2 | Autoimmune | Behcet's Disease |
| (NORSE016) | | | | | |
| 16 | Control | 64 | 17 | Toxic/ | HIV |
| (MSSM004) | | | | Metabolic | hemodialysis, end |
| | | | | | stage renal failure |
| 17 | Control | 59 | 2 | Toxic/ | Seizures in the |
| (001) | | | | Metabolic | setting of drug |
| | | | | | intoxication. |
| 18 | Control | 65 | 3 | Structural | Cardioembolic |
| (002) | | | | | stroke |
| 19 | Control | 75 | 3 | Structural | Subdural |
| (003) | | | | | hematoma post |
| | | | | | shunt placement |
| 20 | Control | 73 | 8 | Infectious | Pneumonia in |
| (004) | | | | | setting of chronic |
| | | | | | neurodegenerative |
| | | | | | disorder |
| 21 | Control | 70 | 3 | Structural | Intraventricular |
| (005) | | | | | hemorrhage |
| 22 | Control | 83 | 2 | Structural | Acute subdural |
| (006) | | | | | hematoma |
| 23 | Control | 71 | 1 | Structural | Multifocal |
| (007) | | | | | intracranial |
| | | | | | atherosclerosis, |
| | | | | | history of prior |
| | | | | | stokes and |
| | | | | | meningitis, |
| | | | | | infection with |
| | | | | | Staphyloccocus |
| 24 | Control | 74 | 7 | Toxic/ | Complication |
| (008) | | | | Metabolic | from lung |
| | | | | | transplant |
| 25 | Control | 89 | 6 | Structural | Subdural |
| (010) | | | | | hematoma |
| 26 | Control | 38 | 2 | Infectious | Urinary tract |
| (011) | | | | | infection and |
| | | | | | aspiration |
| | | | | | pneumonia |
| 27 | Control | 75 | 1 | Infectious | Gallbladder |
| (012) | | | | | abscess, sepsis |
| 28 | Control | 13 | 6 | Structural | Optic tract glioma |
| (013) | | | | | with extension |
| | | | | | into brainstem |
| 29 | Control | 52 | 1 | Toxic/ | Diabetic |
| (014) | | | | Metabolic | ketoacidosis |
| 30 | Control | 70 | 2 | Structural | Hemispheric |
| (015) | | | | | cortical |
| | | | | | cavernoma |
| 31 | Control | 80 | 2 | Structural | Structural brain |
| (017) | | | | | disease in setting |
| | | | | | of active |
| | | | | | infections |
| 32 | Control | 58 | 5 | Toxic/ | Hyperglycemia |
| (018) | | | | Metabolic | |

TABLE 5
Antibiotic use during hospitalization of NORSE and SE controls.

| | | SE of | |
|---|---|---|---|
| | | known | |
| | NORSE | cause | p |
| Antibiotics | 14 (93.3%) | 15 (88.2%) | 1.000 |
| Class | | | |
| Penicillins | 4 (26.7%) | 0 | 0.038* |
| Macrolides | 1 (6.7%) | 1 (5.9%) | 1.000 |
| Cephalosporins | 12 (80.0%) | 12 (70.6%) | 0.691 |
| Fluoroquionolones | 0 | 3 (9.4%) | 0.229 |
| Aminopenecillins with Beta | 6 (40%) | 10 (58.8%) | 0.480 |
| Lactamase Inhibitors | | | |
| Tetracyclines | 0 | 2 (6.3%) | 0.486 |
| Carbapenems | 2 (13.3%) | 6 (35.3%) | 0.229 |
| Aminoglycosides | 1 (6.7%) | 3 (9.4%) | 0.603 |
| Lincosamides | 0 | 0 | |
| Glycopeptides | 10 (66.7%) | 13 (76.5%) | 0.699 |
| Antifolate | 1 (6.7%) | 3 (9.4%) | 0.603 |
| Nitroimidazoles | 0 | 1 (5.9%) | 1.000 |
| Polymyxin and Lipopeptides | 0 | 2 (6.3%) | 0.486 |

### Example 3. Patients Presenting with SE (of Known Cause or NORSE) Harbor Different Microbiomes than Patients with Chronic Epilepsy

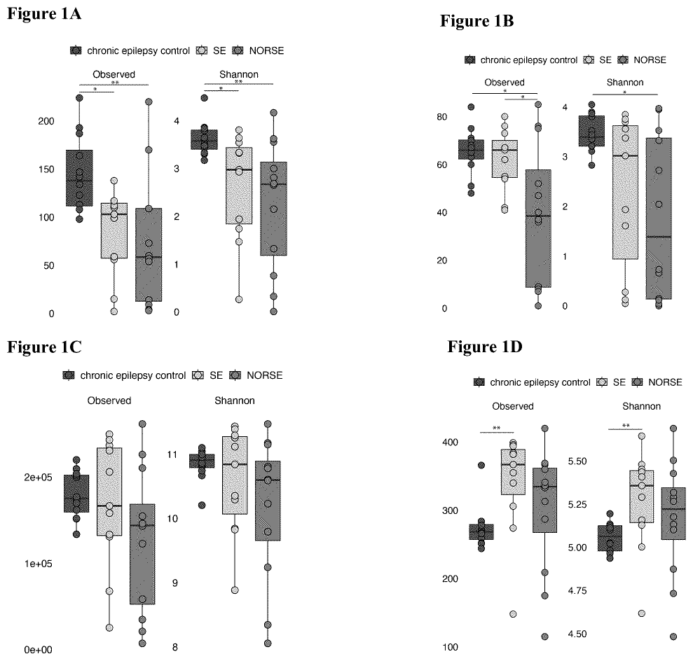

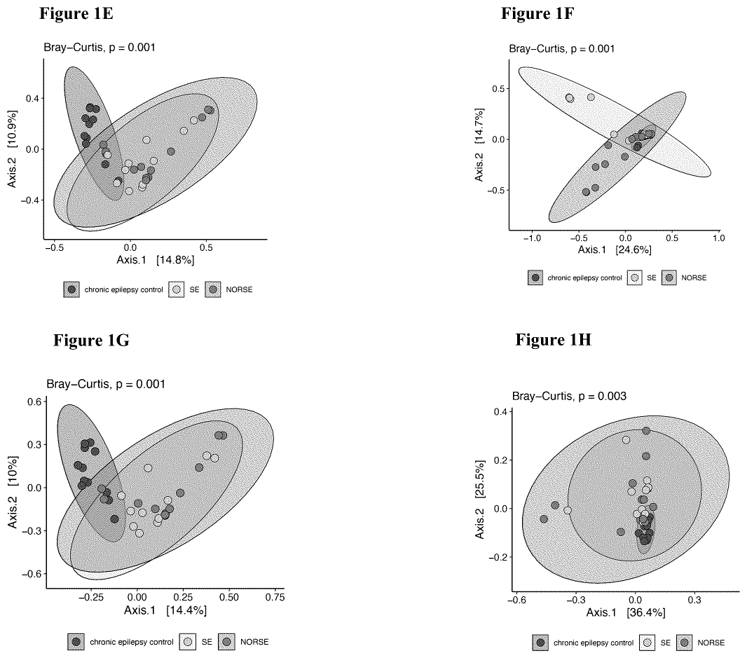

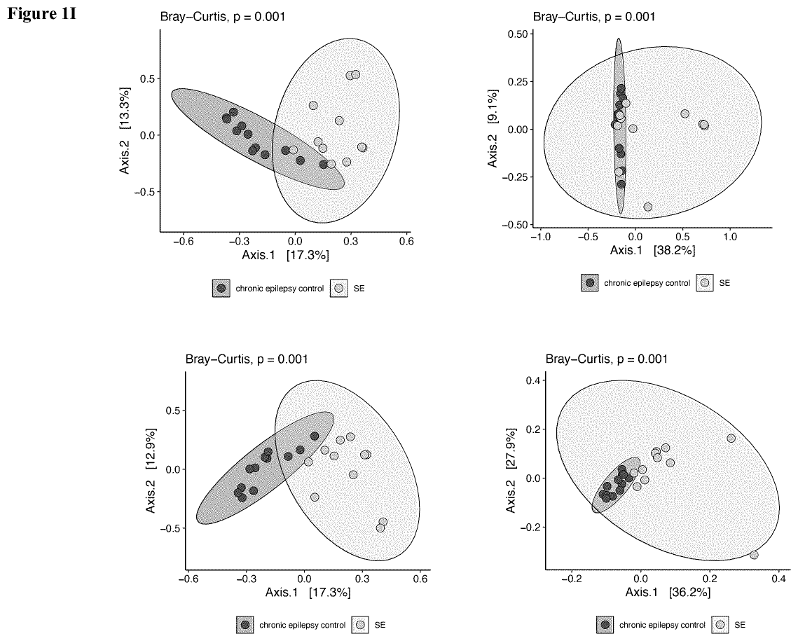

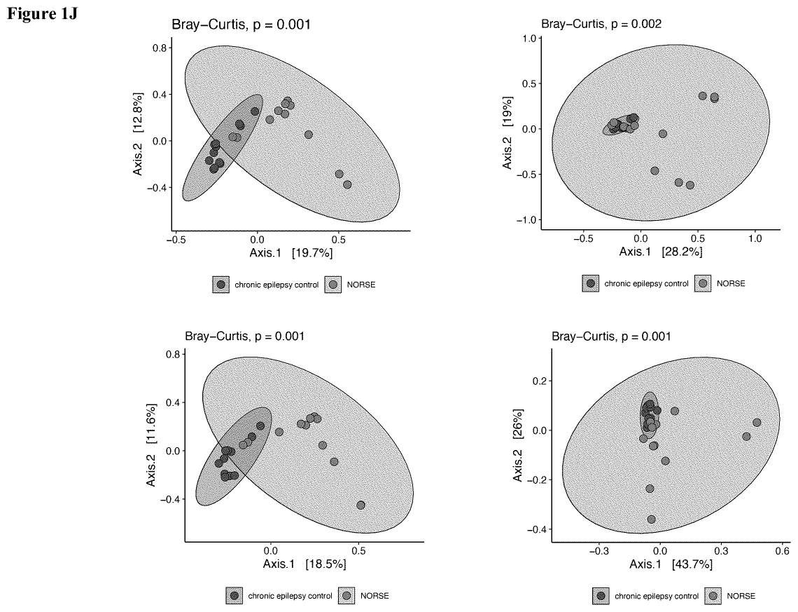

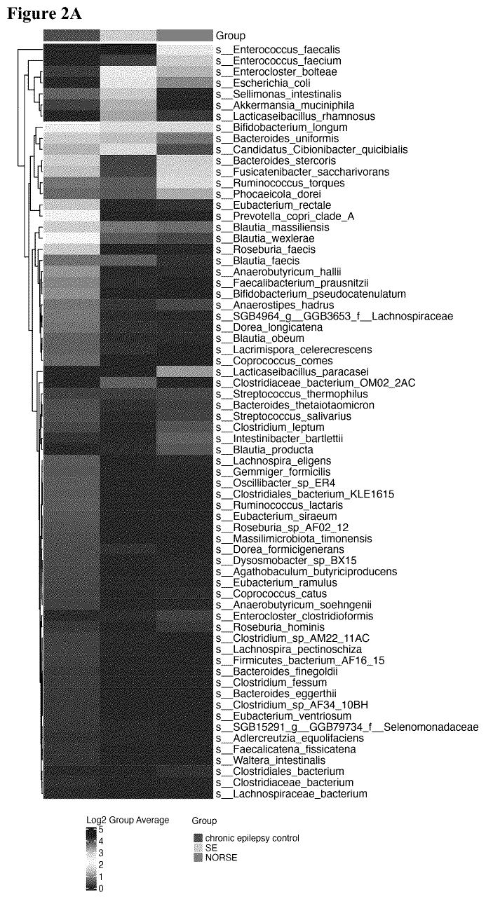

All SE patients (NORSE and SE of known cause) exhibited a significant difference in their fecal microbiome compared to otherwise healthy chronic epilepsy patients (FIG. 1A-1J). Alpha and beta diversity was evaluated for the taxonomic and functional composition of the given cohorts. Species-level taxonomic alpha diversity for prokaryotes and eukaryotes was significantly higher in the epilepsy control cohort than the NORSE cohort (FIGS. 1A and 1B), while only prokaryote alpha diversity was significantly higher than the SE cohort (FIG. 1A). Functional alpha diversity showed no significant differences at the gene family level (FIG. 1C), but at the pathway level the SE cohort had higher alpha diversity than the epilepsy control (FIG. 1D). No significant difference was found between the epilepsy control and NORSE cohorts despite notably higher average in alpha diversity measures for NORSE, likely due to the high degree of variance within the NORSE cohort (FIG. 1D). Microbiome beta diversity (Bray-Curtis, FIG. 1E-1H) significantly differed between cohorts, with pair-wise comparisons demonstrating NORSE and SE cohorts were significantly different than epilepsy controls across all levels (FIG. 1I-1J). In general, the variability within a cohort tended to be higher for SE and NORSE than the epilepsy control for both alpha and beta diversity.

Statistical analysis for the enrichment or depletion of specific microbiome features between the epilepsy control cohort and the SE or NORSE cohorts (T1) demonstrated species-level and functional-level differences (FIG. 2A-2C). The vast majority of taxonomic features that were found to be significantly differentially abundant were enriched in the chronic epilepsy control compared to the SE or NORSE cohorts (Prokaryotes 83% and 88%; eukaryotes 80% and 95%; respectively) (FIGS. 2 A and 2B), reflecting the lower alpha diversity in SE and NORSE. The Bacterial species Enterococcus faecalis and Escherichia coli were significantly enriched in both the SE and NORSE cohorts (FIG. 2A). Fusicatenibacter saccharivorans, Clostridium leptum, Lacticaseibacillus rhamnosus, Streptococcus anginosus, and Roseburia hominis were significantly enriched in the SE cohort (FIG. 2A). Enterocloster bolteae, and an unidentified Clostridiaceae species (bacterium_OM02_2AC) were enriched in the NORSE cohort (FIG. 2A). The eukaryotic order Saccharomycetales was enriched in both the SE and NORSE cohorts. The fungal species Nakaseomyces glabratus and Marasmius oreades were enriched in the NORSE cohort (FIG. 2B). No eukaryotic species were significantly enriched in the SE cohort, yet the genus Torulaspora was, compared to to the chronic epilepsy control.

### Example 4. SE and NORSE Patients Exhibit Limited Differences in Microbiome Diversity During Hospitalization

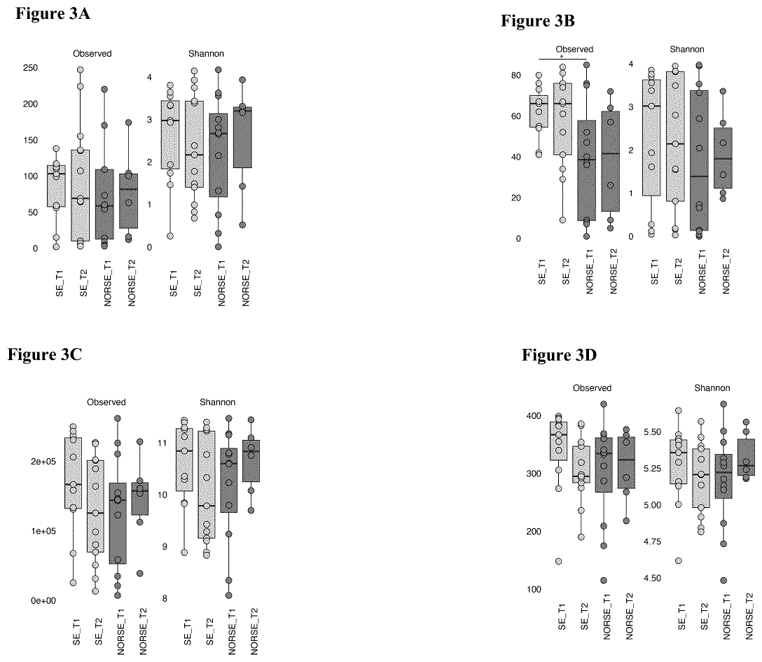

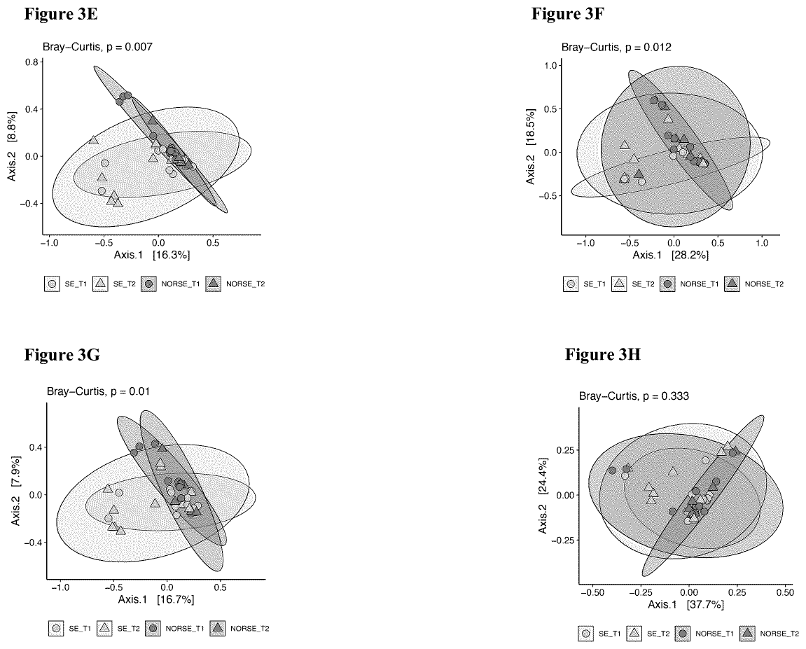

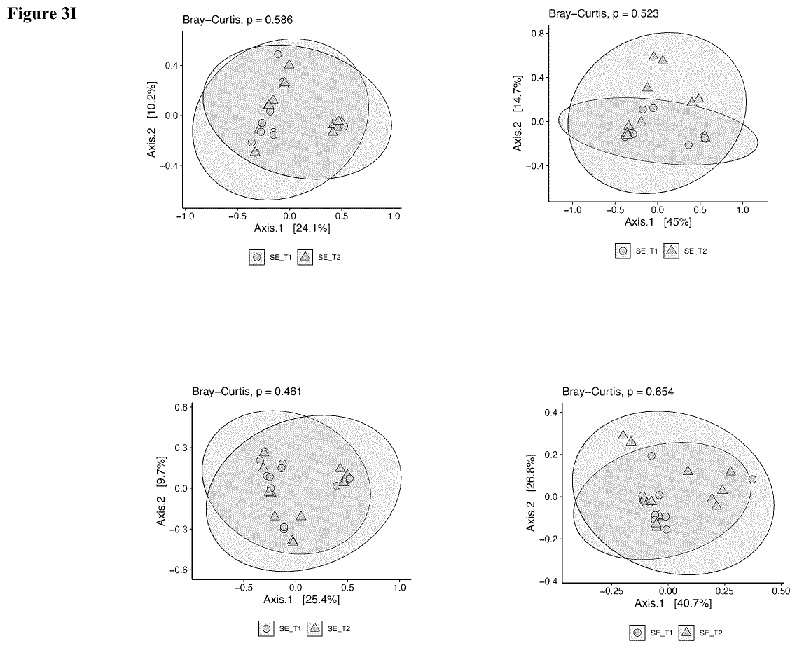

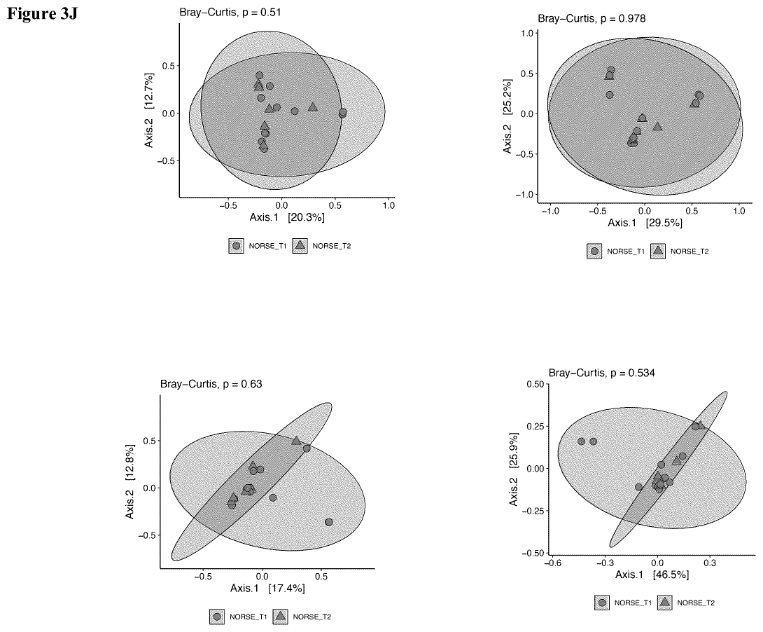

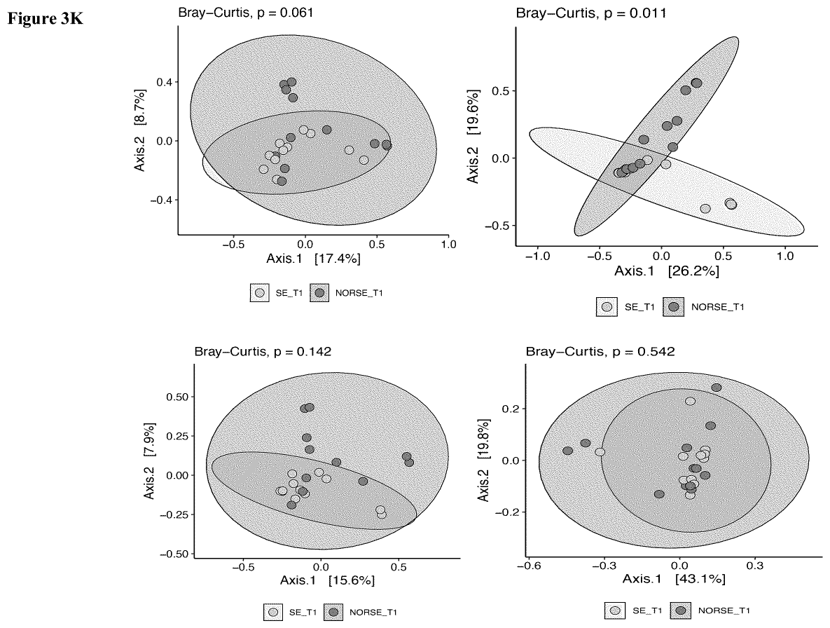

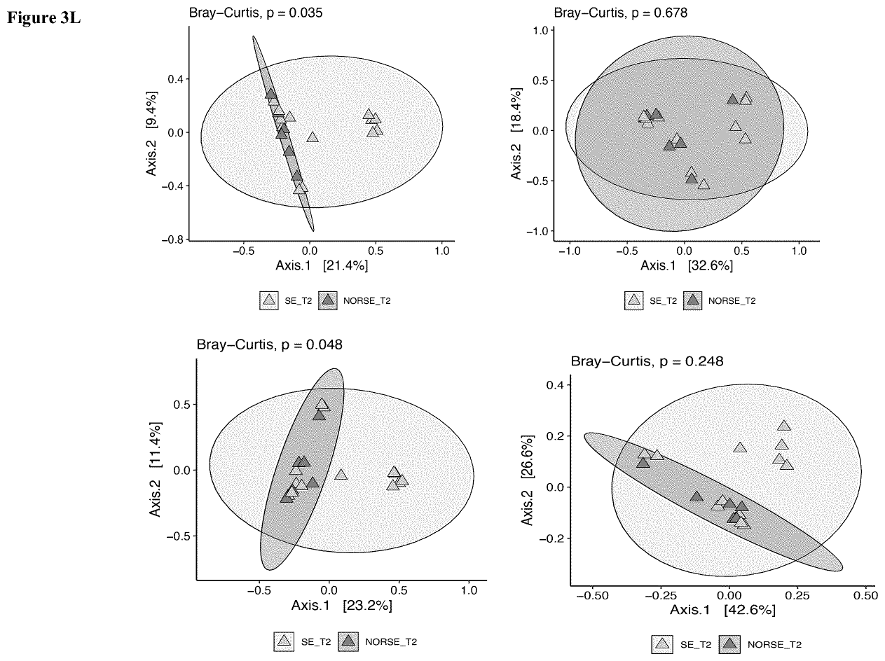

When evaluating diversity between and within the SE and NORSE cohorts, no significant differences in prokaryotic alpha diversity was observed (FIG. 3A), yet eukaryotic richness was significantly higher in SE during status (T1, FIG. 3B). Pathway level alpha diversity was not significantly different between or within the SE or NORSE cohorts (FIG. 3D). Prokaryotic community composition was significantly different 4 days or more after SE resolution between SE and NORSE patients (T2), but similar during or close to status (T1) (FIG. 3E). No significant differences in community composition were observed within each NORSE and SE cohort before or within 3 days (T1) and more than four days after (T2) SE resolution (FIG. 3I). The SE cohort harbored a significantly different eukaryotic microbial community than the NORSE cohort at T1 but became more similar after SE resolution (T2) (FIG. 3F). Within cohorts, the SE and NORSE patients did not significantly differ in eukaryotic microbial community structure based on time from status (FIG. 3J). A significant difference was observed in the functional microbiome composition at the gene family-level after status resolution between the SE and NORSE cohorts, but not within cohorts (FIGS. 3G and 3K). No significant differences in pathway-level functional microbiome composition were observed based on any comparisons for (FIGS. 3H and 3L), despite gene-family level differences found between cohorts at T2.

Statistical analysis for the enrichment or depletion of specific microbiome features between and within the SE or NORSE cohorts demonstrated limited species-level and functional-level differences (FIG. 4A-4C). Hierarchical clustering of the top 40 mean relative abundance prokaryotic species (FIG. 4A) showed a grouping of six taxa that were generally enriched in the NORSE cohort at both timepoints compared to the SE cohort, with Bacteroides ovatus and Sellimonas intestinalis being significantly enriched at T1 or T1 and T2 in the NORSE cohort, respectively. Akkermansia muciniphila was significantly enriched at T1 in NORSE, Staphylococcus aureus was significantly enriched at T2 compared to SE. The SE cohort had several species that were significantly different between T1 and T2 (i.e., Enterocloster bolteae, Clostridium butyricum, and Ruminococcus gnavus). Hierarchical clustering of the top 40 mean relative abundance eukaryotic species (FIG. 4B) showed differences between time groups within control cohort. The majority of significant differences found were from enriched species in the SE cohort compared to the NORSE cohort. Kluyvermoyces lactis, Talaromyces rugulosus, Naumovozyme dairenensis, and Talaromyces marneffei were enriched at T1, while Pichia kudriavzevii and Tetrapisispora phaffii were enriched at T2 in the SE cohort compared to the NORSE. Tetrapisispora blattae, Saccharomycodes ludwigii, and Nakaseomyces glabratus were enriched at T1 and T2 in the SE cohort compared to the NORSE. Malassezia restricta was enriched at T1 in SE compared to T2 in SE. Hierarchical clustering of the significantly different relative abundance pathways (FIG. 4C) showed difference between groups.

### Example 5. Microbiota Correlate with Plasma Cytokines Differently in the NORSE and SE Cohorts

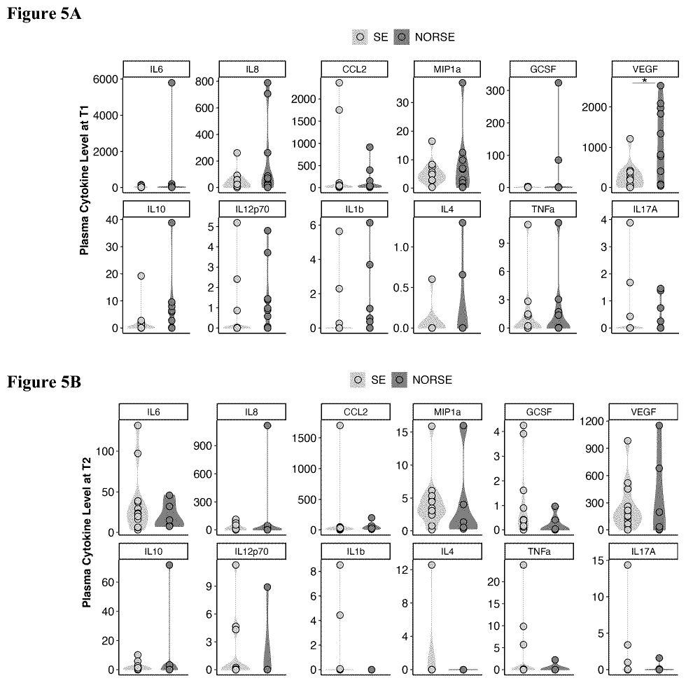

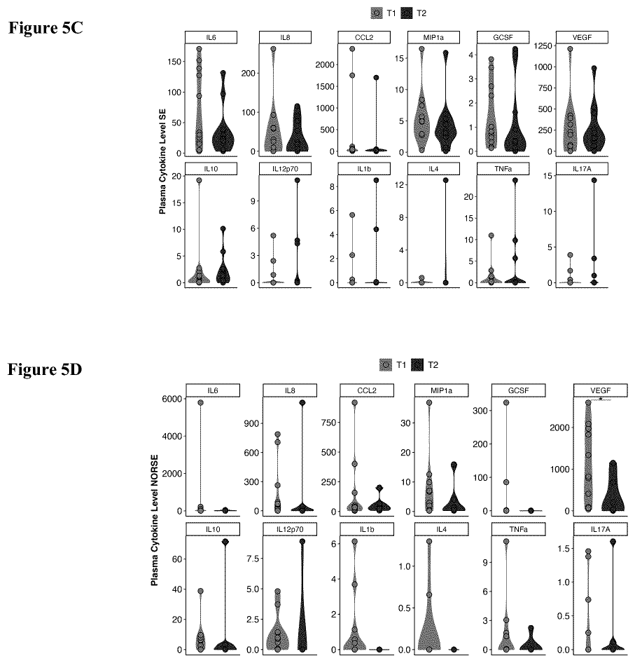

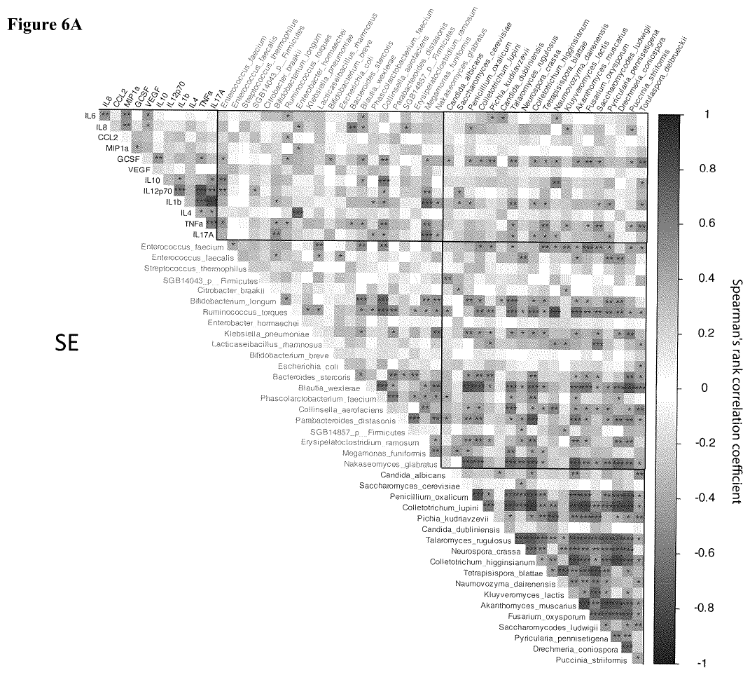

Subjects with NORSE had elevated levels of VEGF during or within 3 days of status epilepticus resolution (FIG. 5A), otherwise cytokine levels were no different between NORSE and status epilepticus control subjects (FIG. 5A-5B), and did not change over time, after status epilepticus resolution (FIG. 5C-5D).

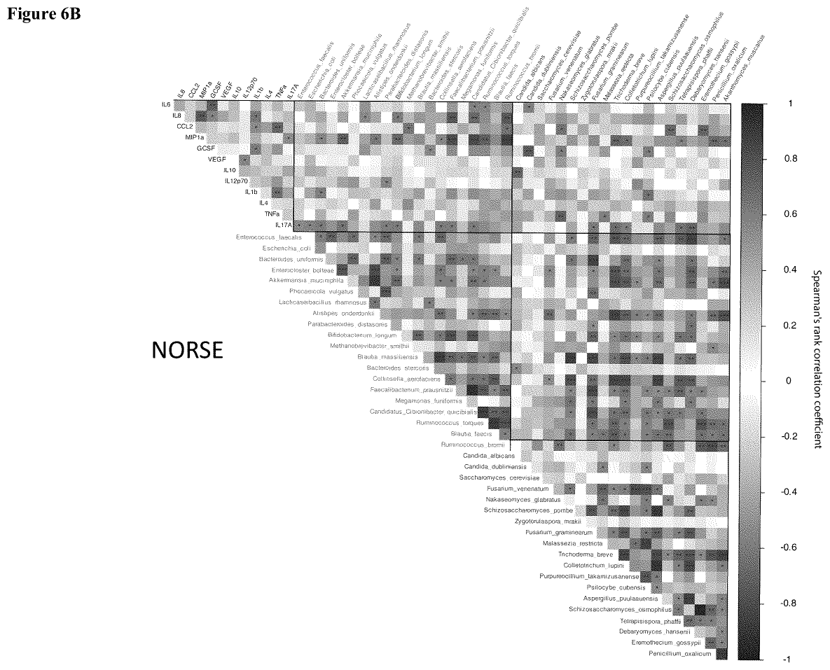

Spearman's rank correlation coefficients were calculated to assess the association between the relative abundance of the top 20 bacterial and eukaryotic species and plasma inflammatory cytokine levels (FIG. 6). The SE cohort (FIG. 6A) had more significantly positive correlations between cytokines than the NORSE cohort (FIG. 6B), with TNFa, IL12p70, and IL-17a correlating with each other and MIP1a, GCSF, IL1b, and IL4. Additionally, GCSF positively correlated with IL10 and IL8. In contrast, the NORSE cohort (FIG. 6B) only had two cytokines with more than 3 significant correlations to other cytokines (i.e., GCSF and IL12p70). The SE cohort also generally had more positive correlations between the topmost relatively abundant species and cytokines levels, especially within the eukaryotic grouping. In SE, GCSF, IL10, IL12p70, IL1b, IL4, TNFa, and IL17A had positive correlations with the majority of eukaryotic species, and to a lesser extent with the prokaryotic species. In SE, Enterobacter hormaechei positively correlated with all cytokines, many significantly so. In both SE and NORSE, C. dubliniensis had numerous significantly positive correlations with cytokines. SE had fewer positive correlations patterns between prokaryotic species, and between prokaryotic species and eukaryotic species than NORSE. Bifidobacterium longum, Collinsella aerofaciens, and Ruminococcus torques positively correlated with the majority of eukaryotic species, many significantly so, in both SE and NORSE. In SE, C. dubliniensis had positive or near zero correlations with all other eukaryotic taxa, yet in NORSE had negative or near zero correlations with all other eukaryotic taxa. In SE, N. glabratus negatively correlated with most other eukaryotic species, many significantly so, yet in NORSE N. glabratus positively correlated with most eukaryotic species.

In NORSE, E. bolteae and A. muciniphila both had significant correlations with IL6, CCL2, GCSF, and IL1b, with each other. In NORSE, IL10 significantly correlated with E. coli, N. glabratus, Fusarium venenatum, M. restricta, Purpureocillium takamizusanense, and Psilocybe cubensis. While limited in significance, IL4 had positive correlations with the majority of prokaryotic and eukaryotic species in NORSE. In NORSE, generally, more positive correlations were found between prokaryotic taxa than in the SE. In NORSE, Enterococcus faecalis notably had no correlation or was negatively correlated with almost all other species, yet in SE significantly positively correlated with the bacterium Lacticaseibacillus rhamnosus and the fungi Nakaseomyces glabratus.

In SE, GCSF, IL10, IL12p70, IL1b, IL4, TNFa, and IL17A had positive correlations with fungi species and can be used for targeted therapy in SE. Whereas in NORSE, E. bolteae and A. muciniphila significantly correlated with each other, and both E. bolteae and A. muciniphila had significant correlations with IL6, CCL2, GCSF, and IL1b. IL10 in NORSE, significantly correlated with E. coli, N. glabratus, Fusarium venenatum, M. restricta, Purpureocillium takamizusanense, and Psilocybe cubensis. Bacteria and fungi are both able to modulate cytokines and together, correlate and modulate cytokines and host response. Thus, these consortium of bacteria and fungi have great influence on cytokine production and host response. These correlations can be exploited for the development of new therapeutics for NORSE and SE. Microbiota can be modulated with pre or probiotics and antimicrobial/antifungal treatment. Elevated cytokines can be treated with immune system modulators, corticosteroids (e.g. dexamethasone), and/or biologics (e.g. Tumor necrosis factor (TNF) inhibitors, interleukin-1 (IL-1) inhibitors, and interleukin-6 (IL-6) inhibitors).

### Methods

Cytokine levels were obtained from serum, collected in plasma tube, in the NORSE subjects, and from plasma, collected in a 2 mL EDTA tube, in SE subjects. Samples were obtained shortly after seizure onset and longitudinally where possible, allowing for the observation of changes in patient microbiomes immediately after a seizure event and during prolonged hospitalization. Plasma samples were processed within 1 hour of collection by centrifuging at 2,000 rotations per minute for 20 minutes at 4° C. After separating plasma from red blood cells, plasma was stored at −80° C. until further processing.

Spearman's rank correlation coefficients were calculated to assess the association between the relative abundance of the top 20 bacterial, fungal species and plasma inflammatory cytokine levels in the samples using the Hmisc r package (v5.1-1). The correlation heatmap was generated using corrplot r package (v0.92). The differences in cytokine levels between the comparative groups were accessed by the Wilcoxon rank sum test. A mosaic plot was generated based on the results of Fisher's exact test to determine if there are any significant associations between the clinical outcomes and different SE presentations. Differences were considered as statistically significant at p value less than 0.05.

### Example 6. Microbiome Diversity and Tube Feeding Predict Mortality

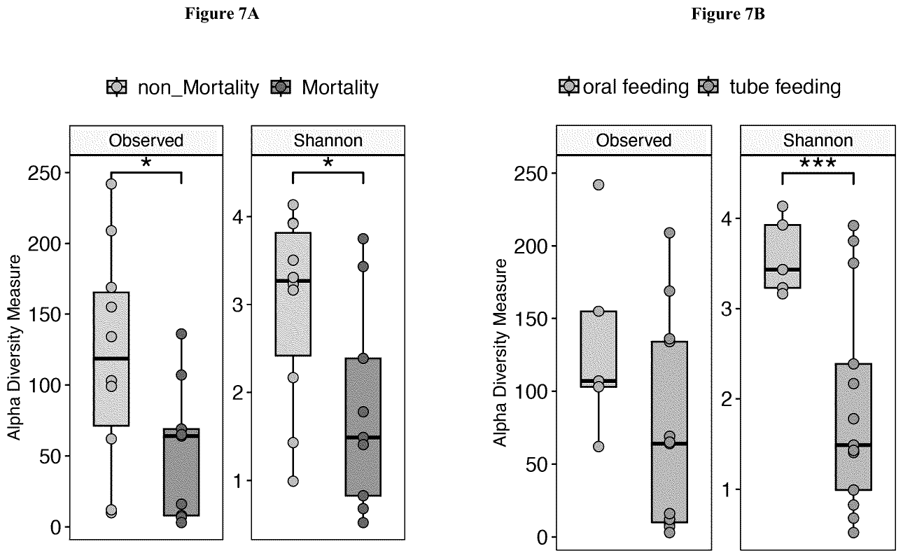

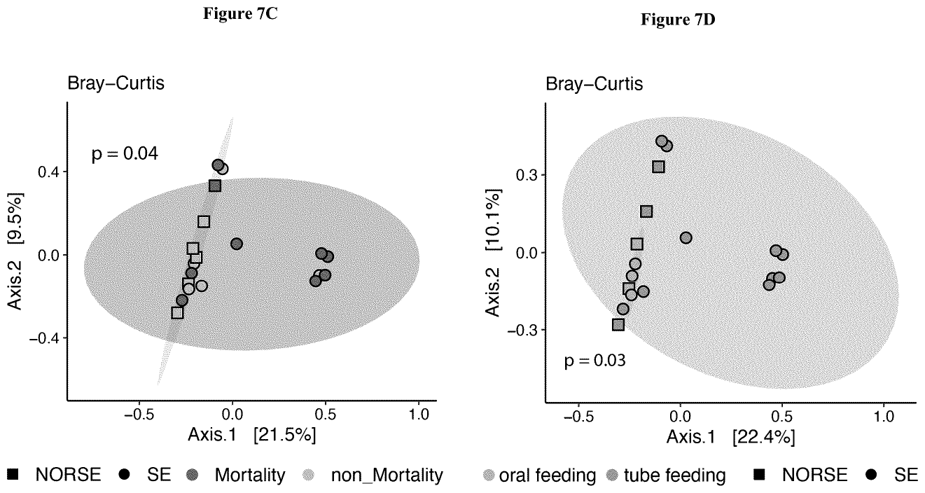

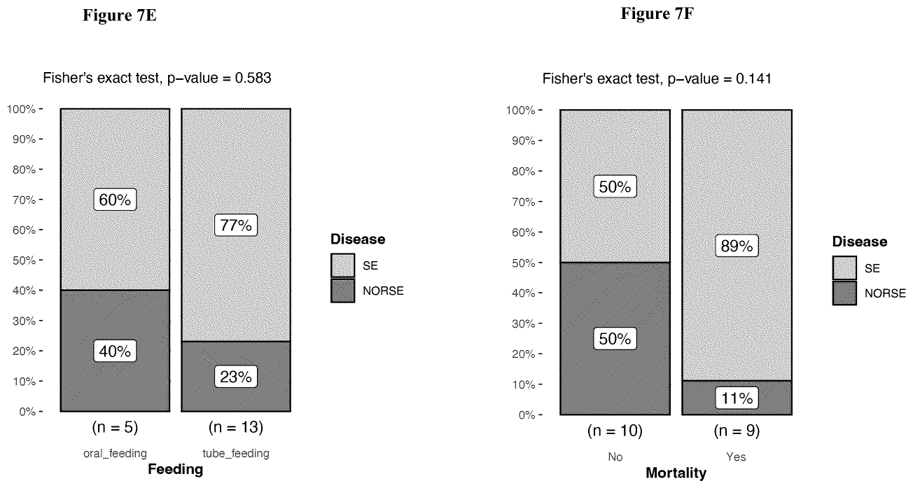

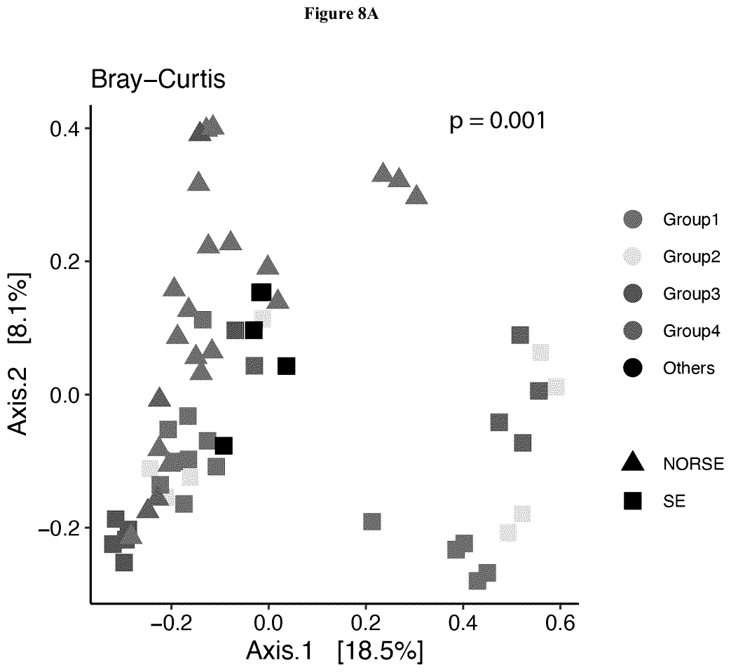

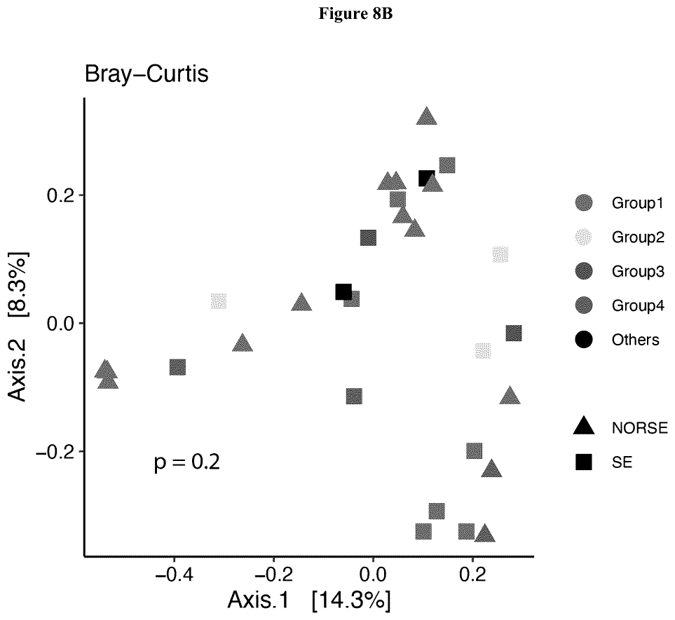

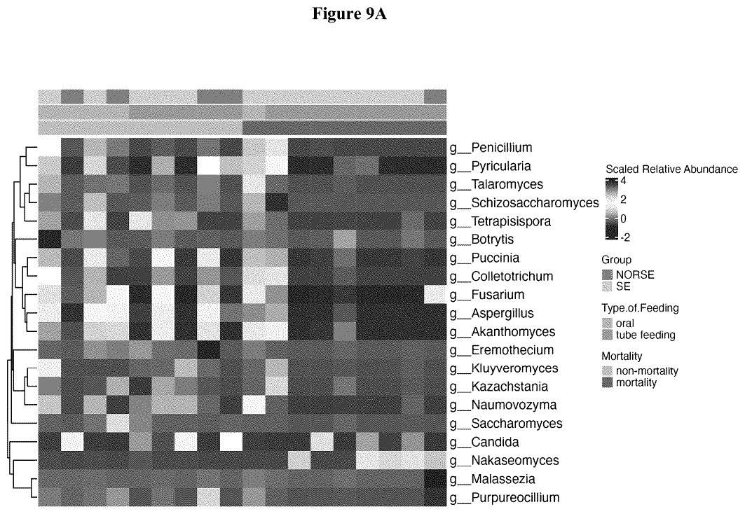

Alpha diversity and beta diversity differed between those with NORSE and SE of known cause who survived their illness at 3 months and those who died (FIG. 7A-7C). Similarly, those who were tube fed vs orally fed at the time of microbiome collection differed in both alpha and beta diversity. Beta diversity plots demonstrated similar clusters of similarity between those who survived and were orally fed. Prokaryotic alpha and beta diversity for mortality (FIG. 7A, 7C) and tube feeding (FIG. 7B, 7D) (latest T2 sample used). Between mortality outcomes, surviving patients harbored significantly higher prokaryotic species alpha diversity than patients who passed away (FIG. 7A). Between feeding outcomes, orally fed patients harbored significantly higher prokaryotic Shannon diversity than patients who passed away (FIG. 7B). Prokaryotic beta diversity analysis (species-level) indicates different microbial communities were associated with patients who passed away during the observation period and those who survived, and a greater degree of variance in community composition was noted for patients who passed away during the observation period (FIG. 7C). Prokaryotic beta diversity analysis (species-level) indicates different microbial communities were associated with patients who were tube fed than orally fed, and a greater degree of variance in community composition was noted for patients who were tube fed (FIG. 7D). Fisher's exact test shows the outcomes of feeding or mortality is not significantly associated with presenting with NORSE or SE (FIG. 7E-7F).

### Example 7

Microbiome features that are significantly enriched or depleted across or within SE and NORSE, oral and tube feeding, and mortality and non-mortality cohorts for Eukaryotes (FIG. 9A) and Prokaryotes (FIG. 10A), are noted. Top 40 species mean relative abundance heat maps (row z-score scaled) with hierarchical clustering based on species abundance patterns across cohorts and time points. Prokaryotic species significantly differentially abundant were observed Table 7. Significantly different species were observed in each cohort as observed by LEfSe pairwise comparisons. Fungal species are significantly differentially abundant. The data suggested that using microbiome data it is possible to determine the clinical parameters on oral or tube feed. The data further suggest that using fungi and bacterial data it is possible to identify patients who are at risk of high mortality.

TABLE 6
Differential abundance of Eukaryotes
(Fungi) in oral and tube feeding cohort

| | | enrich_ | | | |
|---|---|---|---|---|---|
| | feature | group | ef_lda | pvalue | padj |
| marker1 | p_Ascomycota\|c_ | oral | 4.901324 | 0.026251 | 0.026251 |
| | Eurotiomycetes | | | | |
| marker2 | p_Ascomycota\|c_ | oral | 4.901324 | 0.026251 | 0.026251 |
| | Eurotiomycetes\|o_ | | | | |
| | Eurotiales | | | | |
| marker3 | p_Ascomycota\|c_ | oral | 4.76282 | 0.026251 | 0.026251 |
| | Eurotiomycetes\|o_ | | | | |
| | Eurotiales\|f_ | | | | |
| | Aspergillaceae | | | | |
| marker4 | p_Ascomycota\|c_ | oral | 4.749194 | 0.034064 | 0.034064 |
| | Sordariomycetes\|o_ | | | | |
| | Hypocreales\|f_ | | | | |
| | Nectriaceae | | | | |
| marker5 | p_Ascomycota\|c_ | oral | 4.749194 | 0.034064 | 0.034064 |
| | Sordariomycetes\|o_ | | | | |
| | Hypocreales\|f_ | | | | |
| | Nectriaceae\|g_ | | | | |
| | Fusarium | | | | |
| marker6 | p_Ascomycota\|c_ | oral | 4.420285 | 0.025017 | 0.025017 |
| | Eurotiomycetes\|o_ | | | | |
| | Eurotiales\|f_ | | | | |
| | Trichocomaceae | | | | |
| marker7 | p_Ascomycota\|c_ | oral | 4.420285 | 0.025017 | 0.025017 |
| | Eurotiomycetes\|o_ | | | | |
| | Eurotiales\|f_ | | | | |
| | Trichocomaceae\|g_ | | | | |
| | Talaromyces | | | | |
| marker8 | p_Ascomycota\|c_ | oral | 4.398423 | 0.037361 | 0.037361 |
| | Eurotiomycetes\|o_ | | | | |
| | Eurotiales\|f_ | | | | |
| | Aspergillaceae\|g_ | | | | |
| | Aspergillus\|s_ | | | | |
| | Aspergillus_ | | | | |
| | chevalieri | | | | |
| marker9 | p_Ascomycota\|c_ | oral | 4.319074 | 0.039122 | 0.039122 |
| | Eurotiomycetes\|o_ | | | | |
| | Eurotiales\|f_ | | | | |
| | Trichocomaceae\|g_ | | | | |
| | Talaromyces\|s_ | | | | |
| | Talaromyces_ | | | | |
| | marneffei | | | | |
| marker10 | p_Ascomycota\|c_ | oral | 4.254971 | 0.025017 | 0.025017 |
| | Sordariomycetes\|o_ | | | | |
| | Hypocreales\|f_ | | | | |
| | Clavicipitaceae | | | | |
| marker11 | p_Ascomycota\|c_ | oral | 4.242585 | 0.008301 | 0.008301 |
| | Eurotiomycetes\|o_ | | | | |
| | Eurotiales\|f_ | | | | |
| | Aspergillaceae\| | | | | |
| | g_Penicillium | | | | |
| marker12 | p_Ascomycota\|c_ | oral | 4.242585 | 0.008301 | 0.008301 |
| | Eurotiomycetes\|o_ | | | | |
| | Eurotiales\|f_ | | | | |
| | Aspergillaceae\|g_ | | | | |
| | Penicillium\|s_ | | | | |
| | Penicillium_ | | | | |
| | oxalicum | | | | |
| marker13 | p_Ascomycota\|c_ | oral | 4.208264 | 0.020273 | 0.020273 |
| | Saccharomycetes\|o_ | | | | |
| | Saccharo- | | | | |
| | mycetales\|f_ | | | | |
| | Pichiaceae | | | | |
| marker14 | p_Basidiomycota\|c_ | oral | 4.180085 | 0.043198 | 0.043198 |
| | Pucciniomycetes | | | | |
| marker15 | p_Basidiomycota\|c_ | oral | 4.180085 | 0.043198 | 0.043198 |
| | Pucciniomycetes\|o_ | | | | |
| | Pucciniales | | | | |
| marker16 | p_Basidiomycota\|c_ | oral | 4.180085 | 0.043198 | 0.043198 |
| | Pucciniomycetes\|o_ | | | | |
| | Pucciniales\|f_ | | | | |
| | Pucciniaceae | | | | |
| marker17 | p_Basidiomycota\|c_ | oral | 4.180085 | 0.043198 | 0.043198 |
| | Pucciniomycetes\|o_ | | | | |
| | Pucciniales\|f_ | | | | |
| | Pucciniaceae\|g_ | | | | |
| | Puccinia | | | | |
| marker18 | p_Ascomycota\|c_ | oral | 4.130817 | 0.030308 | 0.030308 |
| | Sordariomycetes\|o_ | | | | |
| | Hypocreales\|f_ | | | | |
| | Clavicipitaceae\|g_ | | | | |
| | Ustilaginoidea | | | | |
| marker19 | p_Ascomycota\|c_ | oral | 4.130817 | 0.030308 | 0.030308 |
| | Sordariomycetes\|o_ | | | | |
| | Hypocreales\|f_ | | | | |
| | Clavicipitaceae\|g_ | | | | |
| | Ustilaginoidea\|s_ | | | | |
| | Ustilaginoidea_ | | | | |
| | virens | | | | |
| marker20 | p_Ascomycota\|c_ | oral | 4.120609 | 0.041159 | 0.041159 |
| | Dothideomycetes | | | | |
| marker21 | p_Ascomycota\|c_ | oral | 4.120609 | 0.041159 | 0.041159 |
| | Dothideomycetes\|o_ | | | | |
| | Mycosphaerellales | | | | |
| marker22 | p_Ascomycota\|c_ | oral | 4.120609 | 0.041159 | 0.041159 |
| | Dothideomycetes\|o_ | | | | |
| | Myco- | | | | |
| | sphaerellales\|f_ | | | | |
| | Mycosphaerellaceae | | | | |
| marker23 | p_Ascomycota\|c_ | oral | 4.101627 | 0.002382 | 0.002382 |
| | Sordariomycetes\|o_ | | | | |
| | Hypocreales\|f_ | | | | |
| | Hypocreaceae | | | | |
| marker24 | p_Ascomycota\|c_ | oral | 4.101627 | 0.002382 | 0.002382 |
| | Sordariomycetes\|o_ | | | | |
| | Hypocreales\|f_ | | | | |
| | Hypocreaceae\|g_ | | | | |
| | Trichoderma | | | | |
| marker25 | p_Ascomycota\|c_ | oral | 4.101627 | 0.002382 | 0.002382 |
| | Sordariomycetes\|o_ | | | | |
| | Hypocreales\|f_ | | | | |
| | Hypocreaceae\|g_ | | | | |
| | Trichoderma\|s_ | | | | |
| | Trichoderma_breve | | | | |
| marker26 | p_Ascomycota\|c_ | oral | 4.092585 | 0.019245 | 0.019245 |
| | Sordariomycetes\|o_ | | | | |
| | Hypocreales\|f_ | | | | |
| | Nectriaceae\|g_ | | | | |
| | Fusarium\|s_ | | | | |
| | Fusarium_ | | | | |
| | oxysporum | | | | |
| marker27 | p_Basidiomycota\|c_ | oral | 4.083963 | 0.019885 | 0.019885 |
| | Agaricomycetes | | | | |
| marker28 | p_Basidiomycota\|c_ | oral | 4.073114 | 0.011906 | 0.011906 |
| | Pucciniomycetes\|o_ | | | | |
| | Pucciniales\|f_ | | | | |
| | Pucciniaceae\|g_ | | | | |
| | puccinia\|s_ | | | | |
| | Puccinia_ | | | | |
| | striiformis | | | | |
| marker29 | p_Ascomycota\|c_ | oral | 3.989981 | 0.030863 | 0.030863 |
| | Saccharomycetes\|o_ | | | | |
| | Saccharo- | | | | |
| | mycetales\|f_ | | | | |
| | Saccharo- | | | | |
| | mycetaceae\|g_ | | | | |
| | Eremothecium\|s_ | | | | |
| | Eremothecium_ | | | | |
| | cymbalariae | | | | |
| marker30 | p_Basidiomycota\|c_ | oral | 3.983698 | 0.008301 | 0.008301 |
| | Agaricomycetes\|o_ | | | | |
| | Agaricales\|f_ | | | | |
| | Marasmiaceae | | | | |
| marker31 | p_Basidiomycota\|c_ | oral | 3.983698 | 0.008301 | 0.008301 |
| | Agaricomycetes\|o_ | | | | |
| | Agaricales\|f_ | | | | |
| | Marasmiaceae\|g_ | | | | |
| | Marasmius | | | | |
| marker32 | p_Basidiomycota\|c_ | oral | 3.983698 | 0.008301 | 0.008301 |
| | Agaricomycetes\|o_ | | | | |
| | Agaricales\|f_ | | | | |
| | Marasmiaceae\|g_ | | | | |
| | Marasmius\|s_ | | | | |
| | Marasmius_oreades | | | | |
| marker33 | p_Ascomycota\|c_ | oral | 3.92126 | 0.013899 | 0.013899 |
| | Sordariomycetes\|o_ | | | | |
| | Hypocreales\|f_ | | | | |
| | Nectriaceae\|g_ | | | | |
| | Fusarium\|s_ | | | | |
| | Fusarium_fujikuroi | | | | |
| marker34 | p_Basidiomycota\|c_ | oral | 3.90854 | 0.025786 | 0.025786 |
| | Agaricomycetes\|o_ | | | | |
| | Agaricales | | | | |
| marker35 | p_Ascomycota\|c_ | oral | 3.900035 | 0.042875 | 0.042875 |
| | Sordariomycetes\|o_ | | | | |
| | Hypocreales\|f_ | | | | |
| | Ophio- | | | | |
| | cordycipitaceae\|g_ | | | | |
| | Purpureocillium | | | | |
| marker36 | p_Ascomycota\|c_ | oral | 3.900035 | 0.042875 | 0.042875 |
| | Sordariomycetes\|o_ | | | | |
| | Hypocreales\|f_ | | | | |
| | Ophio- | | | | |
| | cordycipitaceae\|g_ | | | | |
| | Purpureocillium\|s_ | | | | |
| | Purpureocillium_ | | | | |
| | takamizusanense | | | | |
| marker37 | p_Basidiomycota\|c_ | oral | 3.893689 | 0.039566 | 0.039566 |
| | Ustilaginomycetes | | | | |
| marker38 | p_Basidiomycota\|c_ | oral | 3.893689 | 0.039566 | 0.039566 |
| | Ustilagino- | | | | |
| | mycetes\|o_ | | | | |
| | Ustilaginales | | | | |
| marker39 | p_Basidiomycota\|c_ | oral | 3.893689 | 0.039566 | 0.039566 |
| | Ustilagino- | | | | |
| | mycetes\|o_ | | | | |
| | Ustilaginales\|f_ | | | | |
| | Ustilaginaceae | | | | |
| marker40 | p_Ascomycota\|c_ | oral | 3.748613 | 0.030308 | 0.030308 |
| | Sordariomycetes\|o_ | | | | |
| | Hypocreales\|f_ | | | | |
| | Nectriaceae\|g_ | | | | |
| | Fusarium\|s_ | | | | |
| | Fusarium_poae | | | | |
| marker41 | p_Ascomycota\|c_ | oral | 3.739688 | 0.023054 | 0.023054 |
| | Sordariomycetes\|o_ | | | | |
| | Hypocreales\|f_ | | | | |
| | Nectriaceae\|g_ | | | | |
| | Fusarium\|s_ | | | | |
| | Fusarium_ | | | | |
| | graminearum | | | | |
| marker42 | p_Ascomycota\|c_ | oral | 3.667245 | 0.030877 | 0.030877 |
| | Dothideomycetes\|o_ | | | | |
| | Mycosphaerellales\|f_ | | | | |
| | Myco- | | | | |
| | sphaerellaceae\|g_ | | | | |
| | Zymoseptoria | | | | |
| marker43 | p_Ascomycota\|c_ | oral | 3.667245 | 0.030877 | 0.030877 |
| | Dothideomycetes\|o_ | | | | |
| | Myco- | | | | |
| | sphaerellales\|f_ | | | | |
| | Myco- | | | | |
| | sphaerellaceae\|g_ | | | | |
| | Zymoseptoria\|s_ | | | | |
| | Zymoseptoria_ | | | | |
| | tritici | | | | |
| marker44 | p_Basidiomycota\|c_ | tube | 4.274731 | 0.049753 | 0.049753 |
| | Malassezio- | feeding | | | |
| | mycetes\|o_ | | | | |
| | Malasseziales\|f_ | | | | |
| | Malasseziaceae\|g_ | | | | |
| | Malassezia\|s_ | | | | |
| | Malassezia_ | | | | |
| | restricta | | | | |
| marker45 | p_Ascomycota\|c_ | tube | 4.095371 | 0.032231 | 0.032231 |
| | Eurotiomycetes\|o_ | feeding | | | |
| | Eurotiales\|f_ | | | | |
| | Trichocomaceae\|g_ | | | | |
| | Talaromyces\|s_ | | | | |
| | Talaromyces_ | | | | |
| | rugulosus | | | | |
| marker46 | p_Ascomycota\|c_ | tube | 3.569065 | 0.03044 | 0.03044 |
| | Saccharomycetes\|o_ | feeding | | | |
| | Saccharo- | | | | |
| | mycetales\|f_ | | | | |
| | Debaryo- | | | | |
| | mycetaceae\|g_ | | | | |
| | Scheffersomyces\|s_ | | | | |
| | Scheffersomyces_ | | | | |
| | stipitis | | | | |
| marker47 | p_Ascomycota\|c_ | tube | 3.569065 | 0.03044 | 0.03044 |
| | Saccharomycetes\|o_ | feeding | | | |
| | Saccharo- | | | | |
| | mycetales\|f_ | | | | |
| | Debaryomycetaceae\| | | | | |
| | g_Scheffersomyces | | | | |
| marker48 | p_Ascomycota\|c_ | tube | 3.167093 | 0.03044 | 0.03044 |
| | Saccharomycetes\|o_ | feeding | | | |
| | Saccharo- | | | | |
| | mycetales\|f_ | | | | |
| | Saccharo- | | | | |
| | mycetaceae\|g_ | | | | |
| | Lachancea | | | | |
| marker49 | p_Ascomycota\|c_ | tube | 3.167093 | 0.03044 | 0.03044 |
| | Saccharomycetes\|o_ | feeding | | | |
| | Saccharo- | | | | |
| | mycetales\|f_ | | | | |
| | Saccharo- | | | | |
| | mycetaceae\|g_ | | | | |
| | Lachancea\|s_ | | | | |
| | Lachancea_ | | | | |
| | thermotolerans | | | | |

TABLE 7
Differential abundance of Prokaryotes
(bacteria) in oral and tube feeding cohort

| | | enrich_ | | | |
|---|---|---|---|---|---|
| | feature | group | ef_lda | pvalue | padj |
| marker1 | p_Firmicutes\|c_ | oral | 5.485262 | 0.011515 | 0.011515 |
| | Clostridia | | | | |
| marker2 | p_Firmicutes\|c_ | oral | 5.461207 | 0.015199 | 0.015199 |
| | Clostridia\|o_ | | | | |
| | Eubacteriales | | | | |
| marker3 | p_Bacteroidetes\|c_ | oral | 5.134939 | 0.033695 | 0.033695 |
| | Bacteroidia | | | | |
| marker4 | p_Bacteroidetes\|c_ | oral | 5.134939 | 0.033695 | 0.033695 |
| | Bacteroidia\|o_ | | | | |
| | Bacteroidales | | | | |
| marker5 | p_Bacteroidetes | oral | 5.13324 | 0.033695 | 0.033695 |
| marker6 | p_Bacteroidetes\|c_ | oral | 5.044232 | 0.015519 | 0.015519 |
| | Bacteroidia\|o_ | | | | |
| | Bacteroidales\|f_ | | | | |
| | Bacteroidaceae | | | | |
| marker7 | p_Bacteroidetes\|c_ | oral | 4.962069 | 0.042875 | 0.042875 |
| | Bacteroidia\|o_ | | | | |
| | Bacteroidales\|f_ | | | | |
| | Bacteroidaceae\|g_ | | | | |
| | Bacteroides | | | | |
| marker8 | p_Firmicutes\|c_ | oral | 4.956086 | 0.013899 | 0.013899 |
| | Clostridia\|o_ | | | | |
| | Eubacteriales\|f_ | | | | |
| | Oscillospiraceae | | | | |
| marker9 | p_Firmicutes\|c_ | oral | 4.861954 | 0.040022 | 0.040022 |
| | Clostridia\|o_ | | | | |
| | Eubacteriales\|f_ | | | | |
| | Lachnospiraceae\|g_ | | | | |
| | Mediter- | | | | |
| | raneibacter | | | | |
| marker10 | p_Firmicutes\|c_ | oral | 4.76602 | 0.023565 | 0.023565 |
| | Clostridia\|o_ | | | | |
| | Eubacteriales\|f_ | | | | |
| | Oscillospiraceae\|g_ | | | | |
| | Faecalibacterium | | | | |
| marker11 | p_Firmicutes\|c_ | oral | 4.755085 | 0.023565 | 0.023565 |
| | Clostridia\|o_ | | | | |
| | Eubacteriales\|f_ | | | | |
| | Oscillospiraceae\|g_ | | | | |
| | Faecalibacterium\| | | | | |
| | s_Faecali- | | | | |
| | bacterium_ | | | | |
| | prausnitzii | | | | |
| marker12 | p_Firmicutes\|c_ | oral | 4.455694 | 0.013899 | 0.013899 |
| | Erysipelotrichia\|o_ | | | | |
| | Erysipelo- | | | | |
| | trichales\|f_ | | | | |
| | Erysipelo- | | | | |
| | trichaceae | | | | |
| marker13 | p_Firmicutes\|c_ | oral | 4.450296 | 0.014673 | 0.014673 |
| | Clostridia\|o_ | | | | |
| | Eubacteriales\|f_ | | | | |
| | Clostridiaceae | | | | |
| marker14 | p_Firmicutes\|c_ | oral | 4.447378 | 0.013899 | 0.013899 |
| | Erysipelotrichia | | | | |
| marker15 | p_Firmicutes\|c_ | oral | 4.447378 | 0.013899 | 0.013899 |
| | Erysipelotrichia\|o_ | | | | |
| | Erysipelotrichales | | | | |
| marker16 | p_Actinobacteria\| | oral | 4.424775 | 0.044291 | 0.044291 |
| | c_Coriobacteriia\| | | | | |
| | o_Corio- | | | | |
| | bacteriales\|f_ | | | | |
| | Coriobacteriaceae | | | | |
| marker17 | p_Firmicutes\|c_ | oral | 4.313391 | 0.023054 | 0.023054 |
| | Clostridia\|o_ | | | | |
| | Clostridiales | | | | |
| marker18 | p_Firmicutes\|c_ | oral | 4.312399 | 0.012285 | 0.012285 |
| | Clostridia\|o_ | | | | |
| | Clostridiales\|f_ | | | | |
| | Ruminococcaceae | | | | |
| marker19 | p_Firmicutes\|c_ | oral | 4.310329 | 0.012285 | 0.012285 |
| | Clostridia\|o_ | | | | |
| | Clostridiales\|f_ | | | | |
| | Rumino- | | | | |
| | coccaceae\|g_ | | | | |
| | Candidatus_ | | | | |
| | Cibionibacter | | | | |
| marker20 | p_Firmicutes\|c_ | oral | 4.307553 | 0.012285 | 0.012285 |
| | Clostridia\|o_ | | | | |
| | Clostridiales\|f_ | | | | |
| | Rumino- | | | | |
| | coccaceae\|g_ | | | | |
| | Candidatus_ | | | | |
| | Cibionibacter\|s_ | | | | |
| | Candidatus_ | | | | |
| | Cibionibacter_ | | | | |
| | quicibialis | | | | |
| marker21 | p_Bacteroidetes\|c_ | oral | 4.220718 | 0.003372 | 0.003372 |
| | Bacteroidia\|o_ | | | | |
| | Bacteroidales\|f_ | | | | |
| | Bacteroidaceae\|g_ | | | | |
| | Bacteroides\|s_ | | | | |
| | Bacteroides_ | | | | |
| | xylanisolvens | | | | |
| marker22 | p_Firmicutes\|c_ | oral | 4.217208 | 0.037361 | 0.037361 |
| | Clostridia\|o_ | | | | |
| | Eubacteriales\|f_ | | | | |
| | Clostridiaceae\|g_ | | | | |
| | Clostridium | | | | |
| marker23 | p_Firmicutes\|c_ | oral | 4.207525 | 0.029026 | 0.029026 |
| | Clostridia\|o_ | | | | |
| | Eubacteriales\|f_ | | | | |
| | Lachnospiraceae\|g_ | | | | |
| | Roseburia | | | | |
| marker24 | p_Firmicutes\|c_ | oral | 4.206222 | 0.038061 | 0.038061 |
| | Negativicutes\|o_ | | | | |
| | Veillonellales\|f_ | | | | |
| | Veillonellaceae | | | | |
| marker25 | p_Firmicutes\|c_ | oral | 4.204478 | 0.038061 | 0.038061 |
| | Negativicutes\|o_ | | | | |
| | Veillonellales | | | | |
| marker26 | p_Firmicutes_c_ | oral | 4.204363 | 0.028793 | 0.028793 |
| | Erysipelotrichia\|o_ | | | | |
| | Erysipelo- | | | | |
| | trichales\|f_ | | | | |
| | Erysipelo- | | | | |
| | trichaceae\|g_ | | | | |
| | Erysipelato- | | | | |
| | clostridium\|s_ | | | | |
| | Erysipelato- | | | | |
| | clostridium_ | | | | |
| | ramosum | | | | |
| marker27 | p_Firmicutes\|c_ | oral | 4.203102 | 0.028793 | 0.028793 |
| | Erysipelotrichia\|o_ | | | | |
| | Erysipelo- | | | | |
| | trichales\|f_ | | | | |
| | Erysipelo- | | | | |
| | trichaceae\|g_ | | | | |
| | Erysipelato- | | | | |
| | clostridium | | | | |
| marker28 | p_Bacteroidetes\|c_ | oral | 4.196921 | 0.000965 | 0.000965 |
| | Bacteroidia\|o_ | | | | |
| | Bacteroidales\|f_ | | | | |
| | Odoribacteraceae\| | | | | |
| | g_Odoribacter | | | | |
| marker29 | p_Bacteroidetes\|c_ | oral | 4.19682 | 0.000965 | 0.000965 |
| | Bacteroidia\|o_ | | | | |
| | Bacteroidales\|f_ | | | | |
| | Odoribacteraceae\| | | | | |
| | g_Odoribacter\|s_ | | | | |
| | Odoribacter_ | | | | |
| | splanchnicus | | | | |
| marker30 | p_Bacteroidetes\|c_ | oral | 4.17949 | 0.013333 | 0.013333 |
| | Bacteroidia\|o_ | | | | |
| | Bacteroidales\|f_ | | | | |
| | Odoribacteraceae | | | | |
| marker31 | p_Bacteroidetes\| | oral | 4.151635 | 0.012285 | 0.012285 |
| | c_Bacteroidia\|o_ | | | | |
| | Bacteroidales\|f_ | | | | |
| | Bacteroidaceae\|g_ | | | | |
| | Bacteroides\|s_ | | | | |
| | Bacteroides_ | | | | |
| | stercoris | | | | |
| marker32 | p_Firmicutes\|c_ | oral | 4.14763 | 0.012285 | 0.012285 |
| | Clostridia\|o_ | | | | |
| | Eubacteriales\|f_ | | | | |
| | Lachnospiraceae\|g_ | | | | |
| | Roseburials_ | | | | |
| | Roseburia_ | | | | |
| | intestinalis | | | | |
| marker33 | p_Firmicutes\|c_ | oral | 4.110457 | 0.013333 | 0.013333 |
| | Clostridia\|o_ | | | | |
| | Eubacteriales\|f_ | | | | |
| | Clostridiaceae\|g_ | | | | |
| | Clostridium\|s_ | | | | |
| | Clostridium_ | | | | |
| | butyricum | | | | |
| marker34 | p_Bacteroidetes\|c_ | oral | 4.067076 | 0.039554 | 0.039554 |
| | Bacteroidia\|o_ | | | | |
| | Bacteroidales\|f_ | | | | |
| | Tannerellaceae\|g_ | | | | |
| | Parabacteroides\|s_ | | | | |
| | Parabacteroides_ | | | | |
| | merdae | | | | |
| marker35 | p_Firmicutes\|c_ | oral | 4.048935 | 0.01786 | 0.01786 |
| | Negativicutes\|o_ | | | | |
| | Veillonellales\|f_ | | | | |
| | Veillonellaceae\|g_ | | | | |
| | Megasphaera | | | | |
| marker36 | p_Firmicutes\|c_ | oral | 4.018755 | 0.001158 | 0.001158 |
| | Clostridia\|o_ | | | | |
| | Eubacteriales\|f_ | | | | |
| | Lachnospiraceae\|g_ | | | | |
| | Lachnospiraceae_ | | | | |
| | unclassified | | | | |
| marker37 | p_Firmicutes\|c_ | oral | 3.976852 | 0.029011 | 0.029011 |
| | Clostridia\|o_ | | | | |
| | Eubacteriales\|f_ | | | | |
| | Eubacteriales_ | | | | |
| | unclassified | | | | |
| marker38 | p_Firmicutes\|c_ | oral | 3.856901 | 0.01786 | 0.01786 |
| | Clostridia\|o_ | | | | |
| | Eubacteriales\|f_ | | | | |
| | Lachnospiraceae\|g_ | | | | |
| | Blautia\|s_ | | | | |
| | Blautia_ | | | | |
| | glucerasea | | | | |
| marker39 | p_Firmicutes\|c_ | oral | 3.810978 | 0.021537 | 0.021537 |
| | Clostridia\|o_ | | | | |
| | Eubacteriales\|f_ | | | | |
| | Lachnospiraceae\|g_ | | | | |
| | Fusicatenibacter | | | | |
| marker40 | p_Firmicutes\|c_ | oral | 3.81085 | 0.021537 | 0.021537 |
| | Clostridia\|o_ | | | | |
| | Eubacteriales\|f_ | | | | |
| | Lachnospiraceae\|g_ | | | | |
| | Fusicatenibacter\|s_ | | | | |
| | Fusicatenibacter_ | | | | |
| | saccharivorans | | | | |
| marker41 | p_Firmicutes\|c_ | oral | 3.732893 | 0.039554 | 0.039554 |
| | Clostridia\|o_ | | | | |
| | Eubacteriales\|f_ | | | | |
| | Lachnospiraceae\|g_ | | | | |
| | Blautia\|s_ | | | | |
| | Blautia_ | | | | |
| | massiliensis | | | | |
| marker42 | p_Firmicutes\|c_ | oral | 3.666498 | 0.009793 | 0.009793 |
| | Clostridia\|o_ | | | | |
| | Eubacteriales\|f_ | | | | |
| | Lachnospiraceae\|g_ | | | | |
| | Faecalicatena | | | | |
| marker43 | p_Firmicutes\|c_ | oral | 3.635945 | 0.039554 | 0.039554 |
| | Clostridia\|o_ | | | | |
| | Eubacteriales\|f_ | | | | |
| | Eubacteriales_ | | | | |
| | unclassified\|g_ | | | | |
| | Gemmiger\|s_ | | | | |
| | Gemmiger_ | | | | |
| | formicilis | | | | |
| marker44 | p_Firmicutes\|c_ | oral | 3.621163 | 0.011503 | 0.011503 |
| | Clostridia\|o_ | | | | |
| | Eubacteriales\|f_ | | | | |
| | Oscillospiraceae\|g_ | | | | |
| | Flavonifractor\|s_ | | | | |
| | Flavonifractor_ | | | | |
| | plautii | | | | |
| marker45 | p_Firmicutes\|c_ | oral | 3.620854 | 0.011503 | 0.011503 |
| | Clostridia\|o_ | | | | |
| | Eubacteriales\|f_ | | | | |
| | Oscillospiraceae\|g_ | | | | |
| | Flavonifractor | | | | |
| marker46 | p_ | oral | 3.532036 | 0.044291 | 0.044291 |
| | Proteobacteria\|c_ | | | | |
| | Betaproteo- | | | | |
| | bacteria\|o_ | | | | |
| | Burkholderiales\|f_ | | | | |
| | Sutterellaceae | | | | |
| marker47 | p_Firmicutes\|c_ | oral | 3.497744 | 0.00309 | 0.00309 |
| | Clostridia\|o_ | | | | |
| | Eubacteriales\|f_ | | | | |
| | Oscillospiraceae\|g_ | | | | |
| | Agathobaculum\|s_ | | | | |
| | Agathobaculum_ | | | | |
| | butyriciproducens | | | | |
| marker48 | p_Firmicutes\|c_ | oral | 3.48827 | 0.00309 | 0.00309 |
| | Clostridia\|o_ | | | | |
| | Eubacteriales\|f_ | | | | |
| | Oscillospiraceae\|g_ | | | | |
| | Agathobaculum | | | | |
| marker49 | p_Firmicutes\|c_ | oral | 3.468927 | 0.00309 | 0.00309 |
| | Negativicutes\|o_ | | | | |
| | Veillonellales\|f_ | | | | |
| | Veillonellaceae\|g_ | | | | |
| | Dialister\|s_ | | | | |
| | Dialister_ | | | | |
| | invisus | | | | |
| marker50 | p_Firmicutes\|c_ | oral | 3.465513 | 0.00309 | 0.00309 |
| | Negativicutes\|o_ | | | | |
| | Veillonellales\|f_ | | | | |
| | Veillonellaceae\|g_ | | | | |
| | Dialister | | | | |
| marker51 | p_Firmicutes\|c_ | oral | 3.324987 | 0.018953 | 0.018953 |
| | Negativicutes\|o_ | | | | |
| | Veillonellales\|f_ | | | | |
| | Veillonellaceae\|g_ | | | | |
| | Megasphaera\|s_ | | | | |
| | Megasphaera_sp_ | | | | |
| | NM10 | | | | |
| marker52 | p_Firmicutes\|c_ | oral | 3.324124 | 0.00309 | 0.00309 |
| | Bacilli\|o_ | | | | |
| | Lactobacillales\|f_ | | | | |
| | Strepto- | | | | |
| | coccaceae\|g_ | | | | |
| | Strepto- | | | | |
| | coccus\|s_ | | | | |
| | Strepto- | | | | |
| | coccus_ | | | | |
| | gordonii | | | | |
| marker53 | p_Proteobacteria\| | oral | 3.305614 | 0.025538 | 0.025538 |
| | c_Beta- | | | | |
| | proteobacteria\|o_ | | | | |
| | Burkholderiales\|f_ | | | | |
| | Sutterellaceae\|g_ | | | | |
| | Parasutterella | | | | |
| marker54 | p_Firmicutes\|c_ | oral | 3.291086 | 0.030863 | 0.030863 |
| | Clostridia\|o_ | | | | |
| | Eubacteriales\|f_ | | | | |
| | Lachnospiraceae\|g_ | | | | |
| | Lachnospiraceae_ | | | | |
| | unclassified\|s_ | | | | |
| | Lachnospiraceae_ | | | | |
| | bacterium_ | | | | |
| | NSJ_29 | | | | |
| marker55 | p_Proteobacteria\| | oral | 3.283363 | 0.025538 | 0.025538 |
| | c_Betaproteo- | | | | |
| | bacteria\|o_ | | | | |
| | Burkholderiales\|f_ | | | | |
| | Sutterellaceae\|g_ | | | | |
| | Parasutterellas_ | | | | |
| | Parasutterella_ | | | | |
| | excrementiho- | | | | |
| | minis | | | | |
| marker56 | p_Firmicutes\|c_ | oral | 3.270946 | 0.018953 | 0.018953 |
| | Clostridia\|o_ | | | | |
| | Eubacteriales\|f_ | | | | |
| | Lachnospiraceae\|g_ | | | | |
| | Enterocloster\|s_ | | | | |
| | Enterocloster_ | | | | |
| | asparagiformis | | | | |
| marker57 | p_ | oral | 3.270561 | 0.018863 | 0.018863 |
| | Proteobacteria\| | | | | |
| | c_Betaproteo- | | | | |
| | bacteria\|o_ | | | | |
| | Burkholderiales\|f_ | | | | |
| | Sutterellaceae\|g_ | | | | |
| | Sutterella\| | | | | |
| | s_Sutterella_ | | | | |
| | wadsworthensis | | | | |
| marker58 | p_Proteobacteria\| | oral | 3.269506 | 0.018863 | 0.018863 |
| | c_Betaproteo- | | | | |
| | bacteria\|o_ | | | | |
| | Burkholderiales\|f_ | | | | |
| | Sutterellaceae\|g_ | | | | |
| | Sutterella | | | | |
| marker59 | p_Firmicutes\|c_ | oral | 3.187874 | 0.00309 | 0.00309 |
| | Bacilli\|o_ | | | | |
| | Lactobacillales\|f_ | | | | |
| | Strepto- | | | | |
| | coccaceae\|g_ | | | | |
| | Streptococcus\|s_ | | | | |
| | Streptococcus_ | | | | |
| | oralis | | | | |
| marker60 | p_Firmicutes\|c_ | oral | 3.125641 | 0.018953 | 0.018953 |
| | Negativicutes\|o_ | | | | |
| | Selenomona- | | | | |
| | dales\|f_ | | | | |
| | Seleno- | | | | |
| | monadaceae\|g_ | | | | |
| | GGB79734_f_ | | | | |
| | Seleno- | | | | |
| | monadaceae\|s_ | | | | |
| | SGB15291_g_ | | | | |
| | GGB79734_ | | | | |
| | f_Seleno- | | | | |
| | monadaceae | | | | |
| marker61 | p_Firmicutes\|c_ | oral | 3.111429 | 0.018953 | 0.018953 |
| | Negativicutes\|o_ | | | | |
| | Selenomona- | | | | |
| | dales\|f_Seleno- | | | | |
| | monadaceae\|g_ | | | | |
| | GGB79734_ | | | | |
| | f_Seleno- | | | | |
| | monadaceae | | | | |
| marker62 | p_Firmicutes\|c_ | oral | 3.022376 | 0.009793 | 0.009793 |
| | Clostridia\|o_ | | | | |
| | Eubacteriales\|f_ | | | | |
| | Lachnospiraceae\|g_ | | | | |
| | Faecalicatena\|s_ | | | | |
| | Faecalicatena_ | | | | |
| | contorta | | | | |
| marker63 | p_Firmicutes\|c_ | tube | 5.505835 | 0.027184 | 0.027184 |
| | Bacilli\|o_ | feeding | | | |
| | Lactobacillales\|f_ | | | | |
| | Enterococcaceae | | | | |
| marker64 | p_Firmicutes\|c_ | tube | 5.494695 | 0.034475 | 0.034475 |
| | Bacilli\|o_ | feeding | | | |
| | Lactobacillales\|f_ | | | | |
| | Enterococcaceae\|g_ | | | | |
| | Enterococcus | | | | |

TABLE 8
Differental abundance of Eukaryotes (fungi) in
non-mortality and mortality cohort

| | | enrich_ | | | |
|---|---|---|---|---|---|
| | feature | group | ef_lda | pvalue | padj |
| marker1 | p_Ascomycota\|c_ | mor- | 5.396554 | 0.046945 | 0.046945 |
| | Saccharomycetes\|o_ | tality | | | |
| | Saccharo- | | | | |
| | mycetales\|f_ | | | | |
| | Saccharomycesceae | | | | |
| marker2 | p_Ascomycota\|c_ | non- | 5.077177 | 0.015117 | 0.015117 |
| | Saccharomycetes\|o_ | mor- | | | |
| | Saccharo- | tality | | | |
| | mycetales\|f_ | | | | |
| | Saccharomycesceae\| | | | | |
| | g_Saccharomyces | | | | |
| marker3 | p_Ascomycota\|c_ | non- | 4.60785 | 0.01496 | 0.01496 |
| | Eurotiomycetes\|o_ | mor- | | | |
| | Eurotiales\|f_ | tality | | | |
| | Aspergillaceae | | | | |
| marker4 | p_Ascomycota\|c_ | non- | 4.591805 | 0.026484 | 0.026484 |
| | Saccharomycetes\|o_ | mor- | | | |
| | Saccharo- | tality | | | |
| | mycetales\|f_ | | | | |
| | Saccharomycesceae\| | | | | |
| | g_Eremothecium | | | | |
| marker5 | p_Ascomycota\|c_ | non- | 4.575471 | 0.008833 | 0.008833 |
| | Eurotiomycetes\|o_ | mor- | | | |
| | Eurotiales\|f_ | tality | | | |
| | Aspergillaceae\|g_ | | | | |
| | Aspergillus | | | | |
| marker6 | p_Ascomycota\|c_ | non- | 4.152092 | 0.019035 | 0.019035 |
| | Saccharomycetes\|o_ | mor- | | | |
| | Saccharo- | tality | | | |
| | mycetales\|f_ | | | | |
| | Pichiaceae | | | | |
| marker7 | p_Ascomycota\|c_ | non- | 4.142815 | 0.011923 | 0.011923 |
| | Sordariomycetes\|o_ | mor- | | | |
| | Hypocreales\|f_ | tality | | | |
| | Nectriaceae\|g_ | | | | |
| | Fusarium\|s_ | | | | |
| | Fusarium_musae | | | | |
| marker8 | p_Ascomycota\|c_ | non- | 4.105453 | 0.022887 | 0.022887 |
| | Eurotiomycetes\|o_ | mor- | | | |
| | Eurotiales\|f_ | tality | | | |
| | Aspergillaceae\|g_ | | | | |
| | Aspergillus\|s_ | | | | |
| | Aspergillus_ | | | | |
| | luchuensis | | | | |
| marker9 | p_Ascomycota\|c_ | non- | 4.103405 | 0.038579 | 0.038579 |
| | Sordariomycetes\|o_ | mor- | | | |
| | Magnaporthales\|f_ | tality | | | |
| | Pyriculariaceae\|g_ | | | | |
| | Pyricularials_ | | | | |
| | Pyricularia_ | | | | |
| | pennisetigena | | | | |
| marker10 | p_Ascomycota\|c_ | non- | 3.993562 | 0.00948 | 0.00948 |
| | Saccharomycetes\|o_ | mor- | | | |
| | Saccharo- | tality | | | |
| | mycetales\|f_ | | | | |
| | Saccharo- | | | | |
| | mycesaceae\| | | | | |
| | g_Naumovozyma\|s_ | | | | |
| | Naumovozyma_ | | | | |
| | castellii | | | | |
| marker11 | p_Ascomycota\|c_ | non- | 3.928006 | 0.02881 | 0.02881 |
| | Sordariomycetes\|o_ | mor- | | | |
| | Hypocreales\|f_ | tality | | | |
| | Clavicipitaceae | | | | |
| marker12 | p_Ascomycota\|c_ | non- | 3.874608 | 0.011056 | 0.011056 |
| | Saccharomycetes\|o_ | mor- | | | |
| | Saccharomycetales\|f_ | tality | | | |
| | Pichiaceae\|g_ | | | | |
| | Ogataea\|s_ | | | | |
| | Ogataea_ | | | | |
| | parapolymorpha | | | | |
| marker13 | p_Ascomycota\|c_ | non- | 3.874374 | 0.011056 | 0.011056 |
| | Saccharomycetes\|o_ | mor- | | | |
| | Saccharo- | tality | | | |
| | mycetales\|f_ | | | | |
| | Pichiaceae\|g_ | | | | |
| | Ogataea | | | | |
| marker14 | p_Ascomycota\|c_ | non- | 3.86723 | 0.02881 | 0.02881 |
| | Saccharomycetes\|o_ | mor- | | | |
| | Saccharo- | tality | | | |
| | mycetales\|f_ | | | | |
| | Saccharo- | | | | |
| | mycesaceae\| | | | | |
| | g_Saccharo- | | | | |
| | myces\|s_ | | | | |
| | Saccharomyces_ | | | | |
| | mikatae | | | | |
| marker15 | p_Ascomycota\|c_ | non- | 3.866172 | 0.005387 | 0.005387 |
| | Saccharomycetes\|o_ | mor- | | | |
| | Saccharo- | tality | | | |
| | mycetales\|f_ | | | | |
| | Dipodascaceae | | | | |
| marker16 | p_Ascomycota\|c_ | non- | 3.862903 | 0.005387 | 0.005387 |
| | Saccharomycetes\|o_ | mor- | | | |
| | Saccharo- | tality | | | |
| | mycetales\|f_ | | | | |
| | Dipodascaceae\|g_ | | | | |
| | Yarrowia | | | | |
| marker17 | p_Ascomycota\|c_ | non- | 3.861844 | 0.005387 | 0.005387 |
| | Saccharomycetes\|o_ | mor- | | | |
| | Saccharo- | tality | | | |
| | mycetales\|f_ | | | | |
| | Dipodascaceae\|g_ | | | | |
| | Yarrowials_ | | | | |
| | Yarrowia_lipolytica | | | | |
| marker18 | p_Ascomycota\|c_ | non- | 3.856281 | 0.029084 | 0.029084 |
| | Eurotiomycetes\|o_ | mor- | | | |
| | Eurotiales\|f_ | tality | | | |
| | Aspergillaceae\|g_ | | | | |
| | Aspergillus\|s_ | | | | |
| | Aspergillus_ | | | | |
| | flavus | | | | |
| marker19 | p_Ascomycota\|c_ | non- | 3.821403 | 0.020355 | 0.020355 |
| | Saccharomycetes\|o_ | mor- | | | |
| | Saccharo- | tality | | | |
| | mycetales\|f_ | | | | |
| | Pichiaceae\|g_ | | | | |
| | Brettanomyces | | | | |
| marker20 | p_Ascomycota\|c_ | non- | 3.778144 | 0.011056 | 0.011056 |
| | Saccharomycetes\|o_ | mor- | | | |
| | Saccharo- | tality | | | |
| | mycetales\|f_ | | | | |
| | Pichiaceae\|g_ | | | | |
| | Brettanomyces\|s_ | | | | |
| | Brettanomyces_ | | | | |
| | bruxellensis | | | | |
| marker21 | p_Ascomycota\|c_ | non- | 3.77598 | 0.020355 | 0.020355 |
| | Saccharomycetes\|o_ | mor- | | | |
| | Saccharo- | tality | | | |
| | mycetales\|f_ | | | | |
| | Saccharo- | | | | |
| | mycesceae\|g_ | | | | |
| | Saccharomyces\|s_ | | | | |
| | Saccharomyces_ | | | | |
| | kudriavzevii | | | | |
| marker22 | p_Basidiomycota\|c_ | non- | 3.768505 | 0.018053 | 0.018053 |
| | Agaricomycetes\|o_ | mor- | | | |
| | Agaricales\|f_ | tality | | | |
| | Marasmiaceae | | | | |
| marker23 | p_Basidiomycota\|c_ | non- | 3.768505 | 0.018053 | 0.018053 |
| | Agaricomycetes\|o_ | mor- | | | |
| | Agaricales\|f_ | tality | | | |
| | Marasmiaceae\|g_ | | | | |
| | Marasmius | | | | |
| marker24 | p_Basidiomycota\|c_ | non- | 3.768505 | 0.018053 | 0.018053 |
| | Agaricomycetes\|o_ | mor- | | | |
| | Agaricales\|f_ | tality | | | |
| | Marasmiaceae\|g_ | | | | |
| | Marasmius\|s_ | | | | |
| | Marasmius_ | | | | |
| | oreades | | | | |

TABLE 9
Differential abundance of Prokaryotes (bacteria) in
non mortality and mortality cohort

| | | enrich_ | | | |
|---|---|---|---|---|---|
| | feature | group | ef_lda | pvalue | padj |
| marker1 | p_Firmicutes\|c_ | mortality | 5.42941 | 0.018466 | 0.018466 |
| | Bacilli\|o_ | | | | |
| | Lactobacillales\|f_ | | | | |
| | Enterococcaceae\|g_ | | | | |
| | Enterococcus | | | | |
| marker2 | p_Firmicutes\|c_ | mortality | 4.76958 | 0.031073 | 0.031073 |
| | Bacilli\|o_ | | | | |
| | Lactobacillales\|f_ | | | | |
| | Enterococcaceae\|g_ | | | | |
| | Enterococcus\|s_ | | | | |
| | Enterococcus_ | | | | |
| | faecalis | | | | |
| marker3 | p_Firmicutes\|c_ | non- | 4.502769 | 0.002295 | 0.002295 |
| | Bacilli\|o_ | mortality | | | |
| | Bacillales | | | | |
| marker4 | p_Firmicutes\|c_ | non- | 4.50215 | 0.003109 | 0.003109 |
| | Bacilli\|o_ | mortality | | | |
| | Bacillales\|f_ | | | | |
| | Staphylococcaceae | | | | |
| marker5 | p_Firmicutes\|c_ | non- | 4.498894 | 0.011923 | 0.011923 |
| | Bacilli\|o_ | mortality | | | |
| | Bacillales\|f_ | | | | |
| | Staphylo- | | | | |
| | coccaceae\|g_ | | | | |
| | Staphylococcus | | | | |
| marker6 | p_Firmicutes\|c_ | non- | 4.472746 | 0.036007 | 0.036007 |
| | Clostridia\|o_ | mortality | | | |
| | Eubacteriales\|f_ | | | | |
| | Clostridiaceae | | | | |
| marker7 | p_Firmicutes\|c_ | non- | 4.189428 | 0.032534 | 0.032534 |
| | Bacilli\|o_ | mortality | | | |
| | Lactobacillales\|f_ | | | | |
| | Streptococcaceae | | | | |
| marker8 | p_Firmicutes\|c_ | non- | 4.18536 | 0.02583 | 0.02583 |
| | Bacillilo_ | mortality | | | |
| | Lactobacillales\|f_ | | | | |
| | Streptoco- | | | | |
| | ccaceae\|g | | | | |
| | Streptococcus | | | | |
| marker9 | p_Firmicutes\|c_ | non- | 3.754465 | 0.004514 | 0.004514 |
| | Clostridia\|o_ | mortality | | | |
| | Eubacteriales\|f_ | | | | |
| | Lachnospiraceae\|g_ | | | | |
| | Blautia\|s_ | | | | |
| | Blautia_obeum | | | | |
| marker10 | p_Firmicutes\|c_ | non- | 3.729701 | 0.011923 | 0.011923 |
| | Clostridia\|o_ | mortality | | | |
| | Eubacteriales\|f_ | | | | |
| | Lachnospiraceae\|g_ | | | | |
| | Dorea | | | | |
| marker11 | p_Firmicutes\|c_ | non- | 3.639663 | 0.011923 | 0.011923 |
| | Clostridia\|o_ | mortality | | | |
| | Eubacteriales\|f_ | | | | |
| | Lachnospiraceae\|g_ | | | | |
| | Dorea\|s_Dorea_ | | | | |
| | longicatena | | | | |
| marker12 | p_Actino- | non- | 3.482993 | 0.029084 | 0.029084 |
| | bacteria\|c_ | mortality | | | |
| | Actinomycetia\|o_ | | | | |
| | Bifido- | | | | |
| | bacteriales\|f_ | | | | |
| | Bifido- | | | | |
| | bacteriaceae\|g_ | | | | |
| | Bifidobacterium\|s_ | | | | |
| | Bifidobacterium_ | | | | |
| | dentium | | | | |
| marker13 | p_Firmicutes\|c_ | non- | 3.446189 | 0.029084 | 0.029084 |
| | Clostridia\|o_ | mortality | | | |
| | Eubacteriales\|f_ | | | | |
| | Lachnospiraceae\|g_ | | | | |
| | Coprococcus | | | | |
| marker14 | p_Firmicutes\|c_ | non- | 3.225344 | 0.029084 | 0.029084 |
| | CFGB2982_p | mortality | | | |
| | Firmicutes\|o_ | | | | |
| | OFGB2982_c_ | | | | |
| | CFGB2982_p_ | | | | |
| | Firmicutes | | | | |
| marker15 | p_Firmicutes\|c_ | non- | 3.225343 | 0.029084 | 0.029084 |
| | CFGB2982_p_ | mortality | | | |
| | Firmicutes\|o_ | | | | |
| | OFGB2982_c_ | | | | |
| | CFGB2982_p_ | | | | |
| | Firmicutes\|f_ | | | | |
| | FGB2982_c_ | | | | |
| | CFGB2982_p_ | | | | |
| | Firmicutes\|g_ | | | | |
| | GGB9342_f_ | | | | |
| | FGB2982_c_ | | | | |
| | cFGB2982_p_ | | | | |
| | Firmicutes | | | | |
| marker16 | p_Firmicutes\|c_ | non- | 3.225343 | 0.029084 | 0.029084 |
| | CFGB2982_p_ | mortality | | | |
| | Firmicutes | | | | |
| marker17 | p_Firmicutes\|c_ | non- | 3.225343 | 0.029084 | 0.029084 |
| | CFGB2982_p_ | mortality | | | |
| | Firmicutes\|o_ | | | | |
| | OFGB2982_c_ | | | | |
| | CFGB2982_p_ | | | | |
| | Firmicutes\|f_ | | | | |
| | FGB2982_c_ | | | | |
| | CFGB2982_ | | | | |
| | p_Firmicutes\|g_ | | | | |
| | GGB9342_f | | | | |
| | FGB2982_c_ | | | | |
| | cFGB2982_p_ | | | | |
| | Firmicutess_ | | | | |
| | SGB14306_g_ | | | | |
| | GGB9342_ | | | | |
| | f_FGB2982_c_ | | | | |
| | CFGB2982_ | | | | |
| | p_Firmicutes | | | | |
| marker18 | p_Firmicutes\|c_ | non- | 3.225343 | 0.029084 | 0.029084 |
| | CFGB2982_p_ | mortality | | | |
| | Firmicutes\|o_ | | | | |
| | OFGB2982_c_ | | | | |
| | CFGB2982_p_ | | | | |
| | Firmicutes\|f_ | | | | |
| | FGB2982_c_ | | | | |
| | CFGB2982_p_ | | | | |
| | Firmicutes | | | | |
| marker19 | p_Firmicutes\|c_ | non- | 3.203528 | 0.011923 | 0.011923 |
| | Clostridia\|o_ | mortality | | | |
| | Eubacteriales\|f_ | | | | |
| | Oscillospiraceae\|g_ | | | | |
| | GGB9699_f_ | | | | |
| | Oscillospiraceae | | | | |
| marker20 | p_Firmicutes\|c_ | non- | 3.203527 | 0.011923 | 0.011923 |
| | Clostridia\|o_ | mortality | | | |
| | Eubacteriales\|f_ | | | | |
| | Oscillospiraceae\|g_ | | | | |
| | GGB9699_f_ | | | | |
| | Oscillospiraceae\|s_ | | | | |
| | SGB15216_g_ | | | | |
| | GGB9699_f_ | | | | |
| | Oscillospiraceae | | | | |
| marker21 | p_Actino- | non- | 3.097793 | 0.029084 | 0.029084 |
| | bacteria\|c_ | mortality | | | |
| | Coriobacteriia\|o_ | | | | |
| | Coriobacteriales\|f_ | | | | |
| | Atopobiaceae | | | | |

### Discussion of Examples 1-7

In this cohort study, significant differences in microbiome structure were demonstrated between subjects with SE and chronic epilepsy. It was shown that over the course of SE resolution and hospitalization, microbiome diversity changed differently in those with SE of known cause and those with NORSE. Specific microbiota taxa were then shown to correlate with cytokines known to be upregulated in NORSE and SE, providing insight into inflammation as one possible mechanism through which microbiome changes may impact neurologic injury in SE. Lastly, it was demonstrated that oral feeding and specific microbiome profiles predict survival in SE, further supporting a role for the gut microbiome in impacting disease course and neurologic injury in SE.

This study was the first to examine gut microbiome in humans with SE. Recent experimental models of SE-induced epilepsy have demonstrated gut microbiota changes after SE (PMID: 38485093), and improvement in SE duration after treatment with rifaximin, which promotes beneficial gut microbiota (PMID: 38608741). The impact of the ketogenic diet on SE and epilepsy (PMID: 33155184) has long been recognized and is mediated by gut microbiome (PMID: 29804833).

Reports describing the effectiveness of anakinra (Dilena et al., 2019; Kenney-Jung et al., 2016) and tocilizumab (Aledo-Serrano et al., 2022; Girardin et al., 2023; Jun et al., 2018) suggested that targeting IL-1b and IL-6 cytokine pathways could be therapeutically effective. However, such immunotherapies come with risks of immunosuppression, which are amplified in a critical illness setting. It was found that cytokine upregulation correlated with specific gut microbiota. E. bolteae and Akkermansia muciniphila correlated with cytokine upregulation including IL-1b and IL-6, in the NORSE cohort and was significantly enriched during SE in the NORSE cohort. Akkermansia muciniphila has previously been shown to be upregulated in other neurologic conditions associated with inflammation, including multiple sclerosis (PMID: 36113426), and can modulate CD4 T cells (PMID: 28893978). Other prokaryotic and eukaryotic species (e.g. Enterobacter hormaechei, C. dubliniensis) correlated with cytokines, and have been described in critically ill patients (PMID: 38385742), so may be related to the immunocompromised conditions many of the SE and NORSE subjects were under.

### REFERENCES

1. Aledo-Serrano, A., Hariramani, R., Gonzalez-Martinez, A., Alvarez-Troncoso, J., Toledano, R., Bayat, A., Garcia-Morales, I., Becerra, J. L., Villegas-Martinez, I., Beltran-Corbellini, A., & Gil-Nagel, A. (2022). Anakinra and tocilizumab in the chronic phase of febrile infection-related epilepsy syndrome (FIRES): Effectiveness and safety from a case-series. Seizure, 100, 51-55. https://doi.org/10.1016/j.seizure.2022.06.012
2. Beghini, F., McIver, L. J., Blanco-Miguez, A., Dubois, L., Asnicar, F., Maharjan, S., Mailyan, A., Manghi, P., Scholz, M., Thomas, A. M., Valles-Colomer, M., Weingart, G., Zhang, Y., Zolfo, M., Huttenhower, C., Franzosa, E. A., & Segata, N. (2021). Integrating taxonomic, functional, and strain-level profiling of diverse microbial communities with bioBakery 3. eLife, 10, e65088. https://doi.org/10.7554/eLife.65088
3. Betjemann, J. P., & Lowenstein, D. H. (2015). Status epilepticus in adults. Lancet Neurol, 14(6), 615-624. https://doi.org/10.1016/s1474-4422(15)00042-3
4. Blanco-Miguez, A., Beghini, F., Cumbo, F., McIver, L. J., Thompson, K. N., Zolfo, M., Manghi, P., Dubois, L., Huang, K. D., Thomas, A. M., Nickols, W. A., Piccinno, G., Piperni, E., Punčochář, M., Valles-Colomer, M., Tett, A., Giordano, F., Davies, R., Wolf, J., . . . Segata, N. (2023). Extending and improving metagenomic taxonomic profiling with uncharacterized species using MetaPhlAn 4. Nature Biotechnology, 41(11), 1633-1644. https://doi.org/10.1038/s41587-023-01688-w
5. Bolger, A. M., Lohse, M., & Usadel, B. (2014). Trimmomatic: a flexible trimmer for Illumina sequence data. Bioinformatics, 30(15), 2114-2120. https://doi.org/10.1093/bioinformatics/btul70
6. Dilena, R., Mauri, E., Aronica, E., Bernasconi, P., Bana, C., Cappelletti, C., Carrabba, G., Ferrero, S., Giorda, R., Guez, S., Scalia Catenacci, S., Triulzi, F., Barbieri, S., Calderini, E., & Vezzani, A. (2019). Therapeutic effect of Anakinra in the relapsing chronic phase of febrile infection-related epilepsy syndrome. Epilepsia Open, 4(2), 344-350. https://doi.org/10.1002/epi4.12317
7. Gaspard, N., Foreman, B. P., Alvarez, V., Cabrera Kang, C., Probasco, J. C., Jongeling, A. C., Meyers, E., Espinera, A., Haas, K. F., Schmitt, S. E., Gerard, E. E., Gofton, T., Kaplan, P. W., Lee, J. W., Legros, B., Szaflarski, J. P., Westover, B. M., LaRoche, S. M., & Hirsch, L. J. (2015). New-onset refractory status epilepticus: Etiology, clinical features, and outcome. Neurology, 85(18), 1604-1613. https://doi.org/10.1212/wnl.0000000000001940
8. Girardin, M. L., Flamand, T., Roignot, O., Abi Warde, M. T., Mutschler, V., Voulleminot, P., Guillot, M., Dinkelacker, V., & De Saint-Martin, A. (2023). Treatment of new onset refractory status epilepticus/febrile infection-related epilepsy syndrome with tocilizumab in a child and a young adult. Epilepsia, 64(6), e87-e92. https://doi.org/10.1111/epi.17591
9. Hanin, A., Cespedes, J., Dorgham, K., Pulluru, Y., Gopaul, M., Gorochov, G., Hafler, D. A., Navarro, V., Gaspard, N., & Hirsch, L. J. (2023). Cytokines in New-Onset Refractory Status Epilepticus Predict Outcomes. Ann Neurol, 94(1), 75-90. https://doi.org/10.1002/ana.26627
10. Hanin, A., Cespedes, J., Huttner, A., Strelnikov, D., Gopaul, M., DiStasio, M., Vezzani, A., Hirsch, L. J., & Aronica, E. (2023). Neuropathology of New-Onset Refractory Status Epilepticus (NORSE). J Neurol, 270(8), 3688-3702. https://doi.org/10.1007/s00415-023-11726-x
11. Hirsch, L. J., Gaspard, N., van Baalen, A., Nabbout, R., Demeret, S., Loddenkemper, T., Navarro, V., Specchio, N., Lagae, L., Rossetti, A. O., Hocker, S., Gofton, T. E., Abend, N. S., Gilmore, E. J., Hahn, C., Khosravani, H., Rosenow, F., & Trinka, E. (2018). Proposed consensus definitions for new-onset refractory status epilepticus (NORSE), febrile infection-related epilepsy syndrome (FIRES), and related conditions. Epilepsia, 59(4), 739-744. https://doi.org/10.1111/epi.14016
12. Jun, J. S., Lee, S. T., Kim, R., Chu, K., & Lee, S. K. (2018). Tocilizumab treatment for new onset refractory status epilepticus. Ann Neurol, 84(6), 940-945. https://doi.org/10.1002/ana.25374
13. Kamada, N., Seo, S. U., Chen, G. Y., & Nunez, G. (2013). Role of the gut microbiota in immunity and inflammatory disease. Nat Rev Immunol, 13(5), 321-335. https://doi.org/10.1038/nri3430
14. Kenney-Jung, D. L., Vezzani, A., Kahoud, R. J., LaFrance-Corey, R. G., Ho, M. L., Muskardin, T. W., Wirrell, E. C., Howe, C. L., & Payne, E. T. (2016). Febrile infection-related epilepsy syndrome treated with anakinra. Ann Neurol, 80(6), 939-945. https://doi.org/10.1002/ana.24806
15. Kwack, D. W., & Kim, D. W. (2022). The Increased Interleukin-6 Levels Can Be an Early Diagnostic Marker for New-Onset Refractory Status Epilepticus. J Epilepsy Res, 12(2), 78-81. https://doi.org/10.14581/jer.22015
16. Langmead, B., & Salzberg, S. L. (2012). Fast gapped-read alignment with Bowtie 2. Nature Methods, 9(4), 357-359. https://doi.org/10.1038/nmeth.1923
17. Langmead, B., Wilks, C., Antonescu, V., & Charles, R. (2018). Scaling read aligners to hundreds of threads on general-purpose processors. Bioinformatics, 35(3), 421-432. https://doi.org/10.1093/bioinformatics/bty648
18. Lu, J., Breitwieser, F. P., Thielen, P., & Salzberg, S. L. (2017). Bracken: estimating species abundance in metagenomics data. PeerJ Computer Science, 3, e104. https://doi.org/10.7717/peerj-cs.104
19. McMurdie, P. J., & Holmes, S. (2013). phyloseq: An R Package for Reproducible Interactive Analysis and Graphics of Microbiome Census Data. PLOS ONE, 8(4), e61217. https://doi.org/10.1371/journal.pone.0061217
20. Ritter, L. M., & Nashef, L. (2021). New-onset refractory status epilepticus (NORSE). Practical Neurology, 21(2), 119-127. https://doi.org/10.1136/practneurol-2020-002534
21. Rossetti, A. O., & Lowenstein, D. H. (2011). Management of refractory status epilepticus in adults: still more questions than answers. Lancet Neurol, 10(10), 922-930. https://doi.org/10.1016/s1474-4422(11)70187-9
22. Sakuma, H., Tanuma, N., Kuki, I., Takahashi, Y., Shiomi, M., & Hayashi, M. (2015). Intrathecal overproduction of proinflammatory cytokines and chemokines in febrile infection-related refractory status epilepticus. J Neurol Neurosurg Psychiatry, 86(7), 820-822. https://doi.org/10.1136/jnnp-2014-309388
23. Schirmer, M., Smeekens, S. P., Vlamakis, H., Jaeger, M., Oosting, M., Franzosa, E. A., Ter Horst, R., Jansen, T., Jacobs, L., Bonder, M. J., Kurilshikov, A., Fu, J., Joosten, L. A. B., Zhernakova, A., Huttenhower, C., Wijmenga, C., Netea, M. G., & Xavier, R. J. (2016). Linking the Human Gut Microbiome to Inflammatory Cytokine Production Capacity. Cell, 167(4), 1125-1136.e1128. https://doi.org/10.1016/j.cell.2016.10.020
24. Sculier, C., & Gaspard, N. (2019). New onset refractory status epilepticus (NORSE). Seizure, 68, 72-78. https://doi.org/https://doi.org/10.1016/j.seizure.2018.09.018
25. Shaw, M. H., Kamada, N., Kim, Y. G., & Nunez, G. (2012). Microbiota-induced IL-1beta, but not IL-6, is critical for the development of steady-state TH17 cells in the intestine. J Exp Med, 209(2), 251-258. https://doi.org/10.1084/jem.20111703
26. Sheikh, Z., & Hirsch, L. J. (2023). A practical approach to in-hospital management of new-onset refractory status epilepticus/febrile infection related epilepsy syndrome [Review]. Frontiers in Neurology, 14. https://doi.org/10.3389/fneur.2023.1150496
27. Shrestha, A., Wood, E. L., Berrios-Siervo, G., Stredny, C. M., Boyer, K., Vega, C., Nangia, S., Muscal, E., & Eschbach, K. (2023). Long-term neuropsychological outcomes in children with febrile infection-related epilepsy syndrome (FIRES) treated with anakinra. Front Neurol, 14, 1100551. https://doi.org/10.3389/fneur.2023.1100551
28. Singh, V., Roth, S., Llovera, G., Sadler, R., Garzetti, D., Stecher, B., Dichgans, M., & Liesz, A. (2016). Microbiota Dysbiosis Controls the Neuroinflammatory Response after Stroke. J Neurosci, 36(28), 7428-7440. https://doi.org/10.1523/jneurosci.1114-16.2016
29. Strzelczyk, A., Ansorge, S., Hapfelmeier, J., Bonthapally, V., Erder, M. H., & Rosenow, F. (2017). Costs, length of stay, and mortality of super-refractory status epilepticus: A population-based study from Germany. Epilepsia, 58(9), 1533-1541. https://doi.org/10.1111/epi.13837
30. Taraschenko, O., Pavuluri, S., Schmidt, C. M., Pulluru, Y. R., & Gupta, N. (2023). Seizure burden and neuropsychological outcomes of new-onset refractory status epilepticus: Systematic review. Front Neurol, 14, 1095061. https://doi.org/10.3389/fneur.2023.1095061
31. Tharmaraja, T., Ho, J. S. Y., Neligan, A., & Rajakulendran, S. (2023). The etiology and mortality of new-onset refractory status epilepticus (NORSE) in adults: A systematic review and meta-analysis. Epilepsia, 64(5), 1113-1124. https://doi.org/10.1111/epi.17523
32. Tremlett, H., Bauer, K. C., Appel-Cresswell, S., Finlay, B. B., & Waubant, E. (2017). The gut microbiome in human neurological disease: A review. Ann Neurol, 81(3), 369-382. https://doi.org/10.1002/ana.24901
33. Wood, D. E., Lu, J., & Langmead, B. (2019). Improved metagenomic analysis with Kraken 2. Genome Biology, 20(1), 257. https://doi.org/10.1186/s13059-019-1891-0
34. PMID: 38485093
35. PMID: 38608741
36. PMID: 33155184
37. PMID: 29804833
38. PMID: 36113426
39. PMID: 28893978
40. PMID: 38385742

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for the diagnosis of a subject suffering from Status Epilepticus (SE) or at risk of suffering from SE, said method comprising:
(a) determining in a fecal microbiota sample isolated from the subject the level of at least one strain of fungi from one or more class Saccharomycetales or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Enterococcus faecalis, Enterocloster bolteae, Escherichia coli, Sellimonas intestinalis, Akkermansia muciniphila, Fusicatenibacter saccharivorans, Lacticaseibacillus rhamnosus, Streptococcus anginosus, and Roseburia hominis or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in a control fecal microbiota, and
(c) identifying that the subject has SE or at risk of suffering from SE, wherein the level of at least one of the strains determined in step (a) is higher than in the control.

2. A method for the diagnosis of a subject suffering from Status Epilepticus (SE) or at risk of suffering from SE, said method comprising:
(a) determining in a fecal microbiota sample isolated from the subject the level of at least one strain of fungi from one or more species selected from Trichoderma breve and Fusarium verticilloides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Clostridium leptum, Bacteroides stercoris, Fusicatenibacter saccharivorans, and Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in a control fecal microbiota, and
(c) identifying that the subject has SE or at risk of suffering from SE, wherein the level of at least one of the strains determined in step (a) is lower than in the control.

3. A method for the diagnosis of a subject suffering from New Onset Refractory Status Epilepticus (NORSE) or at risk of suffering from NORSE, said method comprising:
(a) determining in a fecal microbiota sample isolated from the subject the level of at least one strain of fungi from genus Nakaseomyces glabratus and Marasmius oreades or closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Enterococcus faecalis, Enterococcus faecium, Escherichia coli, Enterocloster bolteae, Lacticaseibacillus paracasei, and an Clostridiaceae_bacterium_OM02_2AC species closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in a control fecal microbiota, and
(c) identifying that the subject is predisposed to NORSE or at risk of suffering from NORSE, wherein the level of at least one of the strains determined in step (a) is higher than in the control.

4. A method for the diagnosis of a subject suffering from New Onset Refractory Status Epilepticus (NORSE) or at risk of suffering from NORSE, said method comprising:
(a) determining in a fecal microbiota sample isolated from the subject the level of at least one strain of fungi from one or more species selected from Trichoderma breve and Fusarium verticilloides or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species from Eubacterium rectale or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in a control fecal microbiota, and
(c) identifying that the subject has NORSE or at risk of suffering from NORSE, wherein the level of at least one of the strains determined in step (a) is lower than in the control.

5.-7. (canceled)

8. A method for predicting risk of mortality associated with SE or NORSE a subject presenting with SE or NORSE, said method comprising:
(a) determining in a fecal microbiota sample isolated from the subject the level of at least one strain of fungi from one or more species selected from Family Saccharomycetaceae or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Enterococcaceae, Genus Enterococcus, and Species Enterococcus faecalis, or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in the control fecal microbiota, and
(c) identifying that the subject is at risk of mortality associated with SE or NORSE, wherein the level of at least one of the strains determined in step (a) is higher than in the control.

9. The method of claim 8, wherein the level of at least one of the strains determined in step (a) is at least 2-log-fold to 10-log-fold higher than in the control.

10. A method for predicting risk of mortality associated with SE or NORSE in a subject presenting with SE or NORSE, said method comprising:
(a) determining in a fecal microbiota sample isolated from the subject the level of at least one strain of fungi from one or more species selected from Family Aspergillaceae, Family Clavicipitaceae, Family Dipodascaceae, Family Marasmiaceae, Family Nectriaceae, Family Pichiaceae, Family Pyriculariaceae, Family Saccharomycetaceae, Genus Aspergillus, Genus Brettanomyces, Genus Eremothecium, Genus Fusarium, Genus Marasmius, Genus Naumovozyma, Genus Ogataea, Genus Pyricularia, Genus Saccharomyces, Genus Yarrowia, Species Aspergillus flavus, Species Aspergillus luchuensis, Species Brettanomyces bruxellensis, Species Fusarium musae, Species Marasmius oreades, Species Naumovozyma castelhii, Species Ogataea parapolymorpha, Species Pyricularia pennisetigena, Species Saccharomyces kudriavzevii, Species Saccharomyces mikatae, and Species Yarrowia lipolytica or a closely related OTU which has at least 90% (or at least 95%, or at least 97%, or at least 99%) sequence identity to 18S rRNA over its entire length or has at least 90% (or at least 95%, or at least 99%) sequence identity to any single or ITS1 or ITS2 region of the 18S rRNA and/or the level of at least one strain of bacteria from one or more species selected from Family Atopobiaceae, Family Bifidobacteriaceae, Family Clostridiaceae, Family FGB2982 c CFGB2982 p Firmicutes, Family Lachnospiraceae, Family Oscillospiraceae, Family Staphylococcaceae, Genus Bifidobacterium, Genus Blautia, Genus Coprococcus, Genus Dorea, Genus GGB9342 f FGB2982 c CFGB2982 p Firmicutes, Genus GGB9699 f Oscillospiraceae, Genus Staphylococcus, Genus Streptococcus, Species Bifidobacterium dentium, Species Blautia obeum, Species Dorea longicatena, Species SGB14306 g GGB9342 f FGB2982 c CFGB2982 p Firmicutes, and Species SGB15216 g GGB9699f Oscillospiraceae or a closely related OTU which has at least 90% (or at least 95%, or at least 97%, or at least 99%) sequence identity to 16S rRNA over its entire length or has at least 90% (or at least 95%, or at least 99%) sequence identity to any single V region of 16S rRNA, and
(b) comparing the level(s) determined in step (a) to the level(s) of the same fungi and/or bacteria in a control fecal microbiota, and
(c) identifying that the subject is at risk of mortality associated with SE or NORSE, wherein the level of at least one of the strains determined in step (a) is lower than in the control.

11. The method of claim 10, wherein the level of at least one of the strains determined in step (a) is at least 2-log-fold to 10-log-fold lower than in the control.

12. The method of claim 8, wherein the control fecal microbiota is fecal microbiota of subjects presenting with SE or NORSE that did not experience mortality.

13. The method of claim 8, wherein the level of fungi and/or bacteria is determined by a method selected from shotgun metagenomics, quantitative PCR (qPCR), high-throughput sequencing, transcriptomic analysis, bacterial or fungal genotype pattern based fingerprinting (DNA fingerprinting), inflammatory cytokine, metabolomics, bacterial or fungal gene profiling, and proteomic analysis.

14. The method of claim 8, wherein the fecal sample had been isolated from the subject during status epilepticus or within 3 days of status epilepticus resolution.

15. The method of claim 8, further comprising isolating the fecal sample from the subject prior to step (a).

16. The method of claim 8, wherein determining the level of at least one strain of fungi and/or bacteria comprises extracting DNA from bacterial and fungal species at the same time.

17. The method of claim 8, further comprising determining the level of one or more cytokines in a blood sample from the subject.

18. The method of claim 17, wherein the subject has SE or is likely to develop SE when the level of at least one of GCSF, IL10, IL12p70, IL1b, IL4, TNFa, and IL17A in the sample is higher than in a control sample.

19. The method of claim 17, wherein the subject has NORSE or is likely to develop NORSE when the level of at least one of IL6, CCL2, GCSF, and IL1b in the sample is higher than in the control sample.

20. (canceled)

21. The method of claim 8, further comprising administering one or more treatments to the subject.

22. The method of claim 21, wherein the treatment comprises administering an effective amount of a compound inhibiting upregulation of IL-1β, IL-6, and IL-10 and/or administering an effective amount of anakinra, tocilizumab, or biologics targeting cytokine upregulation in SE and NORSE to the subject.

23. (canceled)

24. The method of claim 21, wherein the treatment comprises administering an effective amount of a compound, composition, probiotic, and/or a prebiotic that stimulates growth and/or activity of one or more strains of fungi and/or bacteria which level determined in step (a) is lower than in the control.

25. The method of claim 24, wherein the treatment comprises administering an effective amount of a probiotic comprising one or more strains of fungi and/or bacteria, diet modification, or fecal microbiota transplantation (FMT), which level determined in step (a) is lower than in the control.

26. The method of claim 25, wherein the route of administration for said probiotic or FMT comprises at least one of upper gastrointestinal routes (UGI) (such as nasogastric/nasojejunal tube, endoscopy, or oral capsules) or lower gastrointestinal routes (LGI) (such as retention enema, sigmoidoscopy or colonoscopy).

27. The method of claim 24, wherein the level as determined in step (a) is at least 2-fold to 10-fold lower than in the control.

28. The method of claim 21, wherein the treatment comprises administering an effective amount of a compound or composition which inhibits growth and/or activity of one or more strains of fungi and/or bacteria which level determined in step (a) is higher than in the control.

29. The method of claim 28, wherein the level as determined in step (a) is at least 2-fold to 10-fold higher than in the control.

30.-79. (canceled)

80. The method of claim 8, wherein the subject is human.
